SEQUENCE LISTING (295 sequences)

SEQ ID NO: 1 - DNA, 397 bp, Zea mays
  source 1..397: mol_type = genomic DNA
aggaaaagaagaggtgattactgtacctattcgtctttgtatcggaatataaatttatcactattttatgataaagtaaatctgtttccctgtagagttaattaattaatgtaagtataagcgtaatttatagggcactagtaggactgtcgactgtgcgctcggcccggataatgcgtcaaaagcgaagacgtgcacgtgggatgggaaaacacgaagcgtggtctgctttttcgcatgatatctgggccgcaccaaagaatccagcccacgcggcgtggcgccgtcgttacggcttgcgggggaaggaaacgagggacgaaccgagatttagcaccagaccggccagcgagcattgcagacaccggcttataagttcagctgcgactaccactcc

SEQ ID NO: 2 - DNA, 200 bp, Zea mays
  source 1..200: mol_type = genomic DNA
cgtgggatgggaaaacacgaagcgtggtctgctttttcgcatgatatctgggccgcaccaaagaatccagcccacgcggcgtggcgccgtcgttacggcttgcgggggaaggaaacgagggacgaaccgagatttagcaccagaccggccagcgagcattgcagacaccggcttataagttcagctgcgactaccactcc

SEQ ID NO: 3 - DNA, 397 bp, Zea mays
  source 1..397: mol_type = genomic DNA
ttatatgttaccgttgcaaagcacgggcactcacctagtatataatataacatcagtcgtacgtaatgtactgatgggcgggttaacaaatgtcactcactatcagcaccagcagcgcttagatgcatccggccgggccaagacccaggaccagaaagcgcgcacgttcacagcggatgctgatgggttagatcgactgatcgaggaagaggagagcttaattaagaaacgccctgttccgctttgctagcttgcgccctgactgtccagcccacgcgcttcggtccgattcacatgctaggctggtgcaagcgagccgagacttttttttagaaccaccttgctcagcaaaccttaggaacaccggcttataagtcgaagcgaagcgctgtgcact

SEQ ID NO: 4 - DNA, 200 bp, Zea mays
  source 1..200: mol_type = genomic DNA
tgatcgaggaagaggagagcttaattaagaaacgccctgttccgctttgctagcttgcgccctgactgtccagcccacgcgcttcggtccgattcacatgctaggctggtgcaagcgagccgagacttttttttagaaccaccttgctcagcaaaccttaggaacaccggcttataagtcgaagcgaagcgctgtgcact

SEQ ID NO: 5 - DNA, 397 bp, Zea mays
  source 1..397: mol_type = genomic DNA
ggattcggtgttctttattaggttctcgccgaatatggttctcagttatgacctaacggtgtccacaagagttcgccaggatttatacaactattttcttatttatttctttaacattttcccttctacgcacaataggagataatgtcaagcgttgacggtgcacatatatttgtttttttaaaggcgtagtggcgtgtgtgcaaaaacatcctcacaggaaagacacgaagaaacatggtcaatggcccattatataaagcaccgccacaaagcccaaataccagttcgtcggtggagcaagtaacgcgctaggcaacaggcaaacagtttgtcccacctcgtccagtcacaaaggcaaagcgtgacttataagccagagcggaagaaccatacc

SEQ ID NO: 6 - DNA, 200 bp, Zea mays
  source 1..200: mol_type = genomic DNA
tgtgtgcaaaaacatcctcacaggaaagacacgaagaaacatggtcaatggcccattatataaagcaccgccacaaagcccaaataccagttcgtcggtggagcaagtaacgcgctaggcaacaggcaaacagtttgtcccacctcgtccagtcacaaaggcaaagcgtgacttataagccagagcggaagaaccatacc

SEQ ID NO: 7 - DNA, 397 bp, Zea mays
  source 1..397: mol_type = genomic DNA
tcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaatt

SEQ ID NO: 8 - DNA, 200 bp, Zea mays
  source 1..200: mol_type = genomic DNA
cggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaatt

SEQ ID NO: 9 - DNA, 540 bp, Solanum lycopersicum
  source 1..540: mol_type = genomic DNA
gttacgtaattatatttcttagtcatattttagttattccatcttaaccacataggtgatagtcaatatgtctatttcacatgtatggttccgtactataattaacaacatattgatttgaaattctatttgtgctacatatattagacaaggaaaataacatatgttattttgaaatcacgtatatttactataaattacaatgattaacaacttaaaatatttaaatgaaaatcatattaatgactctctaaattttatctgtgtcacataaatgaaaaacaaaaaataacaaatattgtattcgcacgggcgcatgtgtctagttagttataaacgaagaaataaggggctgatttcgaaataaacgttcttagaattggaagaaatgttcagtttctaaacttgtaggactaaagcaataacttttatttaatttattttcttttatgtttctcccacatcgatcatacatataactatacagcagtataagaactctagcgaagcaataatgctcgtcccgttggggacatccga

SEQ ID NO: 10 - DNA, 600 bp, Solanum lycopersicum
  source 1..600: mol_type = genomic DNA
tataataatactatgttaaatatgcaacatgtattagaagtgaattaagtatgcaatagatatgtatttaaaaatatattatgcttgtttgataagaagttgatgcattgtattataagtacgttagaatgtgcaataaatatattatctatcattagaacttgaattataagtgaataatagattattttttgtaatatgaattaaaagtgtattaaacatgtattaacggtgatcaattggttaaaaaaaagtttattattaaaatgataaatctttttaatttatagtatatttatgtaagttttcacgttgagtaaatagcgaagaagttgggcccaaccaagtaaaataagaaggccgggccattacaattaagtcgtcacacaactgggcttcattgaaaaaagcgcaaaaccgattccaggcccgtgttagcatgaagactcaactcaaccagagatttctccctcatcgcttacagaaaaaagctatatgctgtttatattgcgaaatctaacagtgtagtttgtcccttcggggacatccgataaaattggaacgatacagagaagattagcatggcccctgcgcaaggat

SEQ ID NO: 11 - DNA, 540 bp, Solanum lycopersicum
  source 1..540: mol_type = genomic DNA
ttctccaaaaaaaaaaaagaaaagaagaagcaaacaaacaaatagaagcatatctcttgatgtggaaggagatcaaaatattcccaataaatacttatgagaagaagtaactgatttaaaattttcactaatagggttcgaaaaatgaaaatgtaatacgtggaacttgaatgtaaaacctcaaggaattcttgtgtttaagaaattcaaaatctctctaaatgtatacaaaagatgatttctttttaccttatatatagtaaaataaaattgtcggataaattcgagtgaacaccctagcaccccctaaatcctcccccgtagtcggcccattacagttaaagtccaggtacaacaaaatgggcttcgattaagatggaataaaaggagtccaggcccatgagcccaacaaacaagctatttctccctcatcggcgcacaaagaagctttattctcttattatagctgaatattagcatgtgtgtttgtcccttcggggacatccgataaaattggaacgatacagagaagattagcat

SEQ ID NO: 12 - DNA, 540 bp, Glycine max
  source 1..540: mol_type = genomic DNA
acctttttaaccgcacaaagttttaaccagatttatataatttatttttgaatccccaatacatatcattataacatatcaattatcaaatatttcaataacctcatgatatggcaatgaatacatcttcttctcaatgaacagagatttctgaaaaagattaggaaagtgaaagcatactcgtttgcaatgtaaaactgatacttccccaaaatcatcatattccaaatatgccctggtgttactgaccaaaaccagaaaaaagaaacggaagacatatacgtctaaacggagaaatttcaaaaaacaaaaattggatcatttctcgatttgtgggtgtcatcttgtgcagggcatgctaatcttctctttaccctttcccacaagactcagcgcatgttgtctcgtctcatccaagtcccacaccgcctaaacttaacacaatattagtatttataatgacatacaacattcaagatgttgtcccttcggggacatccgataaaattggaacgatacagagaagattagcatggcccc

SEQ ID NO: 13 - DNA, 540 bp, Glycine max
  source 1..540: mol_type = genomic DNA
cattataaaaagtaaaatataactactttgttttttaataaaaaaaattcaatgggagatactatggattcaattaccttactgattttatttcatatgtgccagaagtatttcagtttattttgaaaaatcagaaaaaaaatgtctggaataaaatataataagcgatactaataaataattgaacaagataaatggtaaaatgtcaaatcaaaactaggctacagagtgcagagcagagtcatgatgaatgacagctagttctacttactacaccgattcttgtgtacataaaaatattttaaaataattgaatctttctttagccagctttgacaacaatgtacaccgttcgtacttcttactggtaggcaatgcttcttgtttgctttcggtggaaggtgtatatactcaacattacttctttttcagcgtgttttcttacgggagtcccacaccgcccaaaactaatacagtattcttgtttataaagaagtgcaccacttcaattgttgtcccttcggggacatccgataaaat

SEQ ID NO: 14 - DNA, 540 bp, Glycine max
  source 1..540: mol_type = genomic DNA
atattcataattttttttttttgtttttttatacaaggacggctgattcaatcatcacaccacacgtcatattaaaaaaatatagtagatttattttaaaatagagagaatcgttaagaaaaaaataaatagtaaagtaaatgaaaacccaaataatatcattattatgtcaataagtcggagaggatagtaatcaaatggtctatgaggtggtggttcattcaacatatagcacctattcattgttcctaaaacataatttaagaacaaaaacttaaacttaaataataataataaaagagtacatcgaagtatctgtgttctctatccttctgactaacattcatgttgtttgtattcagcaaagggccgtgcaggatttgtgcgtcgcgctccggttagttattgcagtgaccgtctctttagtcccacatcgagtaattatgcttcatacagtctgtttatataacagagatggaacaaactggttgtcccttcggggacatccgataaaattggaacgatacagagaagattagc

SEQ ID NO: 15 - DNA, 540 bp, Glycine max
  source 1..540: mol_type = genomic DNA
ctttcaggtcatgattttttgtttctaaatgatactcacactcccttccagttttttttttttaaactcagctcccttgcttcctccaccggttatcataatactgaaccaaatcaaacattacagtcaaggtactatgaatatgaaacctgaaatcctatgaatgtcataaatttattttaaataataaatttatttagaataatatttttttgggtaagagttataaaataaaatacaaaaaaaaaacctaatatcaatttttcactgactccgtttatattgagacttgagaaagatggttcccgtttgctcccggtggaggctccgaggctgtgtatatactcgacattactttagcttgttttgttgtttctttccctttcccacaagactcaggtctcgttcgcaaacgagtcccacaccgtctaaacttaccacaatattagcgtttataattagatgcactgcatcacttattgtcccttcggggacatccgataaaattggaacgatacagagaagattagcatggcccct

SEQ ID NO: 16 - DNA, 420 bp, Glycine max
  source 1..420: mol_type = genomic DNA
tgcagagcagagtcatgatgatactacttactacaccgattcttgtgtgcagaaaaatatgttaaaataattgaatctttctctagccaaatttgacaacaatgtacaccgttcatattgagagacgatgcttcttgtttgctttcggtggaagctgcatatactcaacattactccttcagcgagttttccaactgagtcccacattgcccagacctaacacggtattcttgtttataatgaaatgtgccaccacatggattgtcccttcggggacatccgataaaattggaacgatacagagaagattagcatggcccctgcgcaaggatgacacgcacaaatcgagaaatggtccaaattttttttgaaatttctcgtttagatagatgtctttgcttttccgcactatggttctga

SEQ ID NO: 17 - DNA, 397 bp, synthetic construct
  source 1..397: mol_type = other DNA; note = Recombinant
aggaaaagaagaggtgattactgtacctattcgtctttgtatcggaatataaatttatcactattttatgataaagtaaatctgtttccctgtagagttaattaattaatgtaagtataagcgtaatttatagggcactagtaggactgtcgactgtgcgctcggcccggataatgcgtcaaaagcgaagacgtgcacgtgggatgggaaaacacgaagcgtggtctgctttttcgcatgatatctgggccgcaccaaagaatccagcccacgcggcgtggcgccgtcgttacggcttgcgggggaaggaaacgagggacgaaccgagatttagtcccaccttgactaatcacaagagtggagcgtaccttataaaccgagccgcaagcaccgaatt

SEQ ID NO: 18 - DNA, 397 bp, synthetic construct
  source 1..397: mol_type = other DNA; note = Recombinant
aggaaaagaagaggtgattactgtacctattcgtctttgtatcggaatataaatttatcactattttatgataaagtaaatctgtttccctgtagagttaattaattaatgtaagtataagcgtaatttatagggcactagtaggactgtcgactgtgcgctcggcccggataatgcgtcaaaagcgaagacgtgcacgtgggatgggaaaacacgaagcgtggtctgctttttcgcatgatatctgggccgcaccaaagaatccagcccacgcggaagcccaaacagcagtccgtaggtggagcaaagcgctgggtaatacgcaaacgttttgtcccaccttgactaatcacaagagtggagcgtaccttataaaccgagccgcaagcaccgaatt

SEQ ID NO: 19 - DNA, 397 bp, synthetic construct
  source 1..397: mol_type = other DNA; note = Recombinant
aagtcgtaaaatagtggtgtccaaagaatttccaggcccagttgtaaaagctaaaatgctattcgaatttctactagcagtaagtcgtgtttagaaattattttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgcaccagaccggccagcgagcattgcagacaccggcttataagttcagctgcgactaccactcc

SEQ ID NO: 20 - DNA, 397 bp, synthetic construct
  source 1..397: mol_type = other DNA; note = Recombinant
tcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaactgtcacagagagggccataagaaacatggcccacggcccaataagcccaccagcccaccgaagcccaaacagcagtccgtaggtggagcaaagcgctgggtaatacgcaaacgttttgtcccaccttgactaatcacaagagtggagcgtaccttataaaccgagccgcaagcaccgaatt

SEQ ID NO: 21 - DNA, 83 bp, synthetic construct
  source 1..83: mol_type = other DNA; note = Recombinant
gttttagagctagaaatagcaagttaaaataaggctagtccgttatcaacttgaaaaagtggcaccgagtcggtgcttttttt

SEQ ID NO: 22 - DNA, 23 bp, Zea mays
  source 1..23: mol_type = genomic DNA
gccggccagcatttgaaacatgg

SEQ ID NO: 23 - DNA, 20 bp, Zea mays
  source 1..20: mol_type = genomic DNA
gccggccagcatttgaaaca

SEQ ID NO: 24 - DNA, 20 bp, Zea mays
  source 1..20: mol_type = unassigned DNA
gttatcaatttactttcaat

SEQ ID NO: 25 - DNA, 20 bp, Zea mays
  source 1..20: mol_type = unassigned DNA
gcgcaagggatcagtaattc

SEQ ID NO: 26 - protein, 1064 aa, Bradyrhizobium sp.
MKRTSLRAYRLGVDLGANSLGWFVVWLDDHGQPEGLGPGGVRIFPDGRNPQSKQSNAAGRRLARSARRRRDRYLQRRGKLMGLLVKHGLMPADEPARKRLECLDPYGLRAKALDEVLPLHHVGRALFHLNQRRGLFANRAIEQGDKDASAIKAAAGRLQTSMQACGARTLGEFLNRRHQLRATVRARSPVGGDVQARYEFYPTRAMVDAEFEAIWAAQAPHHPTMTAEAHDTIREAIFSQRAMKRPSIGKCSLDPATSQDDVDGFRCAWSHPLAQRFRIWQDVRNLAVVETGPTSSRLGKEDQDKVARALLQTDQLSFDEIRGLLGLPSDARFNLESDRRDHLKGDATGAILSARRHFGPAWHDRSLDRQIDIVALLESALDEAAIIASLGTTHSLDEAAAQRALSALLPDGYCRLGLRAIKRVLPLMEAGRTYAEAASAAGYDHALLPGGKLSPTGYLPYYGQWLQNDVVGSDDERDTNERRWGRLPNPTVHIGIGQLRRVVNELIRWHGPPAEITVELTRDLKLSPRRLAELEREQAENQRKNDKRTSLLRKLGLPASTHNLLKLRLWDEQGDVASECPYTGEAIGLERLVSDDVDIDHLIPFSISWDDSAANKVVCMRYANREKGNRTPFEAFGHRQGRPYDWADIAERAARLPRGKRWRFGPGARAQFEELGDFQARLLNETSWLARVAKQYLAAVTHPHRIHVLPGRLTALLRATWELNDLLPGSDDRAAKSRKDHRHHAIDALVAALTDQALLRRMANAHDDTRRKIEVLLPWPTFRIDLETRLKAMLVSHKPDHGLQARLHEDTAYGTVEHPETEDGANLVYRKTFVDISEKEIDRIRDRRLRDLVRAHVAGERQQGKTLKAAVLSFAQRRDIAGHPNGIRHVRLTKSIKPDYLVPIRDKAGRIYKSYNAGENAFVDILQAESGRWIARATTVFQANQANESHDAPAAQPIMRVFKGDMLRIDHAGAEKFVKIVRLSPSNNLLYLVEHHQAGVFQTRHDDPEDSFRWLFASFDKLREWNAELVRIDTLGQPWRRKRGLETGSEDATRIGWTRPKKWP

SEQ ID NO: 27 - DNA, 3195 bp, synthetic construct
  source 1..3195: mol_type = other DNA; note = Recombinant
atgaagagaacgagtttacgggcctaccgtctgggcgtggatctcggcgccaattcgctgggatggttcgtggtctggctcgacgatcacggacagcccgagggccttggcccgggcggcgtcaggattttccccgacggtcgtaacccgcaatccaagcaatccaatgcggccggtcgccgcctcgcacgcagtgcacgacgacgacgagaccgctatctgcagcgacgcggaaagctgatgggcttgctggtcaagcacggcttgatgcccgccgatgagccggcccgaaagcgattggaatgcctcgatccctatggtctccgcgcgaaagcgctcgatgaagtgctgcctttgcatcatgtcggccgggcgctgtttcacctcaaccagcggcgcggcctgtttgccaatcgagcgatcgagcaaggcgacaaggacgccagcgcgatcaaggccgcggccggcagactgcagacatcgatgcaggcgtgcggcgcgcgcacgctcggcgaattcctcaaccgccgtcatcagctccgcgccacagtgcgcgcccgcagccctgtcggcggcgacgtccaggcgcggtatgaattctatccgacacgcgcgatggttgatgcggagttcgaagccatctgggcggcacaggcaccgcatcacccaacgatgacggccgaagcgcatgacacgatccgcgaggcgatcttctctcaacgcgcgatgaagcggccgtcgatcgggaaatgctcgctcgaccccgccaccagccaggacgacgtcgacggctttcgctgcgcctggtcgcatcccctggcgcagcgtttccgcatctggcaggacgtccgcaatctagccgtggtggagactggccccacgtcttccaggcttggcaaggaggatcaggacaaggtcgcacgggcactgctacagaccgaccaactcagcttcgatgagatccgcggccttctcggattgccgtcggacgcgcggttcaaccttgaaagcgaccggcgtgatcacctcaagggcgacgcgaccggcgcgatcctgtccgccaggaggcattttggcccggcatggcatgaccggtccctggatcgtcagatcgacatcgtcgcgctgctggagagcgcgctcgatgaagcagcgatcatcgcctcgctcgggacaactcacagccttgatgaagcagctgcgcagcgggcgttgtccgccttgctgcctgacggatattgcaggcttggactgagggcgatcaagcgggtcctgccgctcatggaagctggcaggacctacgcggaggccgccagcgcggccggctatgatcacgctctgctgccgggcggcaagctctctcccaccggctacctgccctattatggacaatggctgcagaacgatgtcgtgggctcggacgatgagcgcgacaccaacgaacggcgctggggccgcttgccgaatcccaccgttcacatcgggatcggccagttgcgacgcgtcgtcaatgagctcatcagatggcatggaccgccggccgagatcaccgtcgagttgacgcgtgacctgaagctgtcgccccgacggctggcggagctcgaacgcgagcaggccgagaaccagcgcaagaacgacaagcgtacctccctattgcgcaagctcgggctccccgcgagcacgcacaatctcctcaagcttcggctctgggacgagcaaggcgatgttgcaagcgaatgcccctatacgggcgaggcgatcggcctcgaacgtctggtctctgatgatgtggatatcgatcacctcatcccattctcgatcagctgggacgacagcgcggccaacaaagtggtctgcatgcgctacgccaatcgtgagaagggcaatcgaacgccgttcgaggcctttggccatcgccaaggcaggccttacgattgggcggacattgcagaacgcgcagcgcgcctgccgcgcggcaagcgctggcgcttcggtccaggcgcgcgggcgcaattcgaggagctcggcgactttcaggcacgcctgctcaacgagaccagctggctggcgcgcgtcgccaagcaatatctcgcagcggtcacccacccgcacaggatccacgttctgccgggccggctgacagcgctgctccgcgcaacatgggagctcaacgatttgctgcccggaagcgacgacagagccgcgaagagccgcaaggaccaccgtcatcatgccatcgacgcgctggtggcggcactgacagaccaggcgctgctgcgccgcatggcgaacgcgcatgacgatacgcgacggaagatcgaagttctcctgccctggccgacgttccggatcgatctcgagaccaggctgaaggcgatgctcgtatcgcacaagcccgatcacggcctccaggcccgcctgcatgaagacaccgcctatgggaccgtcgaacaccccgaaaccgaggatggtgcaaatctggtctatcggaagaccttcgtggacatcagcgaaaaggagatcgaccgcattcgcgatcgccgcttgcgtgacctcgtcagagcccatgtggccggcgaaaggcagcagggcaagacgctcaaagcggcggtgctgtcattcgcgcagcgcagggacattgctggtcacccgaatggcattcgccatgtccgcctgaccaaatcgatcaagccggactatctggtaccgatccgcgacaaagccggccgcatctacaagtcctacaatgcaggcgagaatgccttcgtcgacatcctgcaagccgagagtggccgatggatcgcgcgggccacgaccgtctttcaggccaatcaagccaatgagtcgcatgacgcgccagcggcgcaaccgatcatgcgggtcttcaagggcgacatgctgcgcatcgatcacgctggcgcggagaagttcgtgaagatcgtcaggctttcgccctcgaacaacctgctctacctcgtcgaacatcatcaggcgggcgtgtttcagacccgccatgacgacccggaagattcctttcggtggctcttcgccagttttgacaagcttcgcgaatggaacgccgagcttgtccggatcgatacgctgggacagccctggcggcgcaagcgcggccttgaaacaggaagcgaggacgccactcgcatcggctggacgcgaccaaaaaaatggccctga

SEQ ID NO: 28 - protein, 1378 aa, synthetic construct
  source 1..1378: note = Recombinant
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSKKRRIKQD

SEQ ID NO: 29 - DNA, 4137 bp, synthetic construct
  source 1..4137: mol_type = other DNA; note = Recombinant
atggacaagaagtacagcattggtctggacatcggaacgaactcggtcggctgggccgtgattactgacgagtacaaagttcccagcaaaaaattcaaggttctaggcaacacagaccgccactcgattaaaaagaatctaatcggcgcgcttctgttcgactctggtgaaacggccgaggccacacgcttaaagaggaccgcgcgccgccgctacacgcggcgcaagaaccgaatctgttacctccaggagatcttcagtaatgagatggctaaggtcgatgacagcttcttccacaggcttgaagagtcctttctggtcgaagaggacaaaaaacacgaacgtcacccaatcttcgggaacattgtggatgaagtcgcataccacgagaagtatcctacgatctatcacctccgcaagaagctcgtggatagtaccgacaaagccgacctgcgcttaatctaccttgcgctcgcgcacatgattaagttccgaggacacttccttattgagggtgatctgaatccggacaattccgatgtggataaactgttcattcagttggtccagacatacaatcagctattcgaggagaatccgatcaatgcttccggcgtggacgcaaaggctattctgtcagcaagactttcaaagagcagaaggttggagaatctgatcgcacaacttcccggagagaagaagaatgggctcttcggcaacctcattgcgctgtctttgggtctgacaccgaactttaagtctaacttcgacctcgctgaggatgctaaacttcagcttagcaaagacacctatgatgatgacctggacaacctcctcgcccagattggagaccagtacgcggatctattcttggctgccaagaacctgtccgatgcgattctgcttagtgacatcctccgagtgaacactgaaattacgaaagcacccttgtcggctagtatgattaagcgatacgatgagcaccatcaagacctgacattgctaaaggcgctcgtaagacagcaacttcctgagaagtacaaggagatattttttgatcagtctaagaatggctacgctggttacatcgacggtggagctagtcaggaggaattctataaattcatcaagcctatcctggaaaaaatggacggtacggaggaattgctcgttaaactaaatcgagaggatctgctgagaaagcagcggactttcgacaatggttctattccgcatcagattcacctcggagaacttcacgccatcctgagacgacaggaggacttctaccctttcctgaaagacaaccgggaaaaaatcgagaagatcctgacattcaggattccttactatgtaggccctttagcgagaggcaacagtagattcgcctggatgaccagaaagtctgaggaaacaatcacaccgtggaacttcgaggaagtggttgataagggtgctagtgcccaatcattcattgagagaatgacgaacttcgacaagaatctgcctaacgagaaggttctccctaaacatagcttgctttacgagtatttcacggtgtacaatgagctaacgaaggtcaagtatgtcacagagggaatgcggaaaccggctttcctttcgggtgaacagaagaaagcaattgtggatttgctcttcaagacaaaccgaaaggtgacagtgaagcagctaaaggaggactacttcaaaaaaatagagtgcttcgactcagttgagatcagcggagtggaggaccggtttaacgcttccctcggcacttaccacgacttgctcaagatcatcaaggacaaagacttccttgataacgaggagaacgaagacatccttgaggacattgtgctgacattgacgttgttcgaggatcgggagatgatcgaggaacgcctcaagacgtacgcccatctgttcgatgataaggtgatgaagcagttaaagaggagacgttacactggctggggccgtctctctcgcaaactgataaacgggataagggataaacaaagcggaaagacaatcctcgatttccttaaatccgacggcttcgctaaccggaacttcatgcagctcatccatgatgactcactgacgttcaaggaggacatccagaaagctcaagtgtctggccagggtgacagcttgcacgagcacatcgcaaatctagccggttcaccggcgataaagaagggcattctacaaacggtgaaagtggtggacgagcttgtgaaggtcatgggtcgccataagccagagaacattgttatcgaaatggcgagggagaaccagacaacgcagaagggacaaaaaaacagtagggagcggatgaagcgcatcgaggaaggcattaaggaacttggaagccaaatcctgaaagagcacccggtggagaatacgcagttgcagaacgagaaactgtacctctactacttgcagaatggacgtgatatgtatgtggatcaagagttggacatcaaccgattgtctgactatgacgtggatcacatagtaccacagtccttcctcaaggatgatagcatagacaacaaggttcttactcgtagcgacaagaatcgtggcaaatcggacaatgttccatctgaggaagttgtcaaaaagatgaaaaattattggaggcaacttctgaacgcgaagctaattacacaaaggaaattcgacaatctcactaaggccgagagaggagggttaagtgagttagacaaggctggcttcatcaagcggcagttggtcgagactcgtcagattactaagcacgtggctcagatcctggattcgcgcatgaacaccaaatacgacgagaatgacaaactcatccgtgaagttaaggtgattacactgaaatccaagctggtctctgactttaggaaagacttccaattctacaaggtgagagagattaacaactaccaccacgcgcatgacgcctacttgaatgctgtggttgggactgccctgataaagaaatatcctaaacttgagtctgagttcgtttacggtgactacaaggtttatgacgttaggaagatgatcgccaaatccgaacaggagattgggaaagcaactgccaaatatttcttttactccaacattatgaactttttcaagacagaaatcacactcgccaatggcgagattcggaagagaccactaatcgaaacaaacggcgaaactggtgaaatcgtttgggataagggtagggacttcgcaactgttaggaaggtcttgtcgatgcctcaagtgaacatagtcaaaaaaacagaggtccagaccggtgggttctcaaaggagtctattctgccaaagcgtaacagcgacaaactcatcgctcgcaaaaaggactgggatcctaaaaagtacggtggattcgacagcccgaccgttgcttattctgttctcgtagttgctaaagtcgagaagggcaagtccaaaaaactcaaatcggttaaggaactgctcggaatcacgataatggaacgaagcagtttcgaaaaaaatccaattgacttcctggaagctaaaggttacaaggaggtcaagaaggatcttatcatcaagctacccaagtacagtctgttcgaactggagaacggtcgcaagagaatgctggcctcggctggtgaactccagaagggcaatgagctggccctgccgtccaagtacgtgaactttctgtacctggcatctcattacgagaagctcaagggctcaccagaggacaacgagcagaagcagttgttcgttgaacagcacaaacactatcttgatgagatcattgagcaaattagcgagttcagtaagcgagttattctggctgatgctaacctggataaggtgctctctgcctacaacaagcaccgggataaacctattagggaacaggcggagaacataatccacctcttcactctgacgaatctcggcgcgcctgcggccttcaaatattttgataccaccatcgacaggaagcgctacacaagcactaaagaggtgctggacgccactctcattcaccagtctattaccgggctctacgagacacggattgacctctcccagctaggtggcgatggatctaagaagagaagaattaaacaagattaa

SEQ ID NO: 30 - DNA, 500 bp, synthetic construct
  source 1..500: mol_type = other DNA; note = Recombinant
aggaaaagaagaggtgattactgtacctattcgtctttgtatcggaatataaatttatcactattttatgataaagtaaatctgtttccctgtagagttaattaattaatgtaagtataagcgtaatttatagggcactagtaggactgtcgactgtgcgctcggcccggataatgcgtcaaaagcgaagacgtgcacgtgggatgggaaaacacgaagcgtggtctgctttttcgcatgatatctgggccgcaccaaagaatccagcccacgcggcgtggcgccgtcgttacggcttgcgggggaaggaaacgagggacgaaccgagatttagcaccagaccggccagcgagcattgcagacaccggcttataagttcagctgcgactaccactccgccggccagcatttgaaacagttttagagctagaaatagcaagttaaaataaggctagtccgttatcaacttgaaaaagtggcaccgagtcggtgcttttttt

SEQ ID NO: 31 - DNA, 303 bp, synthetic construct
  source 1..303: mol_type = other DNA; note = Recombinant
cgtgggatgggaaaacacgaagcgtggtctgctttttcgcatgatatctgggccgcaccaaagaatccagcccacgcggcgtggcgccgtcgttacggcttgcgggggaaggaaacgagggacgaaccgagatttagcaccagaccggccagcgagcattgcagacaccggcttataagttcagctgcgactaccactccgccggccagcatttgaaacagttttagagctagaaatagcaagttaaaataaggctagtccgttatcaacttgaaaaagtggcaccgagtcggtgcttttttt

SEQ ID NO: 32 - DNA, 500 bp, synthetic construct
  source 1..500: mol_type = other DNA; note = Recombinant
ttatatgttaccgttgcaaagcacgggcactcacctagtatataatataacatcagtcgtacgtaatgtactgatgggcgggttaacaaatgtcactcactatcagcaccagcagcgcttagatgcatccggccgggccaagacccaggaccagaaagcgcgcacgttcacagcggatgctgatgggttagatcgactgatcgaggaagaggagagcttaattaagaaacgccctgttccgctttgctagcttgcgccctgactgtccagcccacgcgcttcggtccgattcacatgctaggctggtgcaagcgagccgagacttttttttagaaccaccttgctcagcaaaccttaggaacaccggcttataagtcgaagcgaagcgctgtgcactgccggccagcatttgaaacagttttagagctagaaatagcaagttaaaataaggctagtccgttatcaacttgaaaaagtggcaccgagtcggtgcttttttt

SEQ ID NO: 33 - DNA, 303 bp, synthetic construct
  source 1..303: mol_type = other DNA; note = Recombinant
tgatcgaggaagaggagagcttaattaagaaacgccctgttccgctttgctagcttgcgccctgactgtccagcccacgcgcttcggtccgattcacatgctaggctggtgcaagcgagccgagacttttttttagaaccaccttgctcagcaaaccttaggaacaccggcttataagtcgaagcgaagcgctgtgcactgccggccagcatttgaaacagttttagagctagaaatagcaagttaaaataaggctagtccgttatcaacttgaaaaagtggcaccgagtcggtgcttttttt

SEQ ID NO: 34 - DNA, 500 bp, synthetic construct
  source 1..500: mol_type = other DNA; note = Recombinant
ggattcggtgttctttattaggttctcgccgaatatggttctcagttatgacctaacggtgtccacaagagttcgccaggatttatacaactattttcttatttatttctttaacattttcccttctacgcacaataggagataatgtcaagcgttgacggtgcacatatatttgtttttttaaaggcgtagtggcgtgtgtgcaaaaacatcctcacaggaaagacacgaagaaacatggtcaatggcccattatataaagcaccgccacaaagcccaaataccagttcgtcggtggagcaagtaacgcgctaggcaacaggcaaacagtttgtcccacctcgtccagtcacaaaggcaaagcgtgacttataagccagagcggaagaaccataccgccggccagcatttgaaacagttttagagctagaaatagcaagttaaaataaggctagtccgttatcaacttgaaaaagtggcaccgagtcggtgcttttttt

SEQ ID NO: 35 - DNA, 303 bp, synthetic construct
  source 1..303: mol_type = other DNA; note = Recombinant
tgtgtgcaaaaacatcctcacaggaaagacacgaagaaacatggtcaatggcccattatataaagcaccgccacaaagcccaaataccagttcgtcggtggagcaagtaacgcgctaggcaacaggcaaacagtttgtcccacctcgtccagtcacaaaggcaaagcgtgacttataagccagagcggaagaaccataccgccggccagcatttgaaacagttttagagctagaaatagcaagttaaaataaggctagtccgttatcaacttgaaaaagtggcaccgagtcggtgcttttttt

SEQ ID NO: 36 - DNA, 500 bp, synthetic construct
  source 1..500: mol_type = other DNA; note = Recombinant
tcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgccggccagcatttgaaacagttttagagctagaaatagcaagttaaaataaggctagtccgttatcaacttgaaaaagtggcaccgagtcggtgcttttttt

SEQ ID NO: 37 - DNA, 303 bp, synthetic construct
  source 1..303: mol_type = other DNA; note = Recombinant
cggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgccggccagcatttgaaacagttttagagctagaaatagcaagttaaaataaggctagtccgttatcaacttgaaaaagtggcaccgagtcggtgcttttttt

SEQ ID NO: 38 - DNA, 500 bp, synthetic construct
  source 1..500: mol_type = other DNA; note = Recombinant
aggaaaagaagaggtgattactgtacctattcgtctttgtatcggaatataaatttatcactattttatgataaagtaaatctgtttccctgtagagttaattaattaatgtaagtataagcgtaatttatagggcactagtaggactgtcgactgtgcgctcggcccggataatgcgtcaaaagcgaagacgtgcacgtgggatgggaaaacacgaagcgtggtctgctttttcgcatgatatctgggccgcaccaaagaatccagcccacgcggcgtggcgccgtcgttacggcttgcgggggaaggaaacgagggacgaaccgagatttagcaccagaccggccagcgagcattgcagacaccggcttataagttcagctgcgactaccactccgcgcaagggatcagtaattcgttttagagctagaaatagcaagttaaaataaggctagtccgttatcaacttgaaaaagtggcaccgagtcggtgcttttttt

SEQ ID NO: 39 - DNA, 500 bp, synthetic construct
  source 1..500: mol_type = other DNA; note = Recombinant
ttatatgttaccgttgcaaagcacgggcactcacctagtatataatataacatcagtcgtacgtaatgtactgatgggcgggttaacaaatgtcactcactatcagcaccagcagcgcttagatgcatccggccgggccaagacccaggaccagaaagcgcgcacgttcacagcggatgctgatgggttagatcgactgatcgaggaagaggagagcttaattaagaaacgccctgttccgctttgctagcttgcgccctgactgtccagcccacgcgcttcggtccgattcacatgctaggctggtgcaagcgagccgagacttttttttagaaccaccttgctcagcaaaccttaggaacaccggcttataagtcgaagcgaagcgctgtgcactgcgcaagggatcagtaattcgttttagagctagaaatagcaagttaaaataaggctagtccgttatcaacttgaaaaagtggcaccgagtcggtgcttttttt

SEQ ID NO: 40 - DNA, 500 bp, synthetic construct
  source 1..500: mol_type = other DNA; note = Recombinant
ggattcggtgttctttattaggttctcgccgaatatggttctcagttatgacctaacggtgtccacaagagttcgccaggatttatacaactattttcttatttatttctttaacattttcccttctacgcacaataggagataatgtcaagcgttgacggtgcacatatatttgtttttttaaaggcgtagtggcgtgtgtgcaaaaacatcctcacaggaaagacacgaagaaacatggtcaatggcccattatataaagcaccgccacaaagcccaaataccagttcgtcggtggagcaagtaacgcgctaggcaacaggcaaacagtttgtcccacctcgtccagtcacaaaggcaaagcgtgacttataagccagagcggaagaaccataccgcgcaagggatcagtaattcgttttagagctagaaatagcaagttaaaataaggctagtccgttatcaacttgaaaaagtggcaccgagtcggtgcttttttt

SEQ ID NO: 41 - DNA, 500 bp, synthetic construct
  source 1..500: mol_type = other DNA; note = Recombinant
tcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgcgcaagggatcagtaattcgttttagagctagaaatagcaagttaaaataaggctagtccgttatcaacttgaaaaagtggcaccgagtcggtgcttttttt

SEQ ID NO: 42 - DNA, 500 bp, synthetic construct
  source 1..500: mol_type = other DNA; note = Recombinant
tcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgttatcaatttactttcaatgttttagagctagaaatagcaagttaaaataaggctagtccgttatcaacttgaaaaagtggcaccgagtcggtgcttttttt

SEQ ID NO: 43 - DNA, 23 bp, Zea mays
  source 1..23: mol_type = genomic DNA
gttatcaatttactttcaatcgg

SEQ ID NO: 44 - DNA, 23 bp, Zea mays
  source 1..23: mol_type = genomic DNA
gcgcaagggatcagtaattcagg

SEQ ID NO: 45 - DNA, 86 bp, synthetic construct
  source 1..86: mol_type = other DNA; note = Recombinant
gggataacagggtaatatagcgtaactataacggtcctaaggtagcgaattacgatacaaggctacctagcttcgcagttacgcta

SEQ ID NO: 46 - DNA, 86 bp, synthetic construct
  source 1..86: mol_type = other DNA; note = Recombinant
tagcgtaactgcgaagctaggtagccttgtatcgtaattcgctaccttaggaccgttatagttacgctatattaccctgttatccc

SEQ ID NO: 47 - DNA, 23 bp, Zea mays
  source 1..23: mol_type = genomic DNA
gatatgccaaacggtacttgagg

SEQ ID NO: 48 - DNA, 500 bp, synthetic construct
  source 1..500: mol_type = other DNA; note = Recombinant
tcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgtacaaaccaactcggcaatgttttagagctagaaatagcaagttaaaataaggctagtccgttatcaacttgaaaaagtggcaccgagtcggtgcttttttt

SEQ ID NO: 49 - DNA, 20 bp, synthetic construct
  source 1..20: mol_type = other DNA; note = Recombinant
gcgtaactgcgaagctaggt

SEQ ID NO: 50 - DNA, 20 bp, synthetic construct
  source 1..20: mol_type = other DNA; note = Recombinant
ctcacgccttcatttcaaag

SEQ ID NO: 51 - DNA, 20 bp, synthetic construct
  source 1..20: mol_type = other DNA; note = Recombinant
gtaatgggtaatcacaaagg

SEQ ID NO: 52 - DNA, 22 bp, synthetic construct
  source 1..22: mol_type = other DNA; note = Recombinant
ggattatattagtttaggcttg

SEQ ID NO: 53 - DNA, 20 bp, synthetic construct
  source 1..20: mol_type = other DNA; note = Recombinant
cgaccaactctccaccaatc

SEQ ID NO: 54 - DNA, 21 bp, synthetic construct
  source 1..21: mol_type = other DNA; note = Recombinant
taaggagatgagtttgagacc

SEQ ID NO: 55 - DNA, 20 bp, synthetic construct
  source 1..20: mol_type = other DNA; note = Recombinant
tagaggtggaagcatcaaag

SEQ ID NO: 56 - DNA, 20 bp, synthetic construct
  source 1..20: mol_type = other DNA; note = Recombinant
gtaccgtttggcatatcaac

SEQ ID NO: 57 - DNA, 20 bp, synthetic construct
  source 1..20: mol_type = other DNA; note = Recombinant
gagcatctaaccaccaaaac

SEQ ID NO: 58 - DNA, 500 bp, synthetic construct
  source 1..500: mol_type = other DNA; note = Recombinant
tcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgatatgccaaacggtacttggttttagagctagaaatagcaagttaaaataaggctagtccgttatcaacttgaaaaagtggcaccgagtcggtgcttttttt

SEQ ID NO: 59 - DNA, 23 bp, Zea mays
  source 1..23: mol_type = genomic DNA
gatatgccaaacggtacttgagg

SEQ ID NO: 60 - DNA, 500 bp, synthetic construct
  source 1..500: mol_type = other DNA; note = Recombinant
tcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattggatctcctatcataacgttgttttagagctagaaatagcaagttaaaataaggctagtccgttatcaacttgaaaaagtggcaccgagtcggtgcttttttt

SEQ ID NO: 61 - DNA, 23 bp, Zea mays
  source 1..23: mol_type = genomic DNA
ggatctcctatcataacgtttgg

SEQ ID NO: 62 - DNA, 92 bp, synthetic construct
  source 1..92: mol_type = other DNA; note = Recombinant
ggcgggataacagggtaatatagcgtaactataacggtcctaaggtagcgaattacgatacaaggctacctagcttcgcagttacgctaaat

SEQ ID NO: 63 - DNA, 92 bp, synthetic construct
  source 1..92: mol_type = other DNA; note = Recombinant
atttagcgtaactgcgaagctaggtagccttgtatcgtaattcgctaccttaggaccgttatagttacgctatattaccctgttatcccgcc

SEQ ID NO: 64 - DNA, 92 bp, synthetic construct
  source 1..92: mol_type = other DNA; note = Recombinant
tacgggataacagggtaatatagcgtaactataacggtcctaaggtagcgaattacgatacaaggctacctagcttcgcagttacgctattg

SEQ ID NO: 65 - DNA, 92 bp, synthetic construct
  source 1..92: mol_type = other DNA; note = Recombinant
caatagcgtaactgcgaagctaggtagccttgtatcgtaattcgctaccttaggaccgttatagttacgctatattaccctgttatcccgta

SEQ ID NO: 66 - DNA, 92 bp, synthetic construct
  source 1..92: mol_type = other DNA; note = Recombinant
aacgggataacagggtaatatagcgtaactataacggtcctaaggtagcgaattacgatacaaggctacctagcttcgcagttacgctagtt

SEQ ID NO: 67 - DNA, 92 bp, synthetic construct
  source 1..92: mol_type = other DNA; note = Recombinant
aactagcgtaactgcgaagctaggtagccttgtatcgtaattcgctaccttaggaccgttatagttacgctatattaccctgttatcccgtt

SEQ ID NO: 68 - DNA, 3366 bp, synthetic construct
  source 1..3366: mol_type = other DNA; note = Recombinant
atgagtgacctggtgctagggttggatataggcattggctccgtgggggttggcattcttaataaggtgaccggcgaaataattcataaaaactcacgcatctttccagcagcccaggctgagaacaatctggtccgtagaaccaaccggcagggtcgaaggttagccaggcgcaagaagcacagacgggtccggctcaacaggcttttcgaggagtctggtttgatcaccgatttcactaagatttctatcaacctgaatccttatcagctgcgcgttaaaggtctcacagacgaacttagcaacgaagagttgttcatcgccctgaaaaatatggtcaagcatcgcggcattagctacctggacgacgcttcggatgatggcaacagtagtgtaggtgactacgctcagatcgtgaaagagaactcgaagcaattggagaccaagaccccgggccaaattcaactcgaaaggtaccagacgtatggacagttacgaggcgattttaccgttgaaaaggatggtaagaagcacaggctgattaatgtgtttccgacctcagcttatcgctctgaggcgctgcgtattttgcagacccaacaggaatttaacccgcaaataacggacgagttcataaaccgatacttagagattcttacaggtaaacgtaaatactatcacggcccaggaaatgaaaagtccaggacagattatggtcgatatcgcacttccggagagactctcgacaatatctttggcattcttataggcaaatgtaccttctaccctgacgaatttagagcagcgaaggcttcatatacagcacaagagtttaatcttctcaacgacctcaacaacttgactgtgcctactgaaaccaaaaagcttagcaaggagcaaaaaaatcaaatcattaactatgttaagaatgagaaagctatggggcccgcaaaattgttcaagtacatagctaagttacttagctgtgacgttgctgatattaagggttaccgtattgacaagtctggtaaagctgaaattcacacctttgaggcttataggaagatgaagacccttgagacacttgacattgagcagatggatagggagactttggacaaactggcatacgtcttgacattgaacaccgaaagggaaggcatccaggaagctctggaacatgaatttgcagatggttcgttcagccaaaaacaggttgacgagctggtccaatttagaaaggcaaactcaagcatattcggtaaaggttggcacaacttcagcgttaagctgatgatggaactcattccagaattatatgaaacctctgaggaacagatgacgattctcacaagattgggtaagcagaaaacaaccagctctagcaataagactaaatacattgacgaaaagctcctcaccgaagagatttataacccggtcgtggcaaagagtgtacggcaagccatcaagatcgttaatgccgctatcaaggagtatggtgattttgataatattgtgattgaaatggcacgcgagactaacgaggacgacgagaagaaagctatacagaagattcaaaaggctaataaggacgagaaggacgccgcaatgctaaaggcggccaatcaatataatgggaaggctgaactacctcatagcgtcttccatggacataagcaattagcaactaaaataagattatggcaccagcaaggcgaacggtgtctttatacaggtaaaacgatatctattcacgacctgattaacaactctaaccagtttgaagtggatcatatcttaccactaagtatcaccttcgacgattcacttgctaacaaggtgctcgtttacgccactgcgaaccaagagaaagggcagaggactccataccaggcccttgacagcatggacgacgcctggagttttagggaattaaaagctttcgtacgtgagtcaaagacgctttcaaataaaaaaaaggagtacttgctcactgaagaagacatctcaaaattcgacgtgcgcaaaaaattcattgagcggaacttagtcgacactcggtacgcatcaagagtagtgttgaacgccctccaggagcactttagggcacataagatcgacaccaaggtttcagttgttaggggtcagtttacatcgcagcttagacgccattggggtatagaaaaaacacgtgatacctaccatcaccatgcagttgacgctctcatcattgcagcatcttctcaacttaatttgtggaaaaagcaaaagaacactctggtctcatatagcgaagatcagctgcttgatattgaaaccggcgagctgatttctgacgacgaatacaaagaatctgtgtttaaggcaccatatcaacactttgtagacacgcttaaatctaaagagtttgaggattcgatccttttcagttaccaagtcgactcaaaatttaaccgtaagatctctgatgcaacaatttatgcgacgaggcaggccaaggtaggtaaggataaggctgacgaaacctacgtgctcggaaaaatcaaagatatttacactcaagatggatatgatgcattcatgaagatatataaaaaggacaaatctaaattccttatgtatcgtcatgacccacagacattcgagaaagttattgagcctatcctggagaactatccgaacaagcaaataaatgagaagggcaaagaagttccatgtaatccgttcctaaagtacaaggaggaacacggatatattagaaaatacagcaaaaagggcaacggcccagaaatcaaaagccttaagtactacgatagtaaactaggaaaccacatcgacattacaccaaaagactctaataataaggtcgtactgcaaagcgtttccccatggcgcgccgatgtgtattttaataagacaacagggaagtacgaaatcttggggttaaaatatgcggatctgcaattcgaaaagggaaccggcacatacaaaatttctcaagaaaagtacaacgacataaagaagaaggaaggggtcgattctgattctgaattcaagttcacactctataagaatgatcttctgctcgtcaaggacacagagacaaaggagcagcagttgttcaggttcttgtctagaactatgccaaaacaaaagcactacgttgaactgaagccttacgataagcaaaaattcgaggggggcgaggcgcttataaaggtcctaggaaatgttgcaaactctgggcagtgtaagaagggcctgggcaagagcaacattagcatctataaggttcgaacggatgtgcttgggaaccagcatatcatcaaaaacgagggagataaaccaaagctggacttctag

SEQ ID NO: 69 - protein, 1121 aa, Streptococcus thermophilus
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 70 - DNA, 188 bp, synthetic construct
  source 1..188: mol_type = other DNA; note = Recombinant
gacattctccccaaaatatagttattgtactctcaagatttatttttccaaaagggttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 71 - DNA, 20 bp, Zea mays
  source 1..20: mol_type = unassigned DNA
gacattctccccaaaatata

SEQ ID NO: 72 - DNA, 29 bp, Zea mays
  source 1..29: mol_type = genomic DNA
gtttatctttcatgagctttttagagaat

SEQ ID NO: 73 - DNA, 29 bp, Zea mays
  source 1..29: mol_type = genomic DNA
gtaatactctatggtctgtaaggtagaat

SEQ ID NO: 74 - DNA, 28 bp, Zea mays
  source 1..28: mol_type = genomic DNA
gagatccaacgtgttgggactctagaaa

SEQ ID NO: 75 - DNA, 27 bp, Zea mays
  source 1..27: mol_type = genomic DNA
gacattctccccaaaatatacgagaaa

SEQ ID NO: 76 - DNA, 103 bp, synthetic construct
  source 1..103: mol_type = other DNA; note = Recombinant
gccggccagcatttgaaacagttttagagctagaaatagcaagttaaaataaggctagtccgttatcaacttgaaaaagtggcaccgagtcggtgcttttttt

SEQ ID NO: 77 - DNA, 103 bp, synthetic construct
  source 1..103: mol_type = other DNA; note = Recombinant
gcgcaagggatcagtaattcgttttagagctagaaatagcaagttaaaataaggctagtccgttatcaacttgaaaaagtggcaccgagtcggtgcttttttt

SEQ ID NO: 78 - DNA, 103 bp, synthetic construct
  source 1..103: mol_type = other DNA; note = Recombinant
gttatcaatttactttcaatgttttagagctagaaatagcaagttaaaataaggctagtccgttatcaacttgaaaaagtggcaccgagtcggtgcttttttt

SEQ ID NO: 79 - RNA, 103 nt, synthetic construct
  source 1..103: mol_type = other RNA; note = Recombinant
gccggccagcatttgaaacagttttagagctagaaatagcaagttaaaataaggctagtccgttatcaacttgaaaaagtggcaccgagtcggtgcttttttt

SEQ ID NO: 80 - RNA, 103 nt, synthetic construct
  source 1..103: mol_type = other RNA; note = Recombinant
gcgcaagggatcagtaattcgttttagagctagaaatagcaagttaaaataaggctagtccgttatcaacttgaaaaagtggcaccgagtcggtgcttttttt

SEQ ID NO: 81 - RNA, 103 nt, synthetic construct
  source 1..103: mol_type = other RNA; note = Recombinant
gttatcaatttactttcaatgttttagagctagaaatagcaagttaaaataaggctagtccgttatcaacttgaaaaagtggcaccgagtcggtgcttttttt

SEQ ID NO: 82 - DNA, 103 bp, synthetic construct
  source 1..103: mol_type = other DNA; note = Recombinant
gtacaaaccaactcggcaatgttttagagctagaaatagcaagttaaaataaggctagtccgttatcaacttgaaaaagtggcaccgagtcggtgcttttttt

SEQ ID NO: 83 - DNA, 103 bp, synthetic construct
  source 1..103: mol_type = other DNA; note = Recombinant
gatatgccaaacggtacttggttttagagctagaaatagcaagttaaaataaggctagtccgttatcaacttgaaaaagtggcaccgagtcggtgcttttttt

SEQ ID NO: 84 - DNA, 103 bp, synthetic construct
  source 1..103: mol_type = other DNA; note = Recombinant
ggatctcctatcataacgttgttttagagctagaaatagcaagttaaaataaggctagtccgttatcaacttgaaaaagtggcaccgagtcggtgcttttttt

SEQ ID NO: 85 - RNA, 103 nt, synthetic construct
  source 1..103: mol_type = other RNA; note = Recombinant
gtacaaaccaactcggcaatgttttagagctagaaatagcaagttaaaataaggctagtccgttatcaacttgaaaaagtggcaccgagtcggtgcttttttt

SEQ ID NO: 86 - RNA, 103 nt, synthetic construct
  source 1..103: mol_type = other RNA; note = Recombinant
gatatgccaaacggtacttggttttagagctagaaatagcaagttaaaataaggctagtccgttatcaacttgaaaaagtggcaccgagtcggtgcttttttt

SEQ ID NO: 87 - RNA, 103 nt, synthetic construct
  source 1..103: mol_type = other RNA; note = Recombinant
ggatctcctatcataacgttgttttagagctagaaatagcaagttaaaataaggctagtccgttatcaacttgaaaaagtggcaccgagtcggtgcttttttt

SEQ ID NO: 88 - DNA, 190 bp, synthetic construct
  source 1..190: mol_type = other DNA; note = Recombinant
gtttatctttcatgagctttttgttattgtactctcaagatttatttttccaaaagggttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 89 - DNA, 190 bp, synthetic construct
  source 1..190: mol_type = other DNA; note = Recombinant
gtaatactctatggtctgtaaggttattgtactctcaagatttatttttccaaaagggttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 90 - DNA, 189 bp, synthetic construct
  source 1..189: mol_type = other DNA; note = Recombinant
gagatccaacgtgttgggactgttattgtactctcaagatttatttttccaaaagggttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 91 - DNA, 188 bp, synthetic construct; identical to SEQ ID NO: 70
  source 1..188: mol_type = other DNA; note = Recombinant

SEQ ID NO: 92 - RNA, 190 nt, synthetic construct
  source 1..190: mol_type = other RNA; note = Recombinant
gtttatctttcatgagctttttgttattgtactctcaagatttatttttccaaaagggttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 93 - RNA, 190 nt, synthetic construct
  source 1..190: mol_type = other RNA; note = Recombinant
gtaatactctatggtctgtaaggttattgtactctcaagatttatttttccaaaagggttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 94 - RNA, 189 nt, synthetic construct
  source 1..189: mol_type = other RNA; note = Recombinant
gagatccaacgtgttgggactgttattgtactctcaagatttatttttccaaaagggttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 95 - RNA, 188 nt, synthetic construct
  source 1..188: mol_type = other RNA; note = Recombinant
gacattctccccaaaatatagttattgtactctcaagatttatttttccaaaagggttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 96 - protein, 1368 aa, Streptococcus pyogenes [sequence omitted]

SEQ ID NO: 97 - DNA, 4107 bp, synthetic construct
  source 1..4107: mol_type = other DNA; note = Recombinant
atggacaagaagtacagcattggtctggacatcggaacgaactcggtcggctgggccgtgattactgacgagtacaaagttcccagcaaaaaattcaaggttctaggcaacacagaccgccactcgattaaaaagaatctaatcggcgcgcttctgttcgactctggtgaaacggccgaggccacacgcttaaagaggaccgcgcgccgccgctacacgcggcgcaagaaccgaatctgttacctccaggagatcttcagtaatgagatggctaaggtcgatgacagcttcttccacaggcttgaagagtcctttctggtcgaagaggacaaaaaacacgaacgtcacccaatcttcgggaacattgtggatgaagtcgcataccacgagaagtatcctacgatctatcacctccgcaagaagctcgtggatagtaccgacaaagccgacctgcgcttaatctaccttgcgctcgcgcacatgattaagttccgaggacacttccttattgagggtgatctgaatccggacaattccgatgtggataaactgttcattcagttggtccagacatacaatcagctattcgaggagaatccgatcaatgcttccggcgtggacgcaaaggctattctgtcagcaagactttcaaagagcagaaggttggagaatctgatcgcacaacttcccggagagaagaagaatgggctcttcggcaacctcattgcgctgtctttgggtctgacaccgaactttaagtctaacttcgacctcgctgaggatgctaaacttcagcttagcaaagacacctatgatgatgacctggacaacctcctcgcccagattggagaccagtacgcggatctattcttggctgccaagaacctgtccgatgcgattctgcttagtgacatcctccgagtgaacactgaaattacgaaagcacccttgtcggctagtatgattaagcgatacgatgagcaccatcaagacctgacattgctaaaggcgctcgtaagacagcaacttcctgagaagtacaaggagatattttttgatcagtctaagaatggctacgctggttacatcgacggtggagctagtcaggaggaattctataaattcatcaagcctatcctggaaaaaatggacggtacggaggaattgctcgttaaactaaatcgagaggatctgctgagaaagcagcggactttcgacaatggttctattccgcatcagattcacctcggagaacttcacgccatcctgagacgacaggaggacttctaccctttcctgaaagacaaccgggaaaaaatcgagaagatcctgacattcaggattccttactatgtaggccctttagcgagaggcaacagtagattcgcctggatgaccagaaagtctgaggaaacaatcacaccgtggaacttcgaggaagtggttgataagggtgctagtgcccaatcattcattgagagaatgacgaacttcgacaagaatctgcctaacgagaaggttctccctaaacatagcttgctttacgagtatttcacggtgtacaatgagctaacgaaggtcaagtatgtcacagagggaatgcggaaaccggctttcctttcgggtgaacagaagaaagcaattgtggatttgctcttcaagacaaaccgaaaggtgacagtgaagcagctaaaggaggactacttcaaaaaaatagagtgcttcgactcagttgagatcagcggagtggaggaccggtttaacgcttccctcggcacttaccacgacttgctcaagatcatcaaggacaaagacttccttgataacgaggagaacgaagacatccttgaggacattgtgctgacattgacgttgttcgaggatcgggagatgatcgaggaacgcctcaagacgtacgcccatctgttcgatgataaggtgatgaagcagttaaagaggagacgttacactggctggggccgtctctctcgcaaactgataaacgggataagggataaacaaagcggaaagacaatcctcgatttccttaaatccgacggcttcgctaaccggaacttcatgcagctcatccatgatgactcactgacgttcaaggaggacatccagaaagctcaagtgtctggccagggtgacagcttgcacgagcacatcgcaaatctagccggttcaccggcgataaagaagggcattctacaaacggtgaaagtggtggacgagcttgtgaaggtcatgggtcgccataagccagagaacattgttatcgaaatggcgagggagaaccagacaacgcagaagggacaaaaaaacagtagggagcggatgaagcgcatcgaggaaggcattaaggaacttggaagccaaatcctgaaagagcacccggtggagaatacgcagttgcagaacgagaaactgtacctctactacttgcagaatggacgtgatatgtatgtggatcaagagttggacatcaaccgattgtctgactatgacgtggatcacatagtaccacagtccttcctcaaggatgatagcatagacaacaaggttcttactcgtagcgacaagaatcgtggcaaatcggacaatgttccatctgaggaagttgtcaaaaagatgaaaaattattggaggcaacttctgaacgcgaagctaattacacaaaggaaattcgacaatctcactaaggccgagagaggagggttaagtgagttagacaaggctggcttcatcaagcggcagttggtcgagactcgtcagattactaagcacgtggctcagatcctggattcgcgcatgaacaccaaatacgacgagaatgacaaactcatccgtgaagttaaggtgattacactgaaatccaagctggtctctgactttaggaaagacttccaattctacaaggtgagagagattaacaactaccaccacgcgcatgacgcctacttgaatgctgtggttgggactgccctgataaagaaatatcctaaacttgagtctgagttcgtttacggtgactacaaggtttatgacgttaggaagatgatcgccaaatccgaacaggagattgggaaagcaactgccaaatatttcttttactccaacattatgaactttttcaagacagaaatcacactcgccaatggcgagattcggaagagaccactaatcgaaacaaacggcgaaactggtgaaatcgtttgggataagggtagggacttcgcaactgttaggaaggtcttgtcgatgcctcaagtgaacatagtcaaaaaaacagaggtccagaccggtgggttctcaaaggagtctattctgccaaagcgtaacagcgacaaactcatcgctcgcaaaaaggactgggatcctaaaaagtacggtggattcgacagcccgaccgttgcttattctgttctcgtagttgctaaagtcgagaagggcaagtccaaaaaactcaaatcggttaaggaactgctcggaatcacgataatggaacgaagcagtttcgaaaaaaatccaattgacttcctggaagctaaaggttacaaggaggtcaagaaggatcttatcatcaagctacccaagtacagtctgttcgaactggagaacggtcgcaagagaatgctggcctcggctggtgaactccagaagggcaatgagctggccctgccgtccaagtacgtgaactttctgtacctggcatctcattacgagaagctcaagggctcaccagaggacaacgagcagaagcagttgttcgttgaacagcacaaacactatcttgatgagatcattgagcaaattagcgagttcagtaagcgagttattctggctgatgctaacctggataaggtgctctctgcctacaacaagcaccgggataaacctattagggaacaggcggagaacataatccacctcttcactctgacgaatctcggcgcgcctgcggccttcaaatattttgataccaccatcgacaggaagcgctacacaagcactaaagaggtgctggacgccactctcattcaccagtctattaccgggctctacgagacacggattgacctctcccagctaggtggcgattaa

SEQ ID NO: 98 - DNA, 469 bp, synthetic construct
  source 1..469: mol_type = other DNA; note = Recombinant
cgtctttgtatcggaatataaatttatcactattttatgataaagtaaatctgtttccctgtagagttaattaattaatgtaagtataagcgtaatttatagggcactagtaggactgtcgactgtgcgctcggcccggataatgcgtcaaaagcgaagacgtgcacgtgggatgggaaaacacgaagcgtggtctgctttttcgcatgatatctgggccgcaccaaagaatccagcccacgcggcgtggcgccgtcgttacttgcgggggaaggaaacgagggacgaaccgagatttagcaccagaccggccagcgagcattgcagacaccggcttataagttcagctgcgactaccactccgtctcttcggagacatccgataaaattggaacgatacagagaagattagcatggcccctgcgcaaggatgacacgcacaaatcgagaaatggtccaaatttttttg

SEQ ID NO: 99 - DNA, 268 bp, synthetic construct
  source 1..268: mol_type = other DNA; note = Recombinant
cctgttccgctttgctagcttgcgccctgactgtccagcccacgcgcttcggtccgattctgctaggctggtgcaagcgagccgagacttttttttagaaccaccttgctcagcaaaccttaggaacaccggcttataagtcgaagcgaagcgctgtgcactgtctcttcggagacatccgataaaattggaacgatacagagaagattagcatggcccctgcgcaaggatgacacgcacaaatcgagaaatggtccaaatttttttg

SEQ ID NO: 100 - DNA, 426 bp, synthetic construct
  source 1..426: mol_type = other DNA; note = Recombinant
gccaggatttatacaactattttcttatttatttctttaacattttcccttctacgcacaataggagataatgtcaagcgttgacggtgcacatatatttgtttttttaaaggcgtagtggcgtgtgtgcaaaaacatcctcacaggaaagacacgaagaaacatggtcaatggcccattatataaagcaccgccacaaagcccaaataccagttcgttggagcaagtaacgcgctaggcaacaggcaaacagtttgtcccacctcgtccagtcacaaaggcaaagcgtgacttataagccagagcggaagaaccataccgtctcttcggagacatccgataaaattggaacgatacagagaagattagcatggcccctgcgcaaggatgacacgcacaaatcgagaaatggtccaaatttttttg

SEQ ID NO: 101 - DNA, 469 bp, synthetic construct
  source 1..469: mol_type = other DNA; note = Recombinant
ggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagttaggtggagcaaagcgctgggtaatacgcaaacgttttgtcccaccttgactaatcacaagagtggagcgtaccttataaaccgagccgcaagcaccgaattgtctcttcggagacatccgataaaattggaacgatacagagaagattagcatggcccctgcgcaaggatgacacgcacaaatcgagaaatggtccaaatttttttg

SEQ ID NO: 102 - DNA, 80 bp, synthetic construct
  source 1..80: mol_type = other DNA; note = Recombinant
acacttaatcggctctcaagaagtcctcaagggataacagggtaatatagcgtaactataacggtcctaaggtagcgaat

SEQ ID NO: 103 - DNA, 80 bp, synthetic construct; identical to SEQ ID NO: 102
  source 1..80: mol_type = other DNA; note = Recombinant

SEQ ID NO: 104 - DNA, 59 bp, synthetic construct
  source 1..59: mol_type = other DNA; note = Recombinant
acacttaatcggctctcaagaagtcctcaagtaactataacggtcctaaggtagcgaat

SEQ ID NO: 105-106 - skipped (2 entries)

SEQ ID NO: 107 - DNA, 155 bp, synthetic construct
  source 1..155: mol_type = other DNA; note = Recombinant
gtttatctttcatgagctttttgttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 108 - DNA, 155 bp, synthetic construct
  source 1..155: mol_type = other DNA; note = Recombinant
gtaatactctatggtctgtaaggttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 109 - DNA, 154 bp, synthetic construct
  source 1..154: mol_type = other DNA; note = Recombinant
gagatccaacgtgttgggactgttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 110 - DNA, 153 bp, synthetic construct
  source 1..153: mol_type = other DNA; note = Recombinant
gacattctccccaaaatatagttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 111 - RNA, 155 nt, synthetic construct
  source 1..155: mol_type = other RNA; note = Recombinant
gtttatctttcatgagctttttgttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 112 - RNA, 155 nt, synthetic construct
  source 1..155: mol_type = other RNA; note = Recombinant
gtaatactctatggtctgtaaggttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 113 - RNA, 154 nt, synthetic construct
  source 1..154: mol_type = other RNA; note = Recombinant
gagatccaacgtgttgggactgttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 114 - RNA, 153 nt, synthetic construct
  source 1..153: mol_type = other RNA; note = Recombinant
gacattctccccaaaatatagttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 115 - DNA, 90 bp, synthetic construct
  source 1..90: mol_type = other DNA; note = Recombinant
ttaagggataacagggtaatatagcgtaactataacggtcctaaggtagcgaattacgatacaaggctacctagcttcgcagttacgcta

SEQ ID NO: 116 - DNA, 90 bp, synthetic construct
  source 1..90: mol_type = other DNA; note = Recombinant
tagcgtaactgcgaagctaggtagccttgtatcgtaattcgctaccttaggaccgttatagttacgctatattaccctgttatcccttaa

SEQ ID NO: 117 - DNA, 20 bp, synthetic construct
  source 1..20: mol_type = other DNA; note = Recombinant
ctatattaccctgttatccc

SEQ ID NO: 118 - protein, 1388 aa, synthetic construct
  source 1..1388: note = Recombinant
MGSKKRRIKQDDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSKKRRIKQD

SEQ ID NO: 119 - DNA, 4542 bp, synthetic construct
  source 1..4542: mol_type = other DNA; note = Recombinant
atgggatctaagaagagaagaattaaacaagatgacaagaagtacagcattggtctggacatcggaacgaactcggtcggctgggccgtgattactgacgagtacaaagttcccagcaaaaaattcaaggttctaggcaacacagaccgccactcgattaaaaagaatctaatcggtgcgcttctgttcgactctggtgaaacggccgaggccacacgcttaaagaggaccgcgcgccgccgctacacgcggcgcaagaaccgaatctgttacctccaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggagatcttcagtaatgagatggctaaggtcgatgacagcttcttccacaggcttgaagagtcctttctggtcgaagaggacaaaaaacacgaacgtcacccaatcttcgggaacattgtggatgaagtcgcataccacgagaagtatcctacgatctatcacctccgcaagaagctcgtggatagtaccgacaaagccgacctgcgcttaatctaccttgcgctcgcgcacatgattaagttccgaggacacttccttattgagggtgatctgaatccggacaattccgatgtggataaactgttcattcagttggtccagacatacaatcagctattcgaggagaatccgatcaatgcttccggcgtggacgcaaaggctattctgtcagcaagactttcaaagagcagaaggttggagaatctgatcgcacaacttcccggagagaagaagaatgggctcttcggcaacctcattgcgctgtctttgggtctgacaccgaactttaagtctaacttcgacctcgctgaggatgctaaacttcagcttagcaaagacacctatgatgatgacctggacaacctcctcgcccagattggagaccagtacgcggatctattcttggctgccaagaacctgtccgatgcgattctgcttagtgacatcctccgagtgaacactgaaattacgaaagcacccttgtcggctagtatgattaagcgatacgatgagcaccatcaagacctgacattgctaaaggcgctcgtaagacagcaacttcctgagaagtacaaggagatattttttgatcagtctaagaatggctacgctggttacatcgacggtggagctagtcaggaggaattctataaattcatcaagcctatcctggaaaaaatggacggtacggaggaattgctcgttaaactaaatcgagaggatctgctgagaaagcagcggactttcgacaatggttctattccgcatcagattcacctcggagaacttcacgccatcctgagacgacaggaggacttctaccctttcctgaaagacaaccgggaaaaaatcgagaagatcctgacattcaggattccttactatgtaggccctttagcgagaggcaacagtagattcgcctggatgaccagaaagtctgaggaaacaatcacaccgtggaacttcgaggaagtggttgataagggtgctagtgcccaatcattcattgagagaatgacgaacttcgacaagaatctgcctaacgagaaggttctccctaaacatagcttgctttacgagtatttcacggtgtacaatgagctaacgaaggtcaagtatgtcacagagggaatgcggaaaccggctttcctttcgggtgaacagaagaaagcaattgtggatttgctcttcaagacaaaccgaaaggtgacagtgaagcagctaaaggaggactacttcaaaaaaatagagtgcttcgactcagttgagatcagcggagtggaggaccggtttaacgcttccctcggcacttaccacgacttgctcaagatcatcaaggacaaagacttccttgataacgaggagaacgaagacatccttgaggacattgtgctgacattgacgttgttcgaggatcgggagatgatcgaggaacgcctcaagacgtacgcccatctgttcgatgataaggtgatgaagcagttaaagaggagacgttacactggctggggccgtctctctcgcaaactgataaacgggataagggataaacaaagcggaaagacaatcctcgatttccttaaatccgacggcttcgctaaccggaacttcatgcagctcatccatgatgactcactgacgttcaaggaggacatccagaaagctcaagtgtctggccagggtgacagcttgcacgagcacatcgcaaatctagccggttcaccggcgataaagaagggcattctacaaacggtgaaagtggtggacgagcttgtgaaggtcatgggtcgccataagccagagaacattgttatcgaaatggcgagggagaaccagacaacgcagaagggacaaaaaaacagtagggagcggatgaagcgcatcgaggaaggcattaaggaacttggaagccaaatcctgaaagagcacccggtggagaatacgcagttgcagaacgagaaactgtacctctactacttgcagaatggacgtgatatgtatgtggatcaagagttggacatcaaccgattgtctgactatgacgtggatcacatagtaccacagtccttcctcaaggatgatagcatagacaacaaggttcttactcgtagcgacaagaatcgtggcaaatcggacaatgttccatctgaggaagttgtcaaaaagatgaaaaattattggaggcaacttctgaacgcgaagctaattacacaaaggaaattcgacaatctcactaaggccgagagaggagggttaagtgagttagacaaggctggcttcatcaagcggcagttggtcgagactcgtcagattactaagcacgtggctcagatcctggattcgcgcatgaacaccaaatacgacgagaatgacaaactcatccgtgaagttaaggtgattacactgaaatccaagctggtctctgactttaggaaagacttccaattctacaaggtgagagagattaacaactaccaccacgcgcatgacgcctacttgaatgctgtggttgggactgccctgataaagaaatatcctaaacttgagtctgagttcgtttacggtgactacaaggtttatgacgttaggaagatgatcgccaaatccgaacaggagattgggaaagcaactgccaaatatttcttttactccaacattatgaactttttcaagacagaaatcacactcgccaatggcgagattcggaagagaccactaatcgaaacaaacggcgaaactggtgaaatcgtttgggataagggtagggacttcgcaactgttaggaaggtcttgtcgatgcctcaagtgaacatagtcaaaaaaacagaggtccagaccggtgggttctcaaaggagtctattctgccaaagcgtaacagcgacaaactcatcgctcgcaaaaaggactgggatcctaaaaagtacggtggattcgacagcccgaccgttgcttattctgttctcgtagttgctaaagtcgagaagggcaagtccaaaaaactcaaatcggttaaggaactgctcggaatcacgataatggaacgaagcagtttcgaaaaaaatccaattgacttcctggaagctaaaggttacaaggaggtcaagaaggatcttatcatcaagctacccaagtacagtctgttcgaactggagaacggtcgcaagagaatgctggcctcggctggtgaactccagaagggcaatgagctggccctgccgtccaagtacgtgaactttctgtacctggcatctcattacgagaagctcaagggctcaccagaggacaacgagcagaagcagttgttcgttgaacagcacaaacactatcttgatgagatcattgagcaaattagcgagttcagtaagcgagttattctggctgatgctaacctggataaggtgctctctgcctacaacaagcaccgggataaacctattagggaacaggcggagaacataatccacctcttcactctgacgaatctcggcgcgcctgcggccttcaaatattttgataccaccatcgacaggaagcgctacacaagcactaaagaggtgctggacgccactctcattcaccagtctattaccgggctctacgagacacggattgacctctcccagctaggtggcgatggatctaagaagagaagaattaaacaagat

SEQ ID NO: 120 - protein, 10 aa, synthetic construct
  source 1..10: note = Recombinant
GSKKRRIKQD

SEQ ID NO: 121 - DNA, 92 bp, synthetic construct
  source 1..92: mol_type = other DNA; note = Recombinant
caagggataacagggtaatatagcgtaactataacggtcctaaggtagcgaattacgatacaaggctacctagcttcgcagttacgctagta

SEQ ID NO: 122 - DNA, 92 bp, synthetic construct
  source 1..92: mol_type = other DNA; note = Recombinant
tactagcgtaactgcgaagctaggtagccttgtatcgtaattcgctaccttaggaccgttatagttacgctatattaccctgttatcccttg

SEQ ID NO: 123 - DNA, 73 bp, synthetic construct
  source 1..73: mol_type = other DNA; note = Recombinant
catgccctcttaggcagtagccggccagcatttgaattaagggataacagggtaatatagcgtaactataacg

SEQ ID NO: 124 - DNA, 73 bp, synthetic construct; identical to SEQ ID NO: 123
  source 1..73: mol_type = other DNA; note = Recombinant

SEQ ID NO: 125 - DNA, 68 bp, synthetic construct
  source 1..68: mol_type = other DNA; note = Recombinant
catgccctcttaggcagtagccggccagcatttaagggataacagggtaatatagcgtaactataacg

SEQ ID NO: 126 - DNA, 73 bp, synthetic construct
  source 1..73: mol_type = other DNA; note = Recombinant
aaagcaacacttaatcggctctcaagaagtcctcaagggataacagggtaatatagcgtaactataacggtcc

SEQ ID NO: 127 - DNA, 73 bp, synthetic construct; identical to SEQ ID NO: 126
  source 1..73: mol_type = other DNA; note = Recombinant

SEQ ID NO: 128 - DNA, 52 bp, synthetic construct
  source 1..52: mol_type = other DNA; note = Recombinant
aaagcaacacttaatcggctctcaagaagtcctcaagtaactataacggtcc

SEQ ID NO: 129 - DNA, 73 bp, synthetic construct; identical to SEQ ID NO: 126
  source 1..73: mol_type = other DNA; note = Recombinant

SEQ ID NO: 130 - DNA, 71 bp, synthetic construct
  source 1..71: mol_type = other DNA; note = Recombinant
aaagcaacacttaatcggctctcaagaagtcctcaagggataacagggtaatatagcgtaactataagtcc

SEQ ID NO: 131 - DNA, 69 bp, synthetic construct
  source 1..69: mol_type = other DNA; note = Recombinant
aaagcaacacttaatcggctctcaagaagtcctcaagataacagggtaatatagcgtaactataagtcc

SEQ ID NO: 132 - DNA, 18 bp, synthetic construct
  source 1..18: mol_type = other DNA; note = Recombinant
gaaacggttcggcatgca

SEQ ID NO: 133 - DNA, 15 bp, synthetic construct
  source 1..15: mol_type = other DNA; note = Recombinant
tgagctggcacgaac

SEQ ID NO: 134 - DNA, 20 bp, synthetic construct
  source 1..20: mol_type = other DNA; note = Recombinant
ccctgtcgtccgtccaagta

SEQ ID NO: 135 - protein, 1142 aa, synthetic construct
  source 1..1142: note = Recombinant
MGSKKRRIKQDMSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDFGSKKRRIKQD

SEQ ID NO: 136 - DNA, 3618 bp, synthetic construct
  source 1..3618: mol_type = other DNA; note = Recombinant
atgggatctaagaagagaagaattaaacaagatatgagtgacctggtgctagggttggatataggcattggctccgtgggggttggcattcttaataaggtgaccggcgaaataattcataaaaactcacgcatctttccagcagcccaggctgagaacaatctggtccgtagaaccaaccggcagggtcgaaggttagccaggcgcaagaagcacagacgggtccggctcaacaggcttttcgaggagtctggtttgatcaccgatttcactaagatttctatcaacctgaatccttatcagctgcgcgttaaaggtctcacagacgaacttagcaacgaagagttgttcatcgccctgaaaaatatggtcaagcatcgcggcattagctacctggacgacgcttcggatgatggcaacagtagtgtaggtgactacgctcagatcgtgaaagagaactcgaagcaattggagaccaagaccccgggccaaattcaactcgaaaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtaccagacgtatggacagttacgaggcgattttaccgttgaaaaggatggtaagaagcacaggctgattaatgtgtttccgacctcagcttatcgctctgaggcgctgcgtattttgcagacccaacaggaatttaacccgcaaataacggacgagttcataaaccgatacttagagattcttacaggtaaacgtaaatactatcacggcccaggaaatgaaaagtccaggacagattatggtcgatatcgcacttccggagagactctcgacaatatctttggcattcttataggcaaatgtaccttctaccctgacgaatttagagcagcgaaggcttcatatacagcacaagagtttaatcttctcaacgacctcaacaacttgactgtgcctactgaaaccaaaaagcttagcaaggagcaaaaaaatcaaatcattaactatgttaagaatgagaaagctatggggcccgcaaaattgttcaagtacatagctaagttacttagctgtgacgttgctgatattaagggttaccgtattgacaagtctggtaaagctgaaattcacacctttgaggcttataggaagatgaagacccttgagacacttgacattgagcagatggatagggagactttggacaaactggcatacgtcttgacattgaacaccgaaagggaaggcatccaggaagctctggaacatgaatttgcagatggttcgttcagccaaaaacaggttgacgagctggtccaatttagaaaggcaaactcaagcatattcggtaaaggttggcacaacttcagcgttaagctgatgatggaactcattccagaattatatgaaacctctgaggaacagatgacgattctcacaagattgggtaagcagaaaacaaccagctctagcaataagactaaatacattgacgaaaagctcctcaccgaagagatttataacccggtcgtggcaaagagtgtacggcaagccatcaagatcgttaatgccgctatcaaggagtatggtgattttgataatattgtgattgaaatggcacgcgagactaacgaggacgacgagaagaaagctatacagaagattcaaaaggctaataaggacgagaaggacgccgcaatgctaaaggcggccaatcaatataatgggaaggctgaactacctcatagcgtcttccatggacataagcaattagcaactaaaataagattatggcaccagcaaggcgaacggtgtctttatacaggtaaaacgatatctattcacgacctgattaacaactctaaccagtttgaagtggatcatatcttaccactaagtatcaccttcgacgattcacttgctaacaaggtgctcgtttacgccactgcgaaccaagagaaagggcagaggactccataccaggcccttgacagcatggacgacgcctggagttttagggaattaaaagctttcgtacgtgagtcaaagacgctttcaaataaaaaaaaggagtacttgctcactgaagaagacatctcaaaattcgacgtgcgcaaaaaattcattgagcggaacttagtcgacactcggtacgcatcaagagtagtgttgaacgccctccaggagcactttagggcacataagatcgacaccaaggtttcagttgttaggggtcagtttacatcgcagcttagacgccattggggtatagaaaaaacacgtgatacctaccatcaccatgcagttgacgctctcatcattgcagcatcttctcaacttaatttgtggaaaaagcaaaagaacactctggtctcatatagcgaagatcagctgcttgatattgaaaccggcgagctgatttctgacgacgaatacaaagaatctgtgtttaaggcaccatatcaacactttgtagacacgcttaaatctaaagagtttgaggattcgatccttttcagttaccaagtcgactcaaaatttaaccgtaagatctctgatgcaacaatttatgcgacgaggcaggccaaggtaggtaaggataaggctgacgaaacctacgtgctcggaaaaatcaaagatatttacactcaagatggatatgatgcattcatgaagatatataaaaaggacaaatctaaattccttatgtatcgtcatgacccacagacattcgagaaagttattgagcctatcctggagaactatccgaacaagcaaataaatgagaagggcaaagaagttccatgtaatccgttcctaaagtacaaggaggaacacggatatattagaaaatacagcaaaaagggcaacggcccagaaatcaaaagccttaagtactacgatagtaaactaggaaaccacatcgacattacaccaaaagactctaataataaggtcgtactgcaaagcgtttccccatggcgcgccgatgtgtattttaataagacaacagggaagtacgaaatcttggggttaaaatatgcggatctgcaattcgaaaagggaaccggcacatacaaaatttctcaagaaaagtacaacgacataaagaagaaggaaggggtcgattctgattctgaattcaagttcacactctataagaatgatcttctgctcgtcaaggacacagagacaaaggagcagcagttgttcaggttcttgtctagaactatgccaaaacaaaagcactacgttgaactgaagccttacgataagcaaaaattcgaggggggcgaggcgcttataaaggtcctaggaaatgttgcaaactctgggcagtgtaagaagggcctgggcaagagcaacattagcatctataaggttcgaacggatgtgcttgggaaccagcatatcatcaaaaacgagggagataaaccaaagctggacttcggatctaagaagagaagaattaaacaagattag

SEQ ID NO: 137 - DNA, 26 bp, synthetic construct
  source 1..26: mol_type = other DNA; note = Recombinant
ccttgtatcgtaattcgctaccttag

SEQ ID NO: 138 - DNA, 20 bp, synthetic construct
  source 1..20: mol_type = other DNA; note = Recombinant
ctatattaccctgttatccc

SEQ ID NO: 139 - DNA, 20 bp, synthetic construct
  source 1..20: mol_type = other DNA; note = Recombinant
taatcggctctcaagaagtc

SEQ ID NO: 140 - DNA, 20 bp, synthetic construct
  source 1..20: mol_type = other DNA; note = Recombinant
gggactctagaaaaaacttg

SEQ ID NO: 141 - DNA, 21 bp, synthetic construct
  source 1..21: mol_type = other DNA; note = Recombinant
taaggagatgagtttgagacc

SEQ ID NO: 142 - DNA, 20 bp, synthetic construct
  source 1..20: mol_type = other DNA; note = Recombinant
aacatttcagtaatcacgag

SEQ ID NO: 143 - DNA, 20 bp, synthetic construct
  source 1..20: mol_type = other DNA; note = Recombinant
taatatgatggcatgccctc

SEQ ID NO: 144 - DNA, 68 bp, synthetic construct
  source 1..68: mol_type = other DNA; note = Recombinant
ctatctagtgaagatgtaatactctatggtctgtttaagggataacagggtaatatagcgtaactata

SEQ ID NO: 145 - DNA, 55 bp, synthetic construct
  source 1..55: mol_type = other DNA; note = Recombinant
ctatctagtgaagatgtaatactctatggtctgtgggtaatatagcgtaactata

SEQ ID NO: 146 - DNA, 397 bp, Solanum lycopersicum
  source 1..397: mol_type = genomic DNA
aataaatatattatctatcattagaacttgaattataagtgaataatagattattttttgtaatatgaattaaaagtgtattaaacatgtattaacggtgatcaattggttaaaaaaaagtttattattaaaatgataaatctttttaatttatagtatatttatgtaagttttcacgttgagtaaatagcgaagaagttgggcccaaccaagtaaaataagaaggccgggccattacaattaagtcgtcacacaactgggcttcattgaaaaaagcgcaaaaccgattccaggcccgtgttagcatgaagactcaactcaaccagagatttctccctcatcgcttacagaaaaaagctatatgctgtttatattgcgaaatctaacagtgtagttt

SEQ ID NO: 147 - DNA, 397 bp, Solanum lycopersicum
  source 1..397: mol_type = genomic DNA
tacttatgagaagaagtaactgatttaaaattttcactaatagggttcgaaaaatgaaaatgtaatacgtggaacttgaatgtaaaacctcaaggaattcttgtgtttaagaaattcaaaatctctctaaatgtatacaaaagatgatttctttttaccttatatatagtaaaataaaattgtcggataaattcgagtgaacaccctagcaccccctaaatcctcccccgtagtcggcccattacagttaaagtccaggtacaacaaaatgggcttcgattaagatggaataaaaggagtccaggcccatgagcccaacaaacaagctatttctccctcatcggcgcacaaagaagctttattctcttattatagctgaatattagcatgtgtgttt

SEQ ID NO: 148 - DNA, 397 bp, Solanum lycopersicum
  source 1..397: mol_type = genomic DNA
ttctatttgtgctacatatattagacaaggaaaataacatatgttattttgaaatcacgtatatttactataaattacaatgattaacaacttaaaatatttaaatgaaaatcatattaatgactctctaaattttatctgtgtcacataaatgaaaaacaaaaaataacaaatattgtattcgcacgggcgcatgtgtctagttagttataaacgaagaaataaggggctgatttcgaaataaacgttcttagaattggaagaaatgttcagtttctaaacttgtaggactaaagcaataacttttatttaatttattttcttttatgtttctcccacatcgatcatacatataactatacagcagtataagaactctagcgaagcaataatgctc

SEQ ID NO: 149 - DNA, 261 bp, Solanum lycopersicum
  source 1..261: mol_type = genomic DNA
ataaatctttttaatttatagtatatttatgtaagttttcacgttgagtaaatagcgaagaagttgggcccaaccaagtaaaataagaaggccgggccattacaattaagtcgtcacacaactgggcttcattgaaaaaagcgcaaaaccgattccaggcccgtgttagcatgaagactcaactcaaccagagatttctccctcatcgcttacagaaaaaagctatatgctgtttatattgcgaatctaacagtgtagttt

SEQ ID NO: 150 - DNA, 103 bp, Solanum lycopersicum
  source 1..103: mol_type = unassigned DNA
ggatctttccaagcttgagggttttagagctagaaatagcaagttaaaataaggctagtccgttatcaacttgaaaaagtggcaccgagtcggtgcttttttt

SEQ ID NO: 151 - DNA, 119 bp, Solanum lycopersicum
  source 1..119: mol_type = other DNA
aagctaatcaagctgctaatactatttcaaaacttaggcattctaatcctcctcaagcttggaaagatcctttgaagaattgtgccttttcgtataaggtaatgtttattcgttcgtcg

SEQ ID NO: 152 - DNA, 120 bp, synthetic construct
  source 1..120: mol_type = other DNA; note = Recombinant
aagctaatcaagctgctaatactatttcaaaacttaggcattctaatcctcctacaagcttggaaagatcctttgaagaattgtgccttttcgtataaggtaatgtttattcgttcgtcg

SEQ ID NO: 153 - DNA, 120 bp, synthetic construct
  source 1..120: mol_type = other DNA; note = Recombinant
aagctaatcaagctgctaatactatttcaaaacttaggcattctaatcctcctgcaagcttggaaagatcctttgaagaattgtgccttttcgtataaggtaatgtttattcgttcgtcg

SEQ ID NO: 154 - DNA, 117 bp, synthetic construct
  source 1..117: mol_type = other DNA; note = Recombinant
aagctaatcaagctgctaatactatttcaaaacttaggcattctaatcctcctagcttggaaagatcctttgaagaattgtgccttttcgtataaggtaatgtttattcgttcgtcg

SEQ ID NO: 155 - DNA, 116 bp, synthetic construct
  source 1..116: mol_type = other DNA; note = Recombinant
aagctaatcaagctgctaatactatttcaaaacttaggcattctaatcctcctgcttggaaagatcctttgaagaattgtgccttttcgtataaggtaatgtttattcgttcgtcg

SEQ ID NO: 156 - DNA, 114 bp, synthetic construct
  source 1..114: mol_type = other DNA; note = Recombinant
aagctaatcaagctgctaatactatttcaaaacttaggcattctaatcctcctttggaaagatcctttgaagaattgtgccttttcgtataaggtaatgtttattcgttcgtcg

SEQ ID NO: 157 - DNA, 20 bp, synthetic construct
  source 1..20: mol_type = other DNA; note = Recombinant
tagagacaacatgcaagaac

SEQ ID NO: 158 - DNA, 20 bp, synthetic construct
  source 1..20: mol_type = other DNA; note = Recombinant
taagcaaccagtaagatagg

SEQ ID NO: 159 - DNA, 380 bp, synthetic construct
  source 1..380: mol_type = other DNA; note = Recombinant
tagagacaacatgcaagaacacaccaaattataatttgtgtgtgaaaactttgtctttagacaaaagaagtgaaaaagcaggagatattacaacattagcattaattatggttgatgctattaaatctaaagctaatcaagctgctaatactatttcaaaacttaggcattctaatcctcctcaagcttggaaagatcctttgaagaattgtgccttttcgtataaggtaatgtttattcgttcgtcgtttcaatttgtttgtcctaacaaaactcgactatgatgaattaggattttatgtttattttttctgtctcaatttgcttgtcttacttctttttttggctaaaagtttcgaccctatcttactggttgctta

SEQ ID NO: 160 - DNA, 246 bp, Glycine max
  source 1..246: mol_type = genomic DNA
ggaaacttctgtttgtccccatactccaaaaacaaaaccattttttttttatcttcgtttttgtttgctttgactgtgagttgaggcccaactttctgcttctgtccgactctatttgatgaattttgtttgcctcctgtgatgtgaaggatgtatcattgaaagggaacgtgtctcaatgatcccacatcggccaaatatgctcattacattgcgtttatatagtcccaggaaaacatatggatt

SEQ ID NO: 161 - DNA, 493 bp, Glycine max
  source 1..493: mol_type = genomic DNA
tctacaaaacattataaaaagtaagatataacaactttttttttaaaaaaatcaataggaaatattatcggttcagttaatttacagagagatatttatttcatatgtgccagaagtatttcagttccttatgaaaaatcagaaaaatgtatggaataaaatataataatcgatactaataatagaacaaaataaatggtaaaatgtcaaatcaaaactaggctgcagtatgcagagcagagtcatgatgatactacttactacaccgattcttgtgtgcagaaaaatatgttaaaataattgaatctttctctagccaaatttgacaacaatgtacaccgttcatattgagagacgatgcttcttgtttgctttcggtggaagctgcatatactcaacattactccttcagcgagttttccaactgagtcccacattgcccagacctaacacggtattcttgtttataatgaaatgtgccaccacatggatt

SEQ ID NO: 162 - DNA, 559 bp, Glycine max
  source 1..559: mol_type = genomic DNA
aggtggagttgttccagatttagttttcgacttagatgatgcatggaactggctagtgacgtggatggtggtaggttactttcaggtcatgattttttgtttctaaatgatactcacactcccttccagttttttttttttaaactcagctcccttgcttcctccaccggttatcataatactgaaccaaatcaaacattacagtcaaggtactatgaatatgaaacctgaaatcctatgaatgtcataaatttattttaaataataaatttatttagaataatatttttttgggtaagagttataaaataaaatacaaaaaaaaaacctaatatcaatttttcactgactccgtttatattgagacttgagaaagatggttcccgtttgctcccggtggaggctccgaggctgtgtatatactcgacattactttagcttgttttgttgtttctttccctttcccacaagactcaggtctcgttcgcaaacgagtcccacaccgtctaaacttaccacaatattagcgtttataattagatgcactgcatcacttatt

SEQ ID NO: 163 - DNA, 596 bp, Glycine max
  source 1..596: mol_type = genomic DNA
aaattacacggtagtacatcctattatagatcgatgaattcttaactactcgtgtcccttaggccaggcctgttttcttgcacaatttctgaaatgtatacggtttccacttcaacctttttaaccgcacaaagttttaaccagatttatataatttatttttgaatccccaatacatatcattataacatatcaattatcaaatatttcaataacctcatgatatggcaatgaatacatcttcttctcaatgaacagagatttctgaaaaagattaggaaagtgaaagcatactcgtttgcaatgtaaaactgatacttccccaaaatcatcatattccaaatatgccctggtgttactgaccaaaaccagaaaaaagaaacggaagacatatacgtctaaacggagaaatttcaaaaaacaaaaattggatcatttctcgatttgtgggtgtcatcttgtgcagggcatgctaatcttctctttaccctttcccacaagactcagcgcatgttgtctcgtctcatccaagtcccacaccgcctaaacttaacacaatattagtatttataatgacatacaacattcaagatgtt

SEQ ID NO: 164 - DNA, 737 bp, Glycine max
  source 1..737: mol_type = genomic DNA
aattatttaatcctaaaaatcaatcttacaaggaaccactataaaactagttgaacccatcgaaaactaactacagttgacaaaatctatttggttggttgattttttttaattaaaaacatccaatcttaatgatataattatagcttaatattataagatttttataaaaattatatttattttgtttctaattatgctaagagatattattatttgttatttaacttaaatattatcacaaacttgattgaaacttatgtttaatttaaaatattatatgtcatgagttatgactccaataatcacaattataaagtgaagtttaatttttagtattacaaatatttttttgttgtttaattttaattacttaatgtattatgttaataattaaaaatacaaattatttattattaatgcaatcacagtttgtggatttgacaaaagaaataggggatctaaaattgtagataagccaaagttaaaacttgaattgactattttttgctctttactctgcaccaactttactattccttcttttagtgtgagcttcatgcatcttgttcaccgcaattccgctcggtgaaagttgcacaattcactcacaatctgtttctggtctgttaggtttgttacttggagtgacacgatgacgcaacagtacaagtcccacatcgtttgagtatacagttttcaagcagtttatattcccatagccttagcaagagctt

SEQ ID NO: 165 - DNA, 484 bp, Glycine max
  source 1..484: mol_type = genomic DNA
ttttttaataaaaaaaattcaatgggagatactatggattcaattaccttactgattttatttcatatgtgccagaagtatttcagtttattttgaaaaatcagaaaaaaaatgtctggaataaaatataataagcgatactaataaataattgaacaagataaatggtaaaatgtcaaatcaaaactaggctacagagtgcagagcagagtcatgatgaatgacagctagttctacttactacaccgattcttgtgtacataaaaatattttaaaataattgaatctttctttagccagctttgacaacaatgtacaccgttcgtacttcttactggtaggcaatgcttcttgtttgctttcggtggaaggtgtatatactcaacattacttctttttcagcgtgttttcttacgggagtcccacaccgcccaaaactaatacagtattcttgtttataaagaagtgcaccacttcaattgtt

SEQ ID NO: 166 - DNA, 427 bp, Glycine max
  source 1..427: mol_type = genomic DNA
cgataaaaatgttttaaacgatatatattataaaaaaaaacgtttcaaaaataaatacaaaaatgtttttaaatatatataatttaactcattaaagaaaataaaaatgcaagtgcggtgacaagacaagctaaaagttgcaaaagaaatggcagggctataaggctcacctactcctggatttaccaaattttggttcgtccctatactcgaaaaataaaacaaaataaatttcagtatcttcgtttttgtatgctttgactgtgaggcgaggccaactttcttcttctgtctgagatgaattttgtttgcctcctgtgaaggatgtatcattcaaagtgaatgttttgcaactgccagtagtcccacatcgaccaaatattcttattacagtgtgtttatatagcacctggagaaggaatgggtt

SEQ ID NO: 167 - DNA, 400 bp, Glycine max
  source 1..400: mol_type = genomic DNA
gaaaagcattcagaatatttgagcctctaaaaacttttcttctttttctttgaggagtgtaatgtggatcccaagtaaccaagaaaagcaatccgaaaattcaatttcaagcaaactgttctcaagttttcgagggatatagtaatagcagagcaaaaaaacactggaaaaagcttgtacctattgaaacaaaggataattaaaaatccaaaatgtatcaaaagcctagacaattttaatcactattgcctcttaacaatttgcgcactatgacaatcatgctctcataatgtaacaaaagacacattaggatactagtactgacactgacaccaaggttacatagtcccacatcgaaggagtttaggtagagacatcggtttatataactaagcgtgac

SEQ ID NO: 168 - DNA, 434 bp, Glycine max
  source 1..434: mol_type = genomic DNA
ttgccaacaagatataacaagaagcctaatatttcaaaaggccttttggtccttaaaaactaaggaatgcatctccagaaagaagtacaatcataatagttgctaagaatgcagcaaaattgataaattaatcgagaaaccacaccacaaatcacacatggccattagagaaaaagaaaggtttcaggaaaataaaagaaaagaaaaagtcaattactgcttagctacctctctatattcccatgtgccccttgcttgaaattggaaccactaccaggcaacagagttgttccctttcaagcaataagagttagaaatatttttatatcaaccgaagtggcaaaaagtcagaaaccatgacatacgtttactttgttagtcccacattggatagttttagtaaaacacaggtcattatatagctaaacgctaac

SEQ ID NO: 169 - DNA, 436 bp, Glycine max
  source 1..436: mol_type = genomic DNA
aatatttcaaagcacacttaggtccttaaaaatgcaatgttacaattccaaaaggaagtataatcatagtagaagccaagaatgcagaaaaattaacaaagtgatcgaggaatcattgccacaaatcactcatggctattagagaaaaaaaaaagtttcaagaaaagaaaagaaaaagttaattactacttagctacttctttgtaaactcaggtgccccttgcatgcaattgataccacaccaggcaccagagctgttccttttcatgcaataagagtaagagacatttttttatgaactcagatggcgaagtgtttgaaaccatgacaggcgttactctggtagtccgacatagagagttttaataaaacacaaatgtagtcccacattgaacagttttaataaaacacagtctttatataactaaacgctgag

SEQ ID NO: 170 - DNA, 428 bp, Glycine max
  source 1..428: mol_type = genomic DNA
cttaggtggctcttgcttgaaatgggtactgtactggaattagagtttatcttttcatgcacaaagttttggccctcgttctactacagtatacagcagtatggatatggttcacattaattctccgaaagtaatctatccctgcgggctagcctgagaaagggtgtttataatcttgtagtgatagaatcaagcatcataagagctttcaaagcctctaatgagtatttctttttctagaaaagatgaatatttgggatcaatgctgctgctgttatgtaggaaacaccgtggcaggaagattgtatttgtatccttgtgcaatactcagaccaattctgttagaaaaatgacacaaaggccgtgatgctagtcccacatcgagtgtttttaacaaaacaggcgcatatatattactggacgctgag

SEQ ID NO: 171 - DNA, 432 bp, Glycine max
  source 1..432: mol_type = genomic DNA
catgctgtgtagtgaaagacatagacagtcaaaaggcctgagaagggtatatattgtaaagatgatgatttttcttacagatggtttataccgctttatgcttcttcagcgtagaacttatcaaagagaacactatccatagctgaatcaagttggttcaggcttttgttataatcacagtctaggagtctaggacacttttattttgcattcattttcggattgttgacgcctttgtataaattttacaatctctcagtaaaagatcgaacacttctgataatgttttaggaatttaactctcattctataagctttgaccaagtctgaaccctagacaattgcctctttaacaacttgacacagaaataataatagtcccacatcgagagcgtttagctacaaacatgtgtttatataattaagcataac

SEQ ID NO: 172 - DNA, 434 bp, Glycine max
  source 1..434: mol_type = genomic DNA
gtttatgtttggcattctttaatctttagtatcagaataattgatcacattttgaaatgccaatagaaagacactccttaaacaaaagctatcgtgtatgaaaagctgcagagaaaaatcaaatgtacaagattttaaataaagacatgctaaactgtccaatatgtaactagttgaacaggctgcatttctctgtgtttttgcttccgcagggaggaatacaaacatctaacaacttataccaaagcatatagtacaaattacaatcttttgttttccccaaattaatccaatttatctttcatttcatccagaaagaagctctttgagattgttagtgttaaactgcaaactttcttcttacttatgtcccacataggaaagaaaaggagacaacaaaatctttatatgccctctgacaaagttgtctgaat

SEQ ID NO: 173 - DNA, 417 bp, Glycine max
  source 1..417: mol_type = genomic DNA
catttaaaataatgaatgactaatccattaactaataactatagttgattactttttagttaattaggatgttaggaatacctgaatccaccgacttaactattatcataatcagttcattcaatttgaatttgtaggatgcacctctatgttttcctcttcactttctagtggacacaagaccctgtgtcacacacagttaatagagaaagatcatttacatgtaagcggaacccattgcattctacagcaatattttggggttggtgttataatacaagacggataaaatagctagagagaatccaatcaaatcccacattccaaattagactatccattgtaggccaagtcccacattggttagaatattaggcaacgaaacctttataaatcttctgacaagcacttcagcat

SEQ ID NO: 174 - DNA, 439 bp, Glycine max
  source 1..439: mol_type = genomic DNA
ggcatgtgactttttatttaaattattctcagaaatattaaaatataatcaaatatatttttttataagatactggaaataacatttttaggaatgtaacgaaagccccattacaaacacttgaactcagggatcgatccaacttaattattatctgctacttcaaattcaaatttataggccctacctttatgttttgctctgcactttcttaaatggaaacaagtaacacaatagagtaagatcatttacttgtaagcggaaactgttgcatcaactgcaatattttcgggttagttttataatataacaatgaagagaattgccgagaagatcatatcaagtcccatattccatttattggcctttacaagtcccacatcggtccaaatattagacaaagaaacatttatatatcgtctgacaaacctgtaagatt

SEQ ID NO: 175 - DNA, 169 bp, Zea mays
  source 1..169: mol_type = genomic DNA
ggattgtgcgggcaaagtattttcgtcgaaagagaagcaacgaaaaagcccactgctccgagacaccgttccacgaaacctgtccgcactcatctcatcgtttagcaccacctcgccagtccgcagtcctctatacctgcttaaatattcgtccagaccgcccaccctc

SEQ ID NO: 176 - DNA, 200 bp, Zea mays
  source 1..200: mol_type = genomic DNA
ctgttttcatccggcctgtgatggaaattgtgctgcaaagtgcgcagggccgttggcccgacgacaagtatttttcgtcgagagaagcaacgaaagcccactcctccaagccacccttcgctcaagtcatatttagcaccacctcggcagtcgacactgcagcacaccaacttaaatattcatcccgatctccctcactc

SEQ ID NO: 177 - DNA, 200 bp, Zea mays
  source 1..200: mol_type = genomic DNA
aaactgtgcttgtgcagggccgttgggccacgtgcccacgtcaaagtattctagtcgaaagagaagcaacgaaagcccactactactcctaggcaccgttgccacgagacctgtccgcactcatctcatcgtttagcaccaatgcgccagtccgcagtccgctgtaccagcttaaatattcgtccctaccgcgctccatc

SEQ ID NO: 178 - DNA, 200 bp, Zea mays
  source 1..200: mol_type = genomic DNA
catatagggatcagctaagcttatcataccttcctttttttttctcattcagccactactgcagcctacaaaggatatcataatgggccgacccagtcacccaggacgcaatatgttggtcaatcccaccagttagtaccacctcggtagctcagatgagtagaagataccttaaaagttcagctcaggcaacttgcagc

SEQ ID NO: 179 - DNA, 200 bp, Zea mays
  source 1..200: mol_type = genomic DNA
catggtccataggtatcaagatccagcattgcacttgggccaaagattgagctctgcgaagtgtcaactacaatcatctagatgggctcgacctgaaaggccacacgatttgccagaactcccaccaattaattagtaccacctcggttgctcgatttagtagaagccagcttaaaagttcagctctggggtccggtagc

SEQ ID NO: 180 - DNA, 181 bp, Zea mays
  source 1..181: mol_type = genomic DNA
catccaggtgaggcatcaagctactactgcctcgattggctggacccgaagcccacatgtaggataccagaatgggccgacccaggacgcagtatgttggccagtcccaccggttagtgccatctcggttgctcacatgcgtagaagccagcttaaaaatttagctttggtaactcacagc

SEQ ID NO: 181 - DNA, 200 bp, Zea mays
  source 1..200: mol_type = genomic DNA
catcaataagataaagctggtgcaggctctcgacctgaagcccacacactggagagcaacgatataccagaatggagcggcccagtacacgatctgctgggattgctggccagtggccagtcccgcagccgattagcaccacctcggtggcacagacgaacgaacgctaatttaaaagcttagctctggagcttggcacc

SEQ ID NO: 182 - DNA, 200 bp, Zea mays
  source 1..200: mol_type = genomic DNA
gcgaggcatcaacaagataaagctggtgcaggctctcgacctgaagcccacacacaggagagcaacaatataccaggaatggagcggcccagtacgcgatctgctgggattgctggctagtcccgcacccgattagcaccacctcgcttgctcatacgagcgaacgcaaatttaaaaggttagctctggagtttggcagc

SEQ ID NO: 183 - DNA, 200 bp, Zea mays
  source 1..200: mol_type = genomic DNA
cctaactaataagtatcggaggcaacaacgcccgctgggctgcggcccattaacatagcactagacggacaccgccgcagtcagcgttcagccggatgcagtgcgatcggcttcatccgtttagtcccacctcgcccagccaaagcagcggggaggcccggactcgctacaaaaggagacggaggttggctgtttagagc

SEQ ID NO: 184 - DNA, 200 bp, Zea mays
  source 1..200: mol_type = genomic DNA
gccagctgcggtggaaacgactgcggaccgggctagcatcgacgtattgccgtattagtagcccagaaagcccacgcaaccagatagaggtctgaatgcctgtactttgtggtgcttcagtttagtcccacctcggtggctttcagcgggagagagcagagaaggcttataaaagcagacctcagtcaacataacaattc

SEQ ID NO: 185 - DNA, 200 bp, Zea mays
  source 1..200: mol_type = genomic DNA
tcaataaacaattggagcgagagagactctgctgaccgggccatcattgacgtattggtagcccagaaagcccacacagtcatacagaggtctgaatgtctgtgctttatggtgcttgagtttagtcccacctcggtggctttcagcgggagagagcagagaaggcttataaaagcagacctcagtcaacataacaattc

SEQ ID NO: 186 - DNA, 200 bp, Zea mays
  source 1..200: mol_type = genomic DNA
gaccccatctatgccgtaattctggcttcacccctgatcaagctatcgtaccttaaatgcgtcgtttcacgtttttcaccttgttggaggtctgaatgtctgtgctttgtggtgcttcaatttagtcccaccttgtcggctttcaacgggagggagcggagaaggcttataaaagcagaccctagtcaacataacaattc

SEQ ID NO: 187 - DNA, 200 bp, Zea mays
  source 1..200: mol_type = genomic DNA
ggtggaaacggactcgagcgagagagactctgcggaccgggccagcatcgatgtattggtagcccagaaagcccacgcaaccagacagaggtatgaatgttgtgctttgtggtgcttcagtttagtcccacctcggcggttttcagcggaagggagcagagaaggcttataaaagcagacctcagtcaacataacaattc

SEQ ID NO: 188 - DNA, 200 bp, Zea mays
  source 1..200: mol_type = genomic DNA
tccctttgtcgccttgctgttcgctccgtttgttgggccggacatgtggctttctgggccgcgcacgtagagtcctcctggccagtgtcgggctctggctggctgcctgctcttttttctgtagtcccaaaccgcttgctgaggctaatatcagccgggaacaacgctatttaggattgactgaactctgtgtaagaggc

SEQ ID NO: 189 - DNA, 200 bp, Zea mays
  source 1..200: mol_type = genomic DNA
cctttgtggcttcgcttcattctcttcatccgtttgtgaccggagtcagcggctgtcgaccgcggcgcatcctctgactagcactgcgtctggctggctctggctgcttcagcttgctttagttcccaaacccgcttgctgtagctaataccagcagggagcgctgctatttaggatggactgggcgctgcgagagaagc

SEQ ID NO: 190 - DNA, 200 bp, Zea mays
  source 1..200: mol_type = genomic DNA
ggccttcgcttcctcgctcttcgctcctttgttgggccggacaacaagcggctgtctgggccgcgcacgcagtcctctggtccagtgggcaactgcctctggctgcttgctcttttccttaatcccaaaccgctcgatgaagctaataccagctggggggcgctgctatttaggagagactaagcgccacccgtagatgc

SEQ ID NO: 191 - DNA, 200 bp, Zea mays
  source 1..200: mol_type = genomic DNA
ttcgcttcatttctcttcactccgtttgttgggccggacatcaagcggctgtctgggccgcgcacgcagtcctcttgactagcactgcgtctggctggctctggctgcttcagctttgctttagttccaaaccgcttgctgtagctaataccagcagggagcgctgctatttaggatggactgggcgctgcgagagaagc

SEQ ID NO: 192 - DNA, 200 bp, Zea mays
  source 1..200: mol_type = genomic DNA
tcaataaacaattggagcaagagagactctgctgaccgggccatcatcgacgtattggtagcccagaaagcccacgcagtcatacagaggtctgaatgtctgtgctttgtggtgcttgagtttagtcccacctcgacggctttcagcgggagagagcagagaaggcttataaaagcagacctcagtcaacataacaattc

SEQ ID NO: 193 - DNA, 200 bp, Zea mays
  source 1..200: mol_type = genomic DNA
gactcgagaacttagagctagagagactctgcggaccgggccagcatcgacgtattggtagcccagaaagcccacgcaaccagacagaggtctgaatgcatgtgctttgtggtgcttcagtttagtcccacctcggcggctttcagcgggagagagcagagaaggcttataaaagcagacctcagttaacataacaattc

SEQ ID NO: 194 - DNA, 200 bp, Zea mays
  source 1..200: mol_type = genomic DNA
ggccccttctatgtcgtaaatctaggtccacccccaatcaagcaatcctaccttaaatacatcgtttcacgtttttgagcttgttggaggtgtgaaagtttgtgctttgtggtgcttcagtttagtcccacctcggtggctttcagtgagagggagcgaagaaggcttataaaagcacaccccaatcaacataacaattc

SEQ ID NO: 195 - DNA, 200 bp, Zea mays
  source 1..200: mol_type = genomic DNA
tcaatatacaattggagcgagagagactctgctgaccgggccagcatcgacgtattggtagcccagaaagcccacgcagtcatacagaggtctgaatgtatgtgctttgtggtgcttgagtttagtcccacctcggaggctttcagcgggagagagcaaagaaggcttataaaagcagacctcagtcagcataacaattc

SEQ ID NO: 196 - DNA, 200 bp, Zea mays
  source 1..200: mol_type = genomic DNA
gccagctgcggtggaaacgactgcggaccgagccagcatcgacgtattgccgtattggtagcccagaaagcccacgcaaccggacagaggtctgaatgcctgtactttgtggtgcttcagtttagtcccacctcggtggctttcagcgggagagagcagagaaggcttataaaagcagacctcagtcaatataacaattc

SEQ ID NO: 197 - DNA, 200 bp, Zea mays
  source 1..200: mol_type = genomic DNA
gagtccagaacttggagcgagagagactctgctgaccgggccagcatggacgtattggtagcccagaaagcccacgcaaccagatagaggtcttaatgccctttctttgtggtgctttagtttagtcccacctcgacggctttcagcgggagggagcagagaaggcttataaaagaagatctcaatcaaattaacaattc

SEQ ID NO: 198 - DNA, 200 bp, Zea mays
  source 1..200: mol_type = genomic DNA
gactcgagaacttggagcgagagagactctgctgaccgggccagcatggacgtattggtagcccagaaagcccacgctaccagacagaggtctgaatgactgttctttgtggtgcttcagtttagtcccacctcgacggctttcagcgggaggaagcagagaaggcttataaaagcagatcctagtcaacataacaattc

SEQ ID NO: 199 - DNA, 192 bp, Zea mays
  source 1..192: mol_type = genomic DNA
aacggactcgagcgagagagactctgcggaccgggccagcatcgatgtatttggtagcccagagcccacgcaaccagacagaggtatgaatgttgtgctttgtggtgcttcagtttagtcccacctcggcggttttcagcggaagggagcagagaaggcttataaaagcagacctcagtcaacataacaatt

SEQ ID NO: 200 - DNA, 199 bp, Zea mays
  source 1..199: mol_type = genomic DNA
ggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaatt

SEQ ID NO: 201 - DNA, 418 bp, Zea mays
  source 1..418: mol_type = genomic DNA
aggaaaagaagaggtgattactgtacctattcgtctttgtatcggaatataaatttatcactattttatgataaagtaaatctgtttccctgtagagttaattaattaatgtaagtataagcgtaatttatagggcactagtaggactgtcgactgtgcgctcggcccggataatgcgtcaaaagcgaagacgtgcacgtgggatgggaaaacacgaagcgtggtctgctttttcgcatgatatctgggccgcaccaaagaatccagcccacgcggcgtggcgccgtcgttacggcttgcgggggaaggaaacgagggacgaaccgagatttagcaccagaccggccagcgagcattgcagacaccggcttataagttcagctgcgactaccactccgggaggtccgagttccactcg

SEQ ID NO: 202 - DNA, 625 bp, synthetic construct
  source 1..625: mol_type = other DNA; note = Recombinant
ttctatttgtgctacatatattagacaaggaaaataacatatgttattttgaaatcacgtatatttactataaattacaatgattaacaacttaaaatatttaaatgaaaatcatattaatgactctctaaattttatctgtgtcacataaatgaaaaacaaaaaataacaaatattgtattcgcacgggcgcatgtgtctagttagttataaacgaagaaataaggggctgatttcgaaataaacgttcttagaattggaagaaatgttcagtttctaaacttgtaggactaaagcaataacttttatttaatttattttcttttatgtttctcccacatcgatcatacatataactatacagcagtataagaactctagcgaagcaataatgctccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 203 - DNA, 474 bp, synthetic construct
  source 1..474: mol_type = other DNA; note = Recombinant
ggaaacttctgtttgtccccatactccaaaaacaaaaccattttttttttatcttcgtttttgtttgctttgactgtgagttgaggcccaactttctgcttctgtccgactctatttgatgaattttgtttgcctcctgtgatgtgaaggatgtatcattgaaagggaacgtgtctcaatgatcccacatcggccaaatatgctcattacattgcgtttatatagtcccaggaaaacatatggattcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 204 - DNA, 721 bp, synthetic construct
  source 1..721: mol_type = other DNA; note = Recombinant
tctacaaaacattataaaaagtaagatataacaactttttttttaaaaaaatcaataggaaatattatcggttcagttaatttacagagagatatttatttcatatgtgccagaagtatttcagttccttatgaaaaatcagaaaaatgtatggaataaaatataataatcgatactaataatagaacaaaataaatggtaaaatgtcaaatcaaaactaggctgcagtatgcagagcagagtcatgatgatactacttactacaccgattcttgtgtgcagaaaaatatgttaaaataattgaatctttctctagccaaatttgacaacaatgtacaccgttcatattgagagacgatgcttcttgtttgctttcggtggaagctgcatatactcaacattactccttcagcgagttttccaactgagtcccacattgcccagacctaacacggtattcttgtttataatgaaatgtgccaccacatggattcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 205 - DNA, 787 bp, synthetic construct
  source 1..787: mol_type = other DNA; note = Recombinant
aggtggagttgttccagatttagttttcgacttagatgatgcatggaactggctagtgacgtggatggtggtaggttactttcaggtcatgattttttgtttctaaatgatactcacactcccttccagttttttttttttaaactcagctcccttgcttcctccaccggttatcataatactgaaccaaatcaaacattacagtcaaggtactatgaatatgaaacctgaaatcctatgaatgtcataaatttattttaaataataaatttatttagaataatatttttttgggtaagagttataaaataaaatacaaaaaaaaaacctaatatcaatttttcactgactccgtttatattgagacttgagaaagatggttcccgtttgctcccggtggaggctccgaggctgtgtatatactcgacattactttagcttgttttgttgtttctttccctttcccacaagactcaggtctcgttcgcaaacgagtcccacaccgtctaaacttaccacaatattagcgtttataattagatgcactgcatcacttattcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 206 - DNA, 824 bp, synthetic construct
  source 1..824: mol_type = other DNA; note = Recombinant
aaattacacggtagtacatcctattatagatcgatgaattcttaactactcgtgtcccttaggccaggcctgttttcttgcacaatttctgaaatgtatacggtttccacttcaacctttttaaccgcacaaagttttaaccagatttatataatttatttttgaatccccaatacatatcattataacatatcaattatcaaatatttcaataacctcatgatatggcaatgaatacatcttcttctcaatgaacagagatttctgaaaaagattaggaaagtgaaagcatactcgtttgcaatgtaaaactgatacttccccaaaatcatcatattccaaatatgccctggtgttactgaccaaaaccagaaaaaagaaacggaagacatatacgtctaaacggagaaatttcaaaaaacaaaaattggatcatttctcgatttgtgggtgtcatcttgtgcagggcatgctaatcttctctttaccctttcccacaagactcagcgcatgttgtctcgtctcatccaagtcccacaccgcctaaacttaacacaatattagtatttataatgacatacaacattcaagatgttcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 207 - DNA, 965 bp, synthetic construct
  source 1..965: mol_type = other DNA; note = Recombinant
aattatttaatcctaaaaatcaatcttacaaggaaccactataaaactagttgaacccatcgaaaactaactacagttgacaaaatctatttggttggttgattttttttaattaaaaacatccaatcttaatgatataattatagcttaatattataagatttttataaaaattatatttattttgtttctaattatgctaagagatattattatttgttatttaacttaaatattatcacaaacttgattgaaacttatgtttaatttaaaatattatatgtcatgagttatgactccaataatcacaattataaagtgaagtttaatttttagtattacaaatatttttttgttgtttaattttaattacttaatgtattatgttaataattaaaaatacaaattatttattattaatgcaatcacagtttgtggatttgacaaaagaaataggggatctaaaattgtagataagccaaagttaaaacttgaattgactattttttgctctttactctgcaccaactttactattccttcttttagtgtgagcttcatgcatcttgttcaccgcaattccgctcggtgaaagttgcacaattcactcacaatctgtttctggtctgttaggtttgttacttggagtgacacgatgacgcaacagtacaagtcccacatcgtttgagtatacagttttcaagcagtttatattcccatagccttagcaagagcttcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 208 - DNA, 712 bp, synthetic construct
  source 1..712: mol_type = other DNA; note = Recombinant
ttttttaataaaaaaaattcaatgggagatactatggattcaattaccttactgattttatttcatatgtgccagaagtatttcagtttattttgaaaaatcagaaaaaaaatgtctggaataaaatataataagcgatactaataaataattgaacaagataaatggtaaaatgtcaaatcaaaactaggctacagagtgcagagcagagtcatgatgaatgacagctagttctacttactacaccgattcttgtgtacataaaaatattttaaaataattgaatctttctttagccagctttgacaacaatgtacaccgttcgtacttcttactggtaggcaatgcttcttgtttgctttcggtggaaggtgtatatactcaacattacttctttttcagcgtgttttcttacgggagtcccacaccgcccaaaactaatacagtattcttgtttataaagaagtgcaccacttcaattgttcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 209 - DNA, 655 bp, synthetic construct
  source 1..655: mol_type = other DNA; note = Recombinant
cgataaaaatgttttaaacgatatatattataaaaaaaaacgtttcaaaaataaatacaaaaatgtttttaaatatatataatttaactcattaaagaaaataaaaatgcaagtgcggtgacaagacaagctaaaagttgcaaaagaaatggcagggctataaggctcacctactcctggatttaccaaattttggttcgtccctatactcgaaaaataaaacaaaataaatttcagtatcttcgtttttgtatgctttgactgtgaggcgaggccaactttcttcttctgtctgagatgaattttgtttgcctcctgtgaaggatgtatcattcaaagtgaatgttttgcaactgccagtagtcccacatcgaccaaatattcttattacagtgtgtttatatagcacctggagaaggaatgggttcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 210 - DNA, 628 bp, synthetic construct
  source 1..628: mol_type = other DNA; note = Recombinant
gaaaagcattcagaatatttgagcctctaaaaacttttcttctttttctttgaggagtgtaatgtggatcccaagtaaccaagaaaagcaatccgaaaattcaatttcaagcaaactgttctcaagttttcgagggatatagtaatagcagagcaaaaaaacactggaaaaagcttgtacctattgaaacaaaggataattaaaaatccaaaatgtatcaaaagcctagacaattttaatcactattgcctcttaacaatttgcgcactatgacaatcatgctctcataatgtaacaaaagacacattaggatactagtactgacactgacaccaaggttacatagtcccacatcgaaggagtttaggtagagacatcggtttatataactaagcgtgaccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 211 - DNA, 662 bp, synthetic construct
  source 1..662: mol_type = other DNA; note = Recombinant
ttgccaacaagatataacaagaagcctaatatttcaaaaggccttttggtccttaaaaactaaggaatgcatctccagaaagaagtacaatcataatagttgctaagaatgcagcaaaattgataaattaatcgagaaaccacaccacaaatcacacatggccattagagaaaaagaaaggtttcaggaaaataaaagaaaagaaaaagtcaattactgcttagctacctctctatattcccatgtgccccttgcttgaaattggaaccactaccaggcaacagagttgttccctttcaagcaataagagttagaaatatttttatatcaaccgaagtggcaaaaagtcagaaaccatgacatacgtttactttgttagtcccacattggatagttttagtaaaacacaggtcattatatagctaaacgctaaccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 212 - DNA, 664 bp, synthetic construct
  source 1..664: mol_type = other DNA; note = Recombinant
aatatttcaaagcacacttaggtccttaaaaatgcaatgttacaattccaaaaggaagtataatcatagtagaagccaagaatgcagaaaaattaacaaagtgatcgaggaatcattgccacaaatcactcatggctattagagaaaaaaaaaagtttcaagaaaagaaaagaaaaagttaattactacttagctacttctttgtaaactcaggtgccccttgcatgcaattgataccacaccaggcaccagagctgttccttttcatgcaataagagtaagagacatttttttatgaactcagatggcgaagtgtttgaaaccatgacaggcgttactctggtagtccgacatagagagttttaataaaacacaaatgtagtcccacattgaacagttttaataaaacacagtctttatataactaaacgctgagcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 213 - DNA, 656 bp, synthetic construct
  source 1..656: mol_type = other DNA; note = Recombinant
cttaggtggctcttgcttgaaatgggtactgtactggaattagagtttatcttttcatgcacaaagttttggccctcgttctactacagtatacagcagtatggatatggttcacattaattctccgaaagtaatctatccctgcgggctagcctgagaaagggtgtttataatcttgtagtgatagaatcaagcatcataagagctttcaaagcctctaatgagtatttctttttctagaaaagatgaatatttgggatcaatgctgctgctgttatgtaggaaacaccgtggcaggaagattgtatttgtatccttgtgcaatactcagaccaattctgttagaaaaatgacacaaaggccgtgatgctagtcccacatcgagtgtttttaacaaaacaggcgcatatatattactggacgctgagcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 214 - DNA, 660 bp, synthetic construct
  source 1..660: mol_type = other DNA; note = Recombinant
catgctgtgtagtgaaagacatagacagtcaaaaggcctgagaagggtatatattgtaaagatgatgatttttcttacagatggtttataccgctttatgcttcttcagcgtagaacttatcaaagagaacactatccatagctgaatcaagttggttcaggcttttgttataatcacagtctaggagtctaggacacttttattttgcattcattttcggattgttgacgcctttgtataaattttacaatctctcagtaaaagatcgaacacttctgataatgttttaggaatttaactctcattctataagctttgaccaagtctgaaccctagacaattgcctctttaacaacttgacacagaaataataatagtcccacatcgagagcgtttagctacaaacatgtgtttatataattaagcataaccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 215 - DNA, 662 bp, synthetic construct
  source 1..662: mol_type = other DNA; note = Recombinant
gtttatgtttggcattctttaatctttagtatcagaataattgatcacattttgaaatgccaatagaaagacactccttaaacaaaagctatcgtgtatgaaaagctgcagagaaaaatcaaatgtacaagattttaaataaagacatgctaaactgtccaatatgtaactagttgaacaggctgcatttctctgtgtttttgcttccgcagggaggaatacaaacatctaacaacttataccaaagcatatagtacaaattacaatcttttgttttccccaaattaatccaatttatctttcatttcatccagaaagaagctctttgagattgttagtgttaaactgcaaactttcttcttacttatgtcccacataggaaagaaaaggagacaacaaaatctttatatgccctctgacaaagttgtctgaatcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 216 - DNA, 645 bp, synthetic construct
  source 1..645: mol_type = other DNA; note = Recombinant
catttaaaataatgaatgactaatccattaactaataactatagttgattactttttagttaattaggatgttaggaatacctgaatccaccgacttaactattatcataatcagttcattcaatttgaatttgtaggatgcacctctatgttttcctcttcactttctagtggacacaagaccctgtgtcacacacagttaatagagaaagatcatttacatgtaagcggaacccattgcattctacagcaatattttggggttggtgttataatacaagacggataaaatagctagagagaatccaatcaaatcccacattccaaattagactatccattgtaggccaagtcccacattggttagaatattaggcaacgaaacctttataaatcttctgacaagcacttcagcatcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 217 - DNA, 667 bp, synthetic construct
  source 1..667: mol_type = other DNA; note = Recombinant
ggcatgtgactttttatttaaattattctcagaaatattaaaatataatcaaatatatttttttataagatactggaaataacatttttaggaatgtaacgaaagccccattacaaacacttgaactcagggatcgatccaacttaattattatctgctacttcaaattcaaatttataggccctacctttatgttttgctctgcactttcttaaatggaaacaagtaacacaatagagtaagatcatttacttgtaagcggaaactgttgcatcaactgcaatattttcgggttagttttataatataacaatgaagagaattgccgagaagatcatatcaagtcccatattccatttattggcctttacaagtcccacatcggtccaaatattagacaaagaaacatttatatatcgtctgacaaacctgtaagattcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 218 - DNA, 398 bp, synthetic construct
  source 1..398: mol_type = other DNA; note = Recombinant
ggattgtgcgggcaaagtattttcgtcgaaagagaagcaacgaaaaagcccactgctccgagacaccgttccacgaaacctgtccgcactcatctcatcgtttagcaccacctcgccagtccgcagtcctctatacctgcttaaatattcgtccagaccgcccaccctccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtctttttttt

SEQ ID NO: 219 - DNA, 429 bp, synthetic construct
  source 1..429: mol_type = other DNA; note = Recombinant
ctgttttcatccggcctgtgatggaaattgtgctgcaaagtgcgcagggccgttggcccgacgacaagtatttttcgtcgagagaagcaacgaaagcccactcctccaagccacccttcgctcaagtcatatttagcaccacctcggcagtcgacactgcagcacaccaacttaaatattcatcccgatctccctcactccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtctttttttt

SEQ ID NO: 220 - DNA, 429 bp, synthetic construct
  source 1..429: mol_type = other DNA; note = Recombinant
aaactgtgcttgtgcagggccgttgggccacgtgcccacgtcaaagtattctagtcgaaagagaagcaacgaaagcccactactactcctaggcaccgttgccacgagacctgtccgcactcatctcatcgtttagcaccaatgcgccagtccgcagtccgctgtaccagcttaaatattcgtccctaccgcgctccatccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtctttttttt

SEQ ID NO: 221 - DNA, 429 bp, synthetic construct
  source 1..429: mol_type = other DNA; note = Recombinant
catatagggatcagctaagcttatcataccttcctttttttttctcattcagccactactgcagcctacaaaggatatcataatgggccgacccagtcacccaggacgcaatatgttggtcaatcccaccagttagtaccacctcggtagctcagatgagtagaagataccttaaaagttcagctcaggcaacttgcagccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtctttttttt

SEQ ID NO: 222 - DNA, 429 bp, synthetic construct
  source 1..429: mol_type = other DNA; note = Recombinant
catggtccataggtatcaagatccagcattgcacttgggccaaagattgagctctgcgaagtgtcaactacaatcatctagatgggctcgacctgaaaggccacacgatttgccagaactcccaccaattaattagtaccacctcggttgctcgatttagtagaagccagcttaaaagttcagctctggggtccggtagccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtctttttttt

SEQ ID NO: 223 - DNA, 410 bp, synthetic construct
  source 1..410: mol_type = other DNA; note = Recombinant
catccaggtgaggcatcaagctactactgcctcgattggctggacccgaagcccacatgtaggataccagaatgggccgacccaggacgcagtatgttggccagtcccaccggttagtgccatctcggttgctcacatgcgtagaagccagcttaaaaatttagctttggtaactcacagccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtctttttttt

SEQ ID NO: 224 - DNA, 429 bp, synthetic construct
  source 1..429: mol_type = other DNA; note = Recombinant
catcaataagataaagctggtgcaggctctcgacctgaagcccacacactggagagcaacgatataccagaatggagcggcccagtacacgatctgctgggattgctggccagtggccagtcccgcagccgattagcaccacctcggtggcacagacgaacgaacgctaatttaaaagcttagctctggagcttggcacccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtctttttttt

SEQ ID NO: 225 - DNA, 429 bp, synthetic construct
  source 1..429: mol_type = other DNA; note = Recombinant
gcgaggcatcaacaagataaagctggtgcaggctctcgacctgaagcccacacacaggagagcaacaatataccaggaatggagcggcccagtacgcgatctgctgggattgctggctagtcccgcacccgattagcaccacctcgcttgctcatacgagcgaacgcaaatttaaaaggttagctctggagtttggcagccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtctttttttt

SEQ ID NO: 226 - DNA, 442 bp, synthetic construct
  source 1..442: mol_type = other DNA; note = Recombinant
cctaactaataagtatcggaggcaacaacgcccgctgggctgcggcccattaacatagcactagacggacaccgccgcagtcagcgttcagccggatgcagtgcgatcggcttcatccgtttagtcccacctcgcccagccaaagcagcggggaggcccggactcgctacaaaaggagacggaggttggctgtttagagccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcacaattcaaaacaagttttat

SEQ ID NO: 227 - DNA, 442 bp, synthetic construct
  source 1..442: mol_type = other DNA; note = Recombinant
gccagctgcggtggaaacgactgcggaccgggctagcatcgacgtattgccgtattagtagcccagaaagcccacgcaaccagatagaggtctgaatgcctgtactttgtggtgcttcagtttagtcccacctcggtggctttcagcgggagagagcagagaaggcttataaaagcagacctcagtcaacataacaattccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcacaattcaaaacaagttttat

SEQ ID NO: 228 - DNA, 442 bp, synthetic construct
  source 1..442: mol_type = other DNA; note = Recombinant
tcaataaacaattggagcgagagagactctgctgaccgggccatcattgacgtattggtagcccagaaagcccacacagtcatacagaggtctgaatgtctgtgctttatggtgcttgagtttagtcccacctcggtggctttcagcgggagagagcagagaaggcttataaaagcagacctcagtcaacataacaattccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcacaattcaaaacaagttttat

SEQ ID NO: 229 - DNA, 442 bp, synthetic construct
  source 1..442: mol_type = other DNA; note = Recombinant
gaccccatctatgccgtaattctggcttcacccctgatcaagctatcgtaccttaaatgcgtcgtttcacgtttttcaccttgttggaggtctgaatgtctgtgctttgtggtgcttcaatttagtcccaccttgtcggctttcaacgggagggagcggagaaggcttataaaagcagaccctagtcaacataacaattccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcacaattcaaaacaagttttat

SEQ ID NO: 230 - DNA, 442 bp, synthetic construct
  source 1..442: mol_type = other DNA; note = Recombinant
ggtggaaacggactcgagcgagagagactctgcggaccgggccagcatcgatgtattggtagcccagaaagcccacgcaaccagacagaggtatgaatgttgtgctttgtggtgcttcagtttagtcccacctcggcggttttcagcggaagggagcagagaaggcttataaaagcagacctcagtcaacataacaattccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcacaattcaaaacaagttttat

SEQ ID NO: 231 - DNA, 442 bp, synthetic construct
  source 1..442: mol_type = other DNA; note = Recombinant
tccctttgtcgccttgctgttcgctccgtttgttgggccggacatgtggctttctgggccgcgcacgtagagtcctcctggccagtgtcgggctctggctggctgcctgctcttttttctgtagtcccaaaccgcttgctgaggctaatatcagccgggaacaacgctatttaggattgactgaactctgtgtaagaggccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcacaattcaaaacaagttttat

SEQ ID NO: 232 - DNA, 442 bp, synthetic construct
  source 1..442: mol_type = other DNA; note = Recombinant
cctttgtggcttcgcttcattctcttcatccgtttgtgaccggagtcagcggctgtcgaccgcggcgcatcctctgactagcactgcgtctggctggctctggctgcttcagcttgctttagttcccaaacccgcttgctgtagctaataccagcagggagcgctgctatttaggatggactgggcgctgcgagagaagccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcacaattcaaaacaagttttat

SEQ ID NO: 233 - DNA, 442 bp, synthetic construct
  source 1..442: mol_type = other DNA; note = Recombinant
ggccttcgcttcctcgctcttcgctcctttgttgggccggacaacaagcggctgtctgggccgcgcacgcagtcctctggtccagtgggcaactgcctctggctgcttgctcttttccttaatcccaaaccgctcgatgaagctaataccagctggggggcgctgctatttaggagagactaagcgccacccgtagatgccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcacaattcaaaacaagttttat

SEQ ID NO: 234 - DNA, 442 bp, synthetic construct
  source 1..442: mol_type = other DNA; note = Recombinant
ttcgcttcatttctcttcactccgtttgttgggccggacatcaagcggctgtctgggccgcgcacgcagtcctcttgactagcactgcgtctggctggctctggctgcttcagctttgctttagttccaaaccgcttgctgtagctaataccagcagggagcgctgctatttaggatggactgggcgctgcgagagaagccaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcacaattcaaaacaagttttat

SEQ ID NO: 235 - DNA, 427 bp, synthetic construct
  source 1..427: mol_type = other DNA; note = Recombinant
ggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 236 - DNA, 428 bp, synthetic construct
  source 1..428: mol_type = other DNA; note = Recombinant
gcgtggtctgctttttcgcatgatatctgggccgcaccaaagaatccagcccacgcggcgtggcgccgtcgttacggcttgcgggggaaggaaacgagggacgaaccgagatttagcaccagaccggccagcgagcattgcagacaccggcttataagttcagctgcgactaccactccgggaggtccgagttccactcgcaagctgactctagcagatctccatgacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcttttttt

SEQ ID NO: 237 - DNA, 21 bp, synthetic construct
  source 1..21: mol_type = other DNA; note = Recombinant
acaattcaaaacaagttttat

SEQ ID NO: 238 - DNA, 4622 bp, synthetic construct
  source 1..4622: mol_type = other DNA; note = Recombinant
atgggatctaagaagagaagaattaaacaagatatgagtgacctggtgctagggttggctataggcattggctccgtgggggttggcattcttaataaggtgaccggcgaaataattcataaaaactcacgcatctttccagcagcccaggctgagaacaatctggtccgtagaaccaaccggcagggtcgaaggttagccaggcgcaagaagcacagacgggtccggctcaacaggcttttcgaggagtctggtttgatcaccgatttcactaagatttctatcaacctgaatccttatcagctgcgcgttaaaggtctcacagacgaacttagcaacgaagagttgttcatcgccctgaaaaatatggtcaagcatcgcggcattagctacctggacgacgcttcggatgatggcaacagtagtgtaggtgactacgctcagatcgtgaaagagaactcgaagcaattggagaccaagaccccgggccaaattcaactcgaaaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtaccagacgtatggacagttacgaggcgattttaccgttgaaaaggatggtaagaagcacaggctgattaatgtgtttccgacctcagcttatcgctctgaggcgctgcgtattttgcagacccaacaggaatttaacccgcaaataacggacgagttcataaaccgatacttagagattcttacaggtaaacgtaaatactatcacggcccaggaaatgaaaagtccaggacagattatggtcgatatcgcacttccggagagactctcgacaatatctttggcattcttataggcaaatgtaccttctaccctgacgaatttagagcagcgaaggcttcatatacagcacaagagtttaatcttctcaacgacctcaacaacttgactgtgcctactgaaaccaaaaagcttagcaaggagcaaaaaaatcaaatcattaactatgttaagaatgagaaagctatggggcccgcaaaattgttcaagtacatagctaagttacttagctgtgacgttgctgatattaagggttaccgtattgacaagtctggtaaagctgaaattcacacctttgaggcttataggaagatgaagacccttgagacacttgacattgagcagatggatagggagactttggacaaactggcatacgtcttgacattgaacaccgaaagggaaggcatccaggaagctctggaacatgaatttgcagatggttcgttcagccaaaaacaggttgacgagctggtccaatttagaaaggcaaactcaagcatattcggtaaaggttggcacaacttcagcgttaagctgatgatggaactcattccagaattatatgaaacctctgaggaacagatgacgattctcacaagattgggtaagcagaaaacaaccagctctagcaataagactaaatacattgacgaaaagctcctcaccgaagagatttataacccggtcgtggcaaagagtgtacggcaagccatcaagatcgttaatgccgctatcaaggagtatggtgattttgataatattgtgattgaaatggcacgcgagactaacgaggacgacgagaagaaagctatacagaagattcaaaaggctaataaggacgagaaggacgccgcaatgctaaaggcggccaatcaatataatgggaaggctgaactacctcatagcgtcttccatggacataagcaattagcaactaaaataagattatggcaccagcaaggcgaacggtgtctttatacaggtaaaacgatatctattcacgacctgattaacaactctaaccagtttgaagtggatgctatcttaccactaagtatcaccttcgacgattcacttgctaacaaggtgctcgtttacgccactgcgaaccaagagaaagggcagaggactccataccaggcccttgacagcatggacgacgcctggagttttagggaattaaaagctttcgtacgtgagtcaaagacgctttcaaataaaaaaaaggagtacttgctcactgaagaagacatctcaaaattcgacgtgcgcaaaaaattcattgagcggaacttagtcgacactcggtacgcatcaagagtagtgttgaacgccctccaggagcactttagggcacataagatcgacaccaaggtttcagttgttaggggtcagtttacatcgcagcttagacgccattggggtatagaaaaaacacgtgatacctaccatcaccatgcagttgacgctctcatcattgcagcatcttctcaacttaatttgtggaaaaagcaaaagaacactctggtctcatatagcgaagatcagctgcttgatattgaaaccggcgagctgatttctgacgacgaatacaaagaatctgtgtttaaggcaccatatcaacactttgtagacacgcttaaatctaaagagtttgaggattcgatccttttcagttaccaagtcgactcaaaatttaaccgtaagatctctgatgcaacaatttatgcgacgaggcaggccaaggtaggtaaggataaggctgacgaaacctacgtgctcggaaaaatcaaagatatttacactcaagatggatatgatgcattcatgaagatatataaaaaggacaaatctaaattccttatgtatcgtcatgacccacagacattcgagaaagttattgagcctatcctggagaactatccgaacaagcaaataaatgagaagggcaaagaagttccatgtaatccgttcctaaagtacaaggaggaacacggatatattagaaaatacagcaaaaagggcaacggcccagaaatcaaaagccttaagtactacgatagtaaactaggaaaccacatcgacattacaccaaaagactctaataataaggtcgtactgcaaagcgtttccccatggcgcgccgatgtgtattttaataagacaacagggaagtacgaaatcttggggttaaaatatgcggatctgcaattcgaaaagggaaccggcacatacaaaatttctcaagaaaagtacaacgacataaagaagaaggaaggggtcgattctgattctgaattcaagttcacactctataagaatgatcttctgctcgtcaaggacacagagacaaaggagcagcagttgttcaggttcttgtctagaactatgccaaaacaaaagcactacgttgaactgaagccttacgataagcaaaaattcgaggggggcgaggcgcttataaaggtcctaggaaatgttgcaaactctgggcagtgtaagaagggcctgggcaagagcaacattagcatctataaggttcgaacggatgtgcttgggaaccagcatatcatcaaaaacgagggagataaaccaaagctggacttcggatcttctattgtggcgcagctctcaagaagggacccggcgctagcggctctgactaatgaccatctcgtggctctggcttgcctgggggggcggcctgctctggacgctgtgaagaaggggctcccacacgctccagagttcatccgcagggtgaacaggaggattgctgagcggacaagccacagggtcgctgactacgctcatgtggtccgcgttctggagttcttccagtgccactcgcatccggctcacgccttcgatgaggccatgacccagttcggcatgtctcggcatgggctggtccagctcttcaggcgggttggcgtgactgagttcgaggctcgctacgggaccctgccaccagcgtcccagcgctgggacaggatcctccaggcgagcggcatgaagagggctaagccaagccctacctcggctcagacgccagaccagacatctctccacgcgttcgctgattcactggagagggacctcgatgctccatccccaatgcatgagggcgaccagaccagggcgtccagccgcaagaggtcacggtccgatagggctgtgacggggccatcggctcagcaggctgtcgaggttagggtgcctgagcagagggacgctctccacctgccactctcctggagggtcaagcgccctaggacgaggatctggggcgggctgccagaccctggcacaccgattgccgcggatctcgctgcctcgtctactgttatgtgggagcaggacgctgctccattcgctggcgctgctgacgatttcccagccttcaatgaggaggagctggcttggctgatggagctgctgcctcagtcggggtcggttggcgggacaatcgctgccgacctggcggcttcgtctaccgtcatgtgggagcaggacgccgcgccgttcgctggcgctgccgacgatttccctgcgttcaacgaggaggagctggcgtggctgatggagctgctgccccagagcgggagcgtcggcgggacaatctgaagcagaacacgcgctga

SEQ ID NO: 239 - protein, 1471 aa, synthetic construct
  source 1..1471: note = Recombinant
MGSKKRRIKQDMSDLVLGLAIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDAILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDFGSSIVAQLSRRDPALAALTNDHLVALACLGGRPALDAVKKGLPHAPEFIRRVNRRIAERTSHRVADYAHVVRVLEFFQCHSHPAHAFDEAMTQFGMSRHGLVQLFRRVGVTEFEARYGTLPPASQRWDRILQASGMKRAKPSPTSAQTPDQTSLHAFADSLERDLDAPSPMHEGDQTRASSRKRSRSDRAVTGPSAQQAVEVRVPEQRDALHLPLSWRVKRPRTRIWGGLPDPGTPIAADLAASSTVMWEQDAAPFAGAADDFPAFNEEELAWLMELLPQSGSVGGTIAADLAASSTVMWEQDAAPFAGAADDFPAFNEEELAWLMELLPQSGSVGGTI

SEQ ID NO: 240 - DNA, 27 bp, synthetic construct
  source 1..27: mol_type = other DNA; note = Recombinant
ggaggtccgagttccactcgcaagaat

SEQ ID NO: 241 - DNA, 353 bp, synthetic construct
  source 1..353: mol_type = other DNA; note = Recombinant
ggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgggaggtccgagttccactcggttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 242 - DNA, 350 bp, synthetic construct
  source 1..350: mol_type = other DNA; note = Recombinant
tttcatccggcctgtgatggaaattgtgctgcaaagtgcgcagggccgttggcccgacgacaagtatttttcgtcgagagaagcaacgaaagcccactcctccaagccacccttcgctcaagtcatatttagcaccacctcggcagtcgacactgcagcacaccaacttaaatattcatcccgatctccctcactcgggaggtccgagttccactcggttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 243 - DNA, 360 bp, synthetic construct
  source 1..360: mol_type = other DNA; note = Recombinant
aacggactcgagcgagagagactctgcggaccgggccagcatcgatgtatttggtagcccagagcccacgcaaccagacagaggtatgaatgttgtgctttgtggtgcttcagtttagtcccacctcggcggttttcagcggaagggagcagagaaggcttataaaagcagacctcagtcaacataacaattaggaggtccgagttccactcggttattgtactctcaagatttatttttccaaaagggttacttaaatcttgcagaagctacaaagataaggcttcatgccgaaatcaacaccctgtcattttatggcagggtgttttcgttatttaatcaaattcaaattttttttaa

SEQ ID NO: 244 - DNA, 373 bp, synthetic construct
  source 1..373: mol_type = other DNA; note = Recombinant
aggcgttcgcttcatttctcttcactccgtttgttgggccggacatcaagcggctgtctgggccgcgcacgcagtcctcttgactagcactgcgtctggctggctctggctgcttcagctttgctttagttccaaaccgcttgctgtagctaataccagcagggagcgctgctatttaggatggactgggcgctgcgagagaagcaggaggtccgagttccactcggttattgtactctcaagatttatttttccaaaagggttacttaaatcttgcagaagctacaaagataaggcttcatgccgaaatcaacaccctgtcattttatggcagggtgttttcgttatttaatcaaattcaaattttttttaa

SEQ ID NO: 245 - DNA, 348 bp, synthetic construct
  source 1..348: mol_type = other DNA; note = Recombinant
caataagataaagctggtgcaggctctcgacctgaagcccacacactggagagcaacgatataccagaatggagcggcccagtacacgatctgctgggattgctggccagtggccagtcccgcagccgattagcaccacctcggtggcacagacgaacgaacgctaatttaaaagcttagctctggagcttggcaggaggtccgagttccactcggttattgtactctcaagatttatttttccaaaagggttacttaaatcttgcagaagctacaaagataaggcttcatgccgaaatcaacaccctgtcattttatggcagggtgttttcgttatttaattttttt

SEQ ID NO: 246 - DNA, 3131 bp, synthetic construct
  source 1..3131: mol_type = other DNA; note = Recombinant
gaattcctatgataaagttgctctgtaacagaaaacaccatctaggtcgacttacttgcggccgcttacttggcgcgccggaggtccgagttccactcgcaagaatttgcttcttgggaggtccgagttccactcgcaagaatcctctcattaggaggtccgagttccactcgcaagaatattattcgcaagacccttcctctatataaggaagttcatttcatttggagaggacacgctgaaatcaccagtctctctctacaaatctatctctctctattttccggaccgaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggggtaccctcagcgctgtgcctgttgcgatcgcaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 247 - DNA, 200 bp, Glycine max
  source 1..200: mol_type = genomic DNA
atccaagcaaaatacttggaagatacgaaagtgtttgaaatcagttattagtttcacgtttgataaaattgctgatttaaatttttgactgttgctctcggctaggaatgttgcaagcgaagaagtcccacatttgtcagaacattggcaggcagctgaagctcactgtataaaaatggagtacttggatagttgaaagc

SEQ ID NO: 248 - DNA, 200 bp, Glycine max
  source 1..200: mol_type = genomic DNA
acttgtttgaactcaattattagtctcaagttcgaagaatattggataaactcaactgccgatttaaatttgaaaatgttgttcttgtgtagagattttaggagcatctgacaccagtgacaaagtcccacatttgtcagaacactgacaatcagctaatgctgacagtataaaagtggagtacttggaaggttgaaagc

SEQ ID NO: 249 - DNA, 200 bp, Glycine max
  source 1..200: mol_type = genomic DNA
aacgtgtttgaactcaattattagtctcaagtttgaagagtactggataaactcaactgctgcattaaatttgaaactgttagttcttgtttggagattttagaagcatcttacacaagtcagagccccacattcgtcagaacactgacaagcagctaatgctcacagtataaaagttgaatacttagacgtttcaaagc

SEQ ID NO: 250 - DNA, 200 bp, Glycine max
  source 1..200: mol_type = genomic DNA
tagttatctggtttcgttaattgtggttgaagcctgaaggcatccattatccctaactatcctggatggttgcaaatactgtcctaagtactacagaaacaagaagactgacagtgtaacgaagtaccacgtctctcaagagaaataacaagcgttgaagactaaactataaataaaaacattatttcattgtacaaagc

SEQ ID NO: 251 - DNA, 200 bp, Glycine max
  source 1..200: mol_type = genomic DNA
aaataaacccgttgaatgaatggaccgaagacaatctgaatccaaaaaagatagctatcattgcttgtgattgaactggctcatgctctgcatccgaacaaaacttggagacacttataacgaagtcccacattgctgagatgagataacactcgcttcagattttattataaaaaacgcattacatattgtggaaactc

SEQ ID NO: 252 - DNA, 200 bp, Glycine max
  source 1..200: mol_type = genomic DNA
attaattgtggatgatggcatccattatccgtggtatttgttagacttggatggttgctggtcgaccaaacaaatactgtcctaaggactacaaaaacaagaagactgacagtgcaacaaagtatcacatcattcaagagaaataacaagcgctaaagactaaacttttaaaaaaaatgcattattccattgttcaaagc

SEQ ID NO: 253 - DNA, 200 bp, Glycine max
  source 1..200: mol_type = genomic DNA
tgttaattgtgtttgaggacatccattatccaacattagaacaagatttgttagacttggatagttgctaatcgagaagtcccagcaaatactacagaaactagaaagctattgatgtaatgaagtcccacatcgctcaagagaaataacaagcactgaagactgaagtataaaacaagcattatttcattgtgcaaagc

SEQ ID NO: 254 - DNA, 200 bp, Glycine max
  source 1..200: mol_type = genomic DNA
atgaatggaacggcttgagtttcgctcaactgcaggaaaatgtggattgcagacaatctgaatcccaaaaagatagttatccttgcttgttatcagaactagacttggacacacttattacgatggcccatatcgcttagatgagataacactcgcttcagattttattataaaaaatgcattgtatgttgtgtaaactc

SEQ ID NO: 255 - DNA, 200 bp, Glycine max
  source 1..200: mol_type = genomic DNA
gtttcatgattccgatcaaagcaagagcatccagtctcaattttgtcttctcaattcactcattcatcaaaatcagcagttttatgcatcaacaagcatggaatgttgaaccacccatgattaagccccatatcgttgtgttgagataactatcacctgaagttgtcttataaaaaacacatctgaatacttttataatc

SEQ ID NO: 256 - DNA, 200 bp, Glycine max
  source 1..200: mol_type = genomic DNA
agtaaaatatcaacgggaagattataatgtgtttgaactttcattagtctcagatttgaacttttatgttgctgatctaaatttttaaccatgttgctcttggctaggatgttgggatgaattagtcccacatttgtcagaactttgtcaggaagctgaagctcccagtataaaatttgaatacttacattgtacaaagc

SEQ ID NO: 257 - DNA, 200 bp, Glycine max
  source 1..200: mol_type = genomic DNA
attcactcattttcagaattatcagtgtgtctacactctacactctacactcagaaacaagcttgaaacattggtgcccattgtcgaagactccatggctaagtcaaattgtcaccatgactaagtcccatatcgattagaagagaggacaatcactccagagcttattataaaacagacattataacaccgttgtactc

SEQ ID NO: 258 - DNA, 200 bp, Glycine max
  source 1..200: mol_type = genomic DNA
tgtggctgaaggcatcaattatccatagttagaacaaagatttgttagacatggatagttgctggtcgaccaaacaaatactgtcctaaggactacaaaacaagaagaccttcagtgtaacgaagtcccacattgcgcaagagaaataacaagcactgaagactgaagtataaaaacaacattatttcattgaacaaatc

SEQ ID NO: 259 - DNA, 200 bp, Glycine max
  source 1..200: mol_type = genomic DNA
agtaaaatatcaacgggaagattataatgtgtttgaacttttattagtctcaggtttgaacttttatgttgctgatctaaatttttaaccatgttgctcttggctaggatgttgggatgaattagtcccacatttgtcagaactttgtcaggcagctgaagctcccagtataaaatttgaatacctacaatgtacaaagc

SEQ ID NO: 260 - DNA, 200 bp, Glycine max
  source 1..200: mol_type = genomic DNA
agtaaaatatcaacgggaagattataatgtgtttgaacttttattagtctcaggtttgaacttttatgttgctgatctaaatttttaaccatgttgctcttggctaggatgttgggatgaattagtcccacatttgtcaggactttgtcaggcagctgaagctcccagtataaaatttgaatacttacattgtacaaagc

SEQ ID NO: 261 - DNA, 200 bp, Glycine max
  source 1..200: mol_type = genomic DNA
ggtgccccatctggtggcagcagagtattgtaatgtgggtgtttggagactggagtgtccatggatttctgtacggagaaatggaagatttaacacataagggtgcgcgttgaagtactaatatggcccatatcgtttagaagagatagaaataactctatatcttattataaaagaagattttgaagccatgtatactc

SEQ ID NO: 262 - DNA, 200 bp, Glycine max
  source 1..200: mol_type = genomic DNA
tgttcatggatttgttctgtgtggagaaattattgatcgtcacaactacattttctagactctgctttgccattgtagtagaggaattactgtttctctgttccaggcgttacaagtataaacagtcccatatcgtttagaagagatagaaataactctatatcttattataaaagaagattttgaagcatagcaaactc

SEQ ID NO: 263 - DNA, 200 bp, Glycine max
  source 1..200: mol_type = genomic DNA
gttataggggtttagcattaatccagatgggttgtgaaatatgcaagtgtaagtgtaatctaattaggatacgttatagacacaaaggtcacttgatgttcaatgtggaaccacccatgagtgagtcccacatcgctgagttaagataagaatcacctgaagttttattataaaacaaacgtttcaacagttaaaaattc

SEQ ID NO: 264 - DNA, 200 bp, Glycine max
  source 1..200: mol_type = genomic DNA
ttttgcaggaatcaggataatggaagaatgaagaaaagatatagagacgtagaggttgaagagaaggtagtagcagaaaatccaagttcttatgtatgttggcgcttaactccttattgcccaagtcccacaccggcgatgagaaagaatctgcgccaaagacttggctataaaacaaacaatccttgaaattcttaagc

SEQ ID NO: 265 - DNA, 200 bp, Glycine max
  source 1..200: mol_type = genomic DNA
ggtttggactctgtacgcactgtcacatcctaaataagaaaacacatttcgaagaaagaccggatattagaagaattaacatttcaaattgttactattgcaacagcaacgtagtcgccaaagagtcccacatcagtcagtcaagaaagcagtagaacatgtaacagaaataaaagcaacaacaatatactgatcaaagc

SEQ ID NO: 266 - DNA, 200 bp, Glycine max
  source 1..200: mol_type = genomic DNA
ctatgtaacaagtacaatttcacagacccggtgttgttctggtcttttgagaagtggtattgtattgtgcagaatgcatgagttatgtagaaacaagcttgccctgaagagctatcctacctaagtcccacactggtaaggagaaagaatatgcaaaaatagtttgactataaaagaaacagtctatgatagaacaaatc

SEQ ID NO: 267 - DNA, 200 bp, Glycine max
  source 1..200: mol_type = genomic DNA
gtagcttcagattactcgtgtgttaactgggtcatgaatccgtgatccaacgttagaacaagaacaaaacctgaatagaagagaaaggctatttggtggatgttgcagaaatactagcgatgaagtctcacattgctgagaagagaaaacaatgacctgatattttattataaaagacaactctggagccttaaaagctc

SEQ ID NO: 268 - DNA, 400 bp, Glycine max
  source 1..400: mol_type = genomic DNA
aagaatgtccactctcaatcactaagttgttgtttcctttttaattcactcatttttagaattatcagtgttatactgaatctagattgaaacatttgtgcccgaagactccatccatgacaaagccccatatcgtctagaagagatgacaatcactccagggcttatcataaaaaagactttttgcagctgtaacactcaagaatgtccactctcaatcactaagttgttgtttcctttttaattcactcatttttagaattatcagtgttatactgaatctagattgaaacatttgtgcccgaagactccatccatgacaaagccccatatcgtctagaagagatgacaatcactccagggcttatcataaaaaagactttttgcagctgtaacactc

SEQ ID NO: 269 - DNA, 200 bp, Glycine max
  source 1..200: mol_type = genomic DNA
agctgaggaaggtagcttcagattactcgtgtgttaactgggtcatgaatccgtgatccaacgtttgaacaagagcaaaatctgaatagaagagaaaggctgttgcagaaatactagtgatgaagtctcacattgctgagaagagaaaacaatgacctgatattttattataaaacacaactctggagccttaacaactc

SEQ ID NO: 270 - DNA, 200 bp, Glycine max
  source 1..200: mol_type = genomic DNA
gttaatattattactcgtgtgttaactgggtcatgaatccgcgatccaacgttagcacaagaacaaaatctgaatagaagagaaaaggcgatttggtggatatagcagaagaactagtgagaaagtcccacattgctgagaagagaaaacaataacatgatgttttattataaaacgcaactctggagtgtgaacaactc

SEQ ID NO: 271 - DNA, 200 bp, Glycine max
  source 1..200: mol_type = genomic DNA
tctgtggaaagattgaagattcaatacaaaccagaccctgtcttgttttgttttggaattatcagttctctattgcagccagacccagagcaagcgtggatgttgcggaagtactagttatgaagtcccacattgctgagaagagaaaacaatgacctgatgttttattataaaatgcaactctggaacatgaacaactc

SEQ ID NO: 272 - DNA, 200 bp, Glycine max
  source 1..200: mol_type = genomic DNA
agtaaaatatcaacgggaagattataatgtgtttgaactttcattagtctcaggtttgaacttttatgttgctgatctaaatttttaaccatgttgctcttggctaggatgttgggatgaattagtcccacatttgtcagaactttgtcaggcagctgaagctcccagtataaaatttgaatacttacattgtacaaagc

SEQ ID NO: 273 - DNA, 200 bp, Glycine max
  source 1..200: mol_type = genomic DNA
agttcttgtttcttttttaattaattcattttcagaattattatacacaaaaactagcttgaaaaatttgtgcccattgtccaagactccatccatgactaggcgtctaagccgcatcgattaagtcccatatcgcttagaaaatatgacaatcactccagagcttattataaaagagacatttttagactaaggcattc

SEQ ID NO: 274 - DNA, 200 bp, Glycine max
  source 1..200: mol_type = genomic DNA
ctaaattcttgtttcttttttaattcactcattttcagaattattatacacaaactagcttgaaaattttgtgcccattgtcgaagactccaccatgactaagggtctaagccccatcgaataagtcccatatcacttagaaaagatgaaaatgactccagagcttattataaaataaacatttttacactgttatattc

SEQ ID NO: 275 - DNA, 200 bp, Glycine max
  source 1..200: mol_type = genomic DNA
ctggatccaacttataaatcagttatagggggtttagcatgactccagatgggttgtgaaatatgcatgtgagtgcaatcacattaggacacttgaagtttaatgttaaaccacccacgattaagtcccgtatcgatgagtaaagataacaatcatctgaagtatttttataaaacgcacgtttcaaagcatagaaattc

SEQ ID NO: 276 - DNA, 200 bp, Glycine max
  source 1..200: mol_type = genomic DNA
tgataggtgaattaattatttttaatatacgaatgaggaagaaagaaaaactattataacaatctgctaagttggggccgaagttgaagtatcaagcgcacgagtgcctctgtatagtgaaaaaagcccacatcgagcagcttactaagttgaagtaaactctaggctataaaatgagagagctactcgtcagttcaacc

SEQ ID NO: 277 - DNA, 200 bp, Glycine max
  source 1..200: mol_type = genomic DNA
catgttgagatgaaagatcaagtattcgtattgcattgatgagtcagatcattgactttggagatgctctacatagagaagagaaagtgaaggatcaagtgtcattctttgtttctggcgaaggtccacatcgagccgcatactgagggaaagtgagctgcttgtactataaatttcaaaggtgcatctgtaaacaaatc

SEQ ID NO: 278 - DNA, 200 bp, Glycine max
  source 1..200: mol_type = genomic DNA
caagctagttgtcccaagcctgcatagagtgataacagcatgctaataactcccaaagaacacagatgaaaaatcaagtatcaagtgtgtgggtgctaacatttcagattctgactaaataaagcccacatcgaatggtatacttagagctagtaagctgcttacgctataaaatgaaaggctcattgctattgatattc

SEQ ID NO: 279 - DNA, 200 bp, Glycine max
  source 1..200: mol_type = genomic DNA
aacaccatcagaagctatgaagaagaacaaaaggctcctggagatattccatttttcattgattccctatcttcatgatattaacagtgtgggagccttgcctgagtttacatttctgaccaaagcccacatcgactggtattgtaatagcaagtgaactggttatacaataaaaggaaagggctgttagctcattactt

SEQ ID NO: 280 - DNA, 200 bp, Glycine max
  source 1..200: mol_type = genomic DNA
caacaataagttggtacctttagagaaaagacatgctttctgtgtatagcattattagcgcacagttgatagaaaatgaagtattgtatatgggtatgatcgagtgtctatttcttgagcaaagcccacattgagtaatataccaaatagaagtgaactgcttatgctataaaatggaagagctgcatttagttttaagc

SEQ ID NO: 281 - DNA, 200 bp, Glycine max
  source 1..200: mol_type = genomic DNA
atgtgagatgaaaaatcaagtattcataatgggtggacgagactgtgacaacttcattcaggggtattacaggtgactggaaagaaagtattaagtgtgcgggtgctaacttttctgactaaagtccacatcgaagggtataccaagagcaagtaagcagcttatgctataaaatgaaagggtcgtttgttttgttactc

SEQ ID NO: 282 - DNA, 200 bp, Glycine max
  source 1..200: mol_type = genomic DNA
aaagatcaagtattcatattgcattgatgagtgagaccattgactttgtagatgctaccgttgatagaagagaaagtgaaggatcaagtatggtcatttctttgtttccgtcttctaacgaaggtccacatcgagccgtattctgagtgagagtgagctgcttataatataaaattcgaaggtgtttacttactaaaaca

SEQ ID NO: 283 - DNA, 389 bp, Glycine max
  source 1..389: mol_type = genomic DNA
tagagagaatcgttaagaaaaaaataaatagtaaagtaaatgaaaacccaaataatatcattattatgtcaataagtcggagaggatagtaatcaaatggtctatgaggtggtggttcattcaacatatagcacctattcattgttcctaaaacataatttaagaacaaaaacttaaacttaaataataataataaaagagtacatcgaagtatctgtgttctctatccttctgactaacattcatgttgtttgtattcagcaaagggccgtgcaggatttgtgcgtcgcgctccggttagttattgcagtgaccgtctctttagtcccacatcgagtaattatgcttcatacagtctgtttatataacagagatggaacaaactggtt

SEQ ID NO: 284 - protein, 9 aa, synthetic construct
  source 1..9: note = Synthetic peptide
LAGLIDADG

SEQ ID NO: 285 - DNA, 28 bp, synthetic construct
  source 1..28: mol_type = other DNA; note = consensus sequence
agctannatgttntacaaatttctncta

SEQ ID NO: 286 - DNA, 22 bp, synthetic construct
  source 1..22: mol_type = other DNA; note = consensus sequence
ctatttnnttctatagcttttt

SEQ ID NO: 287 - DNA, 16 bp, synthetic construct
  source 1..16: mol_type = other DNA; note = consensus sequence
atccntctangnacaa

SEQ ID NO: 288 - DNA, 20 bp, synthetic construct
  source 1..20: mol_type = other DNA; note = consensus sequence
naggacanagtgtcancnag

SEQ ID NO: 289 - DNA, 71 bp, synthetic construct
  source 1..71: mol_type = other DNA; note = consensus sequence
gcgcgttgaccgtgcanantnannggntagttcnacagaangncntagnggcgtgtgtgatcnaaaaacan

SEQ ID NO: 290 - DNA, 53 bp, synthetic construct
  source 1..53: mol_type = other DNA; note = consensus sequence
cntnannggcntngccnnaagaaacatgggccanggcccannatncaangcac

SEQ ID NO: 291 - DNA, 48 bp, synthetic construct
  source 1..48: mol_type = other DNA; note = consensus sequence
cgcnnncaagcccanataccagttcgtnggtggagcaancgaggcgct

SEQ ID NO: 292 - DNA, 187 bp, synthetic construct
  source 1..187: mol_type = other DNA; note = consensus sequence
aacagncaaacatttngtnccacctngnccagncacnattgcnnannnnggcttataagncganncgcaacgcaccncacngtctcttcggagacatccgataaaattggaacgatacagagaagattagcatggcccctgcgcaaggatgacacgcacaaatcgagaaatggtccaaatttttttg

SEQ ID NO: 293 - DNA, 24 bp, synthetic construct
  source 1..24: mol_type = other DNA; note = blunt-end oligonucleotide
agaagtcctcaagtaccgtttggc

SEQ ID NO: 294 - DNA, 14 bp, synthetic construct
  source 1..14: mol_type = other DNA; note = blunt-end oligonucleotide
aagtcctcaaggga

SEQ ID NO: 295 - DNA, 14 bp, synthetic construct
  source 1..14: mol_type = other DNA; note = blunt-end oligonucleotide
gctagtaccgtttg

(19) United States
(12) Patent Application Publication
(10) Pub. No.: US 2026/0242811 A1
(43) Pub. Date: Aug. 20, 2026

(54) COMPOSITIONS AND METHODS FOR SITE DIRECTED GENOMIC MODIFICATION

(71) Applicant: Monsanto Technology LLC, St. Louis, MO (US)

(72) Inventors: Brent Brower-Toland, St. Louis, MO (US); Andrei Y. Kouranov, Chesterfield, MO (US); Rosemarie Kuehn, St. Louis, MO (US); Richard J. Lawrence, Kirkwood, MO (US); Ervin D. Nagy, Lake St. Louis, MO (US); Linda Rymarquis, High Ridge, MO (US); Veena Veena, St. Louis, MO (US)

(21) Appl. No.: 19/659,095

(22) Filed: Apr. 27, 2026

Related U.S. Application Data
(62) Division of application No. 18/596,328, filed on Mar. 5, 2024, now Pat. No. 12,668,808.
Application No. 18/596,328 is a division of application No. 18/146,434, filed on Dec. 26, 2022, now Pat. No. 11,952,578.
Application No. 18/146,434 is a division of application No. 17/503,235, filed on Oct. 15, 2021, now Pat. No. 11,566,254.
Application No. 17/503,235 is a division of application No. 15/120,110, filed on Feb. 27, 2015, now Pat. No. 11,186,843.
(60) Provisional application No. 61/945,700, filed on Feb. 27, 2014.

(51) Int. Cl. C12N 15/82

(52) U.S. Cl. CPC C12N 15/8213; C12N 15/8216

(57) ABSTRACT

The disclosure provides novel corn, tomato, and soybean U6, U3, U2, U5, and 7SL snRNA promoters which are useful for CRISPR/Cas-mediated targeted gene modifications in plants. The disclosure also provides methods for us for U6, U3, U2, U5, and 7SL promoters in driving expression of sgRNA polynucleotides which function in a CRISPR/Cas system of targeted gene modification in plants. The disclosure also provides methods of genome modification by insertion of blunt-end DNA fragments at a site of genomic cleavage.

### CROSS REFERENCE TO RELATED APPLICATIONS

This application is a divisional of U.S. patent application Ser. No. 18/596,328, filed Mar. 5, 2024, which is a divisional of U.S. patent application Ser. No. 18/146,434, filed Dec. 26, 2022, now U.S. Pat. No. 11,952,578, which is a divisional of U.S. patent application Ser. No. 17/503,235, filed Oct. 15, 2021, now U.S. Pat. No. 11,566,254, which is a divisional of U.S. patent application Ser. No. 15/120,110, filed Aug. 18, 2016, now U.S. Pat. No. 11,186,843, which is a 371 National Stage application of International Application Serial No. PCT/US2015/018104, filed Feb. 27, 2015, which claims the priority of U.S. Provisional Appl. Ser. No. 61/945,700, filed Feb. 27, 2014, the entire disclosure of which is incorporated herein by reference.

### INCORPORATION OF SEQUENCE LISTING

The sequence listing that is contained in the file named “MONS350USD4_ST26.xml”, which is 392,205 bytes (measured in MS-WINDOWS) and created on Mar. 24, 2026, is filed herewith by electronic submission and incorporated herein by reference.

### BACKGROUND

### Field

The disclosure relates to the field of biotechnology. More specifically, the disclosure provides a method of introducing recombinant blunt-end double-strand DNA fragments into the genome of a plant by introducing a double-strand break in the genome and novel plant promoters beneficial for the expression of, for instance, non-protein-coding small RNAs for CRISPR-mediated genome modification.

### Description of Related Art

Site-specific recombination has potential for application across a wide range of biotechnology-related fields. Meganucleases, zinc finger nucleases (ZFNs), and transcription activator-like effector nucleases (TALENs) containing a DNA-binding domain and a DNA-cleavage domain enable genome modification. While meganucleases, ZFNs, and TALENs, are effective and specific, these technologies require generation through protein engineering of one or more components for each genomic site chosen for modification. Recent advances in application of clustered, regularly interspaced, short palindromic repeats (CRISPR) have illustrated a method of genome modification that may be as robust as the comparable systems (meganucleases, ZFNs, and TALENs), yet has the advantage of being quick to engineer.

The Clustered Regularly Interspersed Short Palindromic Repeats (CRISPRs) system constitutes an adaptive immune system in prokaryotes that targets endonucleolytic cleavage of invading phage. The system is composed of a protein component (Cas) and a guide RNA (gRNA) that targets the protein to a specific locus for endonucleolytic cleavage. This system has been successfully engineered to target specific loci for endonucleolytic cleavage of mammalian, zebrafish, drosophila, nematode, bacteria, yeast, and plant genomes.

### SUMMARY

In one aspect the invention provides a recombinant DNA construct comprising a snRNA promoter selected from the group consisting of: a U6 promoter, a U3 promoter, a U2 promoter, a U5 promoter, and a 7SL promoter; operably linked to a sequence encoding a single-guide RNA (sgRNA), wherein the sequence of said snRNA promoter comprises SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NOs:146-149, SEQ ID NOs:160-201, or SEQ ID NOs:247-283; or a fragment thereof, wherein the fragment is at least 140 bp in length.

In one embodiment the sequence of said U6 promoter may comprise any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NOs:146-149, SEQ ID NOs:160-166, SEQ ID NOs:200-201, or SEQ ID NO:283, or a fragment thereof, wherein the fragment is at least 140 bp in length. In a further embodiment, the sequence of said U6 promoter may comprise SEQ ID NO:7. In another embodiment the sequence of said U6 promoter may comprise a sequence selected from the group consisting of: SEQ ID NO:17, SEQ ID NO:18, SEQ ID NO:19, and SEQ ID NO:20. In yet another embodiment the sequence of said U3 promoter may comprise any of SEQ ID NOs:167-171 or SEQ ID NOs:178-182, or a fragment thereof, wherein the fragment is at least 140 bp in length. In still yet another embodiment the sequence of said U2 promoter comprises any of SEQ ID NOs:183-187, SEQ ID NOs:192-199, or SEQ ID NOs:247-275, or a fragment thereof, wherein the fragment is at least 140 bp in length. In another embodiment the sequence of said U5 promoter comprises any of SEQ ID NOs:188-191, or SEQ ID NOs:276-282, or a fragment thereof, wherein the fragment is at least 140 bp in length. In a further embodiment the sequence of said 7SL promoter comprises any of SEQ ID NOs:172-177, or a fragment thereof, wherein the fragment is at least 140 bp in length. The recombinant DNA construct may further comprise a transcription termination sequence.

The recombinant DNA construct may also further comprise a sequence encoding a promoter operably linked to a sequence encoding a clustered, regularly interspaced, short palindromic repeats (CRISPR)-associated Cas endonuclease gene product. In certain embodiments of the recombinant DNA construct, the Cas endonuclease gene product may be further operably linked to a nuclear localization sequence (NLS). Further, in certain embodiments of the contemplated recombinant DNA construct, the sequence encoding said Cas endonuclease may be selected from the group consisting of SEQ ID NO:27, SEQ ID NO:68, and SEQ ID NO:97, SEQ ID NO:119, and SEQ ID NO:136.

Another aspect of the invention provides a recombinant DNA construct comprising a snRNA promoter selected from the group consisting of: a U6 promoter, a U3 promoter, a U2 promoter, a U5 promoter, and a 7SL promoter; operably linked to a sequence specifying a non-coding RNA, wherein the sequence of said snRNA promoter comprises SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NOs:146-149, SEQ ID NOs:160-201 or SEQ ID NOs:247-283, or a fragment thereof, wherein the fragment is at least 140 bp in length. In some embodiments the non-coding RNA is selected from the group consisting of: a microRNA (miRNA), a miRNA precursor, a small interfering RNA (siRNA), a small RNA (22-26 nt in length) and precursor encoding same, a heterochromatic siRNA (hc-siRNA), a Piwi-interacting RNA (piRNA), a hairpin double strand RNA (hairpin dsRNA), a trans-acting siRNA (ta-siRNA), and a naturally occurring antisense siRNA (nat-siRNA).

Certain embodiments if the invention further comprise such a recombinant DNA construct, wherein the sequence of said U3 promoter comprises any of SEQ ID NOs:167-171 and SEQ ID NOs:178-182, or a fragment thereof, wherein the fragment is at least 140 bp in length. In another embodiment of the recombinant DNA construct, the sequence of said U2 promoter comprises any of SEQ ID NOs:183-187, SEQ ID NOs:192-199, or SEQ ID NOs:247-275, or a fragment thereof, wherein the fragment is at least 140 bp in length. In yet another embodiment of the recombinant DNA construct, the sequence of said U5 promoter comprises any of SEQ ID NOs:188-191, or SEQ ID NOs:276-282, or a fragment thereof, wherein the fragment is at least 140 bp in length. Still further, the invention provides an embodiment wherein the sequence of said U6 promoter may comprise any of SEQ ID NOs:1-20, SEQ ID NOs:146-149, SEQ ID NOs:160-166, SEQ ID NOs:200-201, or SEQ ID NO:283, or a fragment thereof, wherein the fragment is at least 140 bp in length. Another embodiment comprises the recombinant DNA construct wherein the sequence of said 7SL promoter comprises any of SEQ ID NOs:172-177, or a fragment thereof, wherein the fragment is at least 140 bp in length.

Another aspect of the invention provides a cell comprising a recombinant DNA construct as described above. In certain embodiments the cell is a plant cell.

The invention further provides a method of introducing a double-strand break in the genome of a cell, comprising introducing in said cell: a) at least one recombinant DNA construct of claim 1; and b) a second recombinant DNA construct comprising a sequence encoding a promoter operably linked to a sequence encoding a clustered, regularly interspaced, short palindromic repeats (CRISPR)-associated Cas endonuclease gene product operably linked to a nuclear localization sequence (NLS). In one embodiment of such a method, the sequence of the U6 promoter comprises SEQ ID NO:7. In another embodiment of the method, the U6 promoter comprises a sequence selected from the group consisting of: SEQ ID NO:17, SEQ ID NO:18, SEQ ID NO:19, and SEQ ID NO:20. In yet another embodiment of the method, the sequence encoding said Cas endonuclease is selected from the group consisting of SEQ ID NO:27, SEQ ID NO:68, and SEQ ID NO:97, SEQ ID NO:119, and SEQ ID NO:136.

The invention further provides a method of introducing a double-strand break in the genome of a cell, comprising introducing to said cell at least one recombinant DNA construct which comprises a recombinant DNA construct comprising a snRNA promoter selected from the group consisting of: a U6 promoter, a U3 promoter, a U2 promoter, a U5 promoter, and a 7SL promoter; operably linked to a sequence encoding a single-guide RNA (sgRNA), wherein the sequence of said snRNA promoter comprises SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NOs:146-149, SEQ ID NOs:160-201, or SEQ ID NOs:247-283; or a fragment thereof, wherein the fragment is at least 140 bp in length, and also further comprises a sequence encoding a promoter operably linked to a sequence encoding a clustered, regularly interspaced, short palindromic repeats (CRISPR)-associated Cas endonuclease gene product.

In certain embodiments of the method, the sequence of said U6 promoter comprises SEQ ID NO:7. In other embodiments the U6 promoter comprises a sequence selected from the group consisting of: SEQ ID NO:17, SEQ ID NO:18, SEQ ID NO:19, and SEQ ID NO:20. In some embodiments of the method the sequence encoding the Cas endonuclease is selected from the group consisting of SEQ ID NO:27, SEQ ID NO:68, and SEQ ID NO:97, SEQ ID NO:119, and SEQ ID NO:136.

Another aspect of the invention provides a method of genome modification comprising: a) introducing a double-strand break at a selected site in the genome of a plant cell, and b) introducing into said plant cell a recombinant blunt-end double-strand DNA fragment, wherein said recombinant blunt-end double-strand DNA fragment is incorporated into said double strand break by endogenous DNA repair. The method may comprise genome modification such as production of a modified linkage block, linking two or more QTLs, disrupting linkage of two or more QTLs, gene insertion, gene replacement, gene conversion, deleting or disrupting a gene, transgenic event selection, transgenic trait donor selection, transgene replacement, or targeted insertion of at least one nucleic acid of interest. In some embodiments of the method the double stranded break is introduced by an endonuclease. In certain embodiments the endonuclease may be selected from the group consisting of: a TALEN endonuclease; a CRISPR endonuclease; a meganuclease comprising a “LAGLIDADG,” (SEQ ID NO:284) “GIY-YIG,” “His-Cys box,” or HNH sequence motif, and a Zinc finger nuclease. In particular embodiments the endonuclease is a TALEN endonuclease and TALEN expression constructs are introduced into the plant cell, wherein about 0.1 pmol of each TALEN expression construct is introduced into the plant cell.

Further, in the method the plant cell may be a protoplast or may have been, or is being, grown in a plant cell culture. In certain embodiments of the method the plant cell is selected from the group consisting of: a soybean plant cell; a corn plant cell; a rice plant cell; a wheat plant cell; a turfgrass plant cell; a cotton plant cell; and a canola plant cell. In other embodiments of the method the recombinant blunt-end double-strand DNA fragment does not comprise a region of homology to the selected site in the genome.

Embodiments of the method are contemplated wherein about 0.03 to about 0.3 fmol of recombinant blunt-end double-strand DNA fragment is introduced into said plant cell. In particular embodiments about 0.15 fmol of recombinant blunt-end double-strand DNA fragment is introduced into said plant cell. Further, the blunt-end double-strand DNA fragment may comprise on the 5′ end, or the 3′ end, or both the 5′ and 3′ ends, a region with microhomology to a sequence comprising one or both ends of said double-strand break in the genome. Some embodiments comprise a method wherein the region of microhomology is selected from a sequence 1 bp, 2 bp, 3 bp, 4, bp, 5 bp, 6 bp, 7 bp, 8 bp, 9 bp, or 10 bp in length. In a particular embodiment of the method the region of microhomology is 3 bp in length.

The method may comprise introduction of a double-strand break in step a) as described above, by providing said cell with an endonuclease designed to target a selected target site in the genome of said cell. Further, the endonuclease may be provided by at least one recombinant DNA construct encoding the endonuclease. In an embodiment, the endonuclease is provided by delivering an mRNA encoding the endonuclease or the endonuclease to the plant cell. In particular embodiments The endonuclease is selected from the group consisting of: a TALEN endonuclease; a Zinc finger endonuclease; a meganuclease; and a CRISPR endonuclease. Additional embodiments may comprise introduction of a double-strand break in step a) by providing said cell with a recombinant DNA construct encoding a promoter operably linked to a sequence encoding a clustered, regularly interspaced, short palindromic repeats (CRISPR)-associated Cas endonuclease gene product and a recombinant DNA construct comprising a U6, U3, U2, U5, or 7SL promoter operably linked to a sequence encoding a single-guide RNA (sgRNA) designed to target a selected target site in the chromosome of said cell. In particular embodiments the Cas endonuclease gene product may be further operably linked to at least one nuclear localization sequence (NLS).

In certain embodiments of the method the sequence of said U6, U3, U2, U5, or 7SL promoter may comprise SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NOs:146-149, SEQ ID NOs:160-201 or SEQ ID NOs:247-283, or a fragment thereof; wherein the fragment is at least 140 bp in length and comprises a transcription termination sequence. In particular embodiments the U6 promoter may comprise a sequence selected from the group consisting of: SEQ ID NOs:1-20, SEQ ID NOs:146-149, SEQ ID NOs:160-166, SEQ ID NOs:200-201, and SEQ ID NO:283, or a fragment thereof, wherein the fragment is at least 140 bp in length comprising a transcription termination sequence. In alternative embodiments the U6 promoter may comprise a sequence selected from the group consisting of: SEQ ID NO: 17, SEQ ID NO:18, SEQ ID NO:19, and SEQ ID NO:20. In further embodiments the sequence of said U3 promoter may comprise any of SEQ ID NOs:167-171 or SEQ ID NOs:178-182, or a fragment thereof, wherein the fragment is at least 140 bp in length. In still further embodiments the sequence of said U5 promoter comprises any of SEQ ID NOs:188-191, or SEQ ID NOs:276-282, or a fragment thereof, wherein the fragment is at least 140 bp in length. Additionally, the sequence of said U2 promoter may comprise any of SEQ ID NOs:183-187, SEQ ID NOs:192-199, or SEQ ID NOs:247-275, or a fragment thereof, wherein the fragment is at least 140 bp in length. In yet other embodiments the sequence of said 7SL promoter comprises any of SEQ ID NOs:172-177, or a fragment thereof, wherein the fragment is at least 140 bp in length.

Embodiments are also contemplated wherein the recombinant DNA construct encoding a promoter operably linked to a sequence encoding a clustered, regularly interspaced, short palindromic repeats (CRISPR)-associated Cas endonuclease gene product, and the recombinant DNA construct comprising a U6, U3, U2, U5, or 7SL promoter operably linked to a sequence encoding a single-guide RNA (sgRNA) is designed to target a selected target site in the chromosome of said cell, are on the same construct. Other embodiments of the method may comprise use of a recombinant DNA construct encoding a promoter operably linked to a sequence encoding a clustered, regularly interspaced, short palindromic repeats (CRISPR)-associated Cas endonuclease gene product and the recombinant DNA construct comprising a U6, U3, U2, U5, or 7SL promoter is operably linked to a sequence encoding a single-guide RNA (sgRNA) designed to target a selected target site in the chromosome of said cell are on at least two constructs.

A further aspect of the invention comprises a plant cell comprising a targeted recombinant sited-directed integration of a blunt-end double-strand DNA fragment. Further provided are a plant, plant part, or plant seed comprising a targeted recombinant sited-directed integration of a blunt-end double-strand DNA fragment.

A still further aspect of the invention comprises: a method of genome modification comprising: a) introducing a double-strand break in the genome of a plant cell by introducing a double-strand break in the genome of a cell, comprising introducing in said cell: a) at least one recombinant DNA construct of claim 1; and b) a second recombinant DNA construct comprising a sequence encoding a promoter operably linked to a sequence encoding a clustered, regularly interspaced, short palindromic repeats (CRISPR)-associated Cas endonuclease gene product operably linked to a nuclear localization sequence (NLS); and b) introducing into said plant cell a recombinant blunt-end double-strand DNA fragment, wherein said recombinant blunt-end double-strand DNA fragment is incorporated into said double strand break by endogenous DNA repair.

A further aspect of the invention comprises a method of genome modification comprising: a) introducing a double-strand break in the genome of a plant cell as described above, and b) introducing into said plant cell a recombinant blunt-end double-strand DNA fragment, wherein said recombinant blunt-end double-strand DNA fragment is incorporated into said double strand break by endogenous DNA repair.

Yet another aspect of the invention comprises a recombinant DNA construct comprising at least a first expression cassette comprising a U6, U3, U2, U5, or 7SL promoter operably linked to a sequence encoding a single-guide RNA (sgRNA), wherein the sequence of said promoter comprises any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NOs:146-149, SEQ ID NOs:160-201, or SEQ ID NOs:247-283, or a fragment thereof, wherein the fragment is at least 140 bp in length. In certain embodiments the recombinant DNA construct further comprises at least a second expression cassette, wherein the sequence encoding the first sgRNA is distinct from the sequence encoding the second sgRNA. The recombinant DNA construct may also comprise a construct wherein the promoter operably linked to the sequence encoding the first sgRNA is distinct from the promoter operably linked to the sequence encoding the second sgRNA. In certain embodiments the construct comprises flanking left and right homology arms (HA) which are each about 200-1200 bp in length. In particular embodiments the homology arms are about 230 to about 1003 bp in length.

Another aspect of the invention provides a method of quantifying the activity of a nuclease by detecting integrated DNA fragments by determining the rate of homologous recombination (HR) mediated targeted integration by use of using digital PCR or quantitative PCR.

Yet another aspect of the invention comprises a recombinant DNA construct comprising: a) a first snRNA promoter selected from the group consisting of: a U6 promoter, a U3 promoter, a U2 promoter, a U5 promoter, and a 7SL promoter; operably linked to a sequence encoding a non-coding RNA, and b) a second snRNA promoter selected from the group consisting of: a U6 promoter, a U3 promoter, a U2 promoter, a U5 promoter, and a 7SL promoter; operably linked to a sequence encoding a non-coding RNA, wherein the first snRNA promoter and the second snRNA promoter are different. In certain embodiments the sequence encoding the first snRNA promoter and the sequence encoding the second snRNA promoter each comprise SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO: 14, SEQ ID NO:15, SEQ ID NO:16, SEQ ID NO:17, SEQ ID NO:18, SEQ ID NO:19, SEQ ID NO:20, SEQ ID NOs:146-149, SEQ ID NOs:160-201, or SEQ ID NOs:247-283, or a fragment thereof, wherein the fragment is at least 140 bp in length. Further, a recombinant DNA construct, wherein the first and second snRNA promoter are U6 promoters and the sequences encoding the first and second snRNA promoters are each selected from the group consisting of: SEQ ID NOs:1-8, SEQ ID NOs:17-20, and SEQ ID NOs:200-201 is also provided in certain embodiments.

Thus, a recombinant DNA construct wherein the first and second snRNA promoter are U6 promoters and the sequences encoding the first and second snRNA promoters are each selected from the group consisting of: SEQ ID NOs:12-16, SEQ ID NOs:160-166, and SEQ ID NO:283, is also provided. Alternatively, a recombinant DNA construct, wherein the first and second snRNA promoter are U6 promoters and the sequences encoding the first and second snRNA promoters are each selected from the group consisting of: SEQ ID NOs:9-11 and SEQ ID NO:146-149, is provided.

A recombinant DNA construct, wherein the first and second snRNA promoter are U2 promoters and the sequences encoding the first and second snRNA promoters are each selected from the group consisting of SEQ ID NOs:183-187 and SEQ ID NOs:192-199 is also contemplated. Additionally, certain embodiments of the invention comprise a recombinant DNA construct wherein the first and second snRNA promoter are U2 promoters and the sequences encoding the first and second snRNA promoters are each selected from the group consisting of SEQ ID NOs:247-275.

Yet other embodiments comprise a recombinant DNA construct, wherein the first and second snRNA promoter are U3 promoters and the sequences encoding the first and second snRNA promoters are each selected from the group consisting of SEQ ID NOs:178-182. Still other embodiments of the invention comprise a recombinant DNA construct, wherein the first and second snRNA promoter are U3 promoters and the sequences encoding the first and second snRNA promoters are each selected from the group consisting of SEQ ID NOs:167-171.

Alternatively, the recombinant DNA construct may comprise first and second snRNA promoter which are U5 promoters and wherein the sequences encoding the first and second snRNA promoters are each selected from the group consisting of SEQ ID NOs:188-191. Alternatively provided are recombinant DNA constructs wherein the first and second snRNA promoter are U5 promoters and the sequences encoding the first and second snRNA promoters are each selected from the group consisting of SEQ ID NOs:276-282.

Certain embodiments of the invention provide a recombinant DNA construct wherein the first and second snRNA promoter are 7SL promoters and the sequences encoding the first and second snRNA promoters are each selected from the group consisting of SEQ ID NOs:175-177. In other embodiments the recombinant DNA construct wherein the first and second snRNA promoter are 7SL promoters and the sequences encoding the first and second snRNA promoters are each selected from the group consisting of SEQ ID NOs:172-174.

Also contemplated are embodiments wherein the recombinant DNA construct comprises a first snRNA promoter which is a U6 promoter and a second snRNA promoter is also present and is selected from the group consisting of: a U3 promoter, a U2 promoter, a U5 promoter, and a 7SL promoter. Other embodiments include a recombinant DNA construct wherein the first snRNA promoter is a U3 promoter and the second snRNA promoter is selected from the group consisting of: a U6 promoter, a U2 promoter, a U5 promoter, and a 7SL promoter. Alternatively in the recombinant DNA construct, the first snRNA promoter is a U2 promoter and the second snRNA promoter may be selected from the group consisting of: a U6 promoter, a U3 promoter, a U5 promoter, and a 7SL promoter; or the first snRNA promoter is a U5 promoter and the second snRNA promoter is selected from the group consisting of: a U6 promoter, a U2 promoter, a U3 promoter, and a 7SL promoter. Further, the recombinant DNA construct may comprise a first snRNA promoter which is a 7SL promoter and the second snRNA promoter may be selected from the group consisting of: a U6 promoter, a U2 promoter, a U3 promoter, and a U5 promoter.

Other contemplated embodiments of the invention include a recombinant DNA construct as described above, wherein the sequences encoding the first and second snRNA promoters are each selected from the group consisting of: SEQ ID NOs:1-8, SEQ ID NOs:17-20, SEQ ID NOs:200-201, SEQ ID NOs:183-187, SEQ ID NOs:192-199, SEQ ID NOs:178-182, SEQ ID NOs:188-191, and SEQ ID NOs:175-177. In certain embodiments of the recombinant DNA construct, the sequences encoding the first and second snRNA promoters are each selected from the group consisting of: SEQ ID NOs:12-16, SEQ ID NOs:160-166, SEQ ID NO:283, SEQ ID NOs:247-275, SEQ ID NOs:167-171, SEQ ID NOs:276-282, and SEQ ID NOs:172-174.

The recombinant DNA construct may further comprise a sequence specifying one or more additional snRNA promoters selected from the group consisting of: a U6 promoter, a U3 promoter, a U2 promoter, a U5 promoter, and a 7SL promoter; operably linked to a sequence encoding a non-coding RNA, wherein the first snRNA promoter, the second snRNA promoter, and each of the one or more additional snRNA promoters are different. In particular embodiments of the recombinant DNA construct, the sequence specifying said one or more additional snRNA promoters is selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NOs:146-149, SEQ ID NOs:160-201, or SEQ ID NOs:247-283; or a fragment thereof, wherein the fragment is at least 140 bp in length. Further, the recombinant DNA construct may comprise 3, 4, 5, 6, 7, 8, 9 or 10 snRNA promoters.

In some embodiments of the recombinant DNA construct, the non-coding RNAs are sgRNAs targeting different selected target sites in a chromosome of a plant cell. The recombinant DNA constructs may further comprise a sequence encoding a promoter operably linked to a sequence encoding a clustered, regularly interspaced, short palindromic repeats (CRISPR)-associated Cas endonuclease gene product.

Yet another aspect of the invention provides a method of genome modification comprising: a) introducing double-strand breaks at two or more selected sites in the genome of a plant cell by providing said cell with a clustered, regularly interspaced, short palindromic repeats (CRISPR)-associated Cas endonuclease and a recombinant DNA construct wherein the non-coding RNAs are sgRNAs targeting different selected target sites in a chromosome of a plant cell, and b) introducing into said plant cell one or more exogenous double-strand DNA fragment; wherein said exogenous double-strand DNA fragments are incorporated into said double strand breaks by endogenous DNA repair. In some embodiments said one or more exogenous double-strand DNA fragments are blunt-ended. In certain embodiments of the method, said one or more exogenous double-strand DNA fragments comprise a region of homology to a selected site in the genome. In other embodiments the exogenous double-strand DNA fragments comprise regions of homology to different selected sites in the genome.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1B: Nucleotide sequence alignment of four native corn U6 small nuclear RNA (snRNA) genes, including their putative promoters from chromosomes 1, 2, 3, and 8. (FIG. 1A) and (FIG. 1B) The sequence consensus, and (SEQ ID NOs:285-292) percent conservation are presented below the alignments. (FIG. 1B) The thick arrow indicates the transcription start site; upstream from the transcriptional start site are a ‘TATA Box’, an Upstream Sequence Element (USE), and Monocot-Specific Promoter (MSP) elements, each marked with heavy lined boxes; the stretch of seven thymidine bases (poly-T) at the 3′ end is the transcription termination signal. The sequences in FIG. 1.A and FIG. 1.B correspond the following: ZmU6_Ch1 represented by SEQ ID NO:98; ZmU6_Ch2 represented by SEQ ID NO:99; ZmU6_Ch3 represented by SEQ ID NO:100; ZmU6_Ch8 represented by SEQ ID NO:101.

FIG. 2: Illustration of a modified GUS (0-glucuronidase) reporter gene harboring a direct repeat of the coding sequence (GUUS) interrupted by a target site (TS) for CRISPR cleavage.

FIG. 3: GUS activities detected in corn callus after co-bombardment of a GUUS reporter construct together with CRISPR constructs designed for introducing a double-stranded break (DSB) at the Zm7 genomic target site.

FIG. 4: GUS activity detected in corn callus after co-bombardment of a GUUS reporter construct together with CRISPR constructs designed for introducing a DSB at the Zm231 genomic target site. A different genomic target and single-guide RNA (sgRNA) spacer sequence, Zm14, were used as negative control. Also shown are fluorescence microscopy images of representative calli which were co-bombarded with a green fluorescent protein (GFP) expression vector with the GUUS reporter construct, Cas9 expression vector and vectors containing the various sgRNA cassettes.

FIGS. 5A-5E: Illustrations of (FIG. 5A) oligonucleotide integration assay; (FIG. 5B) blunt-end oligonucleotide without microhomology used for insertion at a corn genomic target site (SEQ ID NO:293 pre-insertion and SEQ ID NOs:294 and 295 after insertion; (FIG. 5C) blunt-end oligonucleotide with microhomology ends used for insertion at a corn genomic target site (SEQ ID NO:293 pre-insertion and SEQ ID NOs:294 and 295 after insertion; (FIG. 5D) fragment analysis profile of PCR amplicons spanning the oligo-chromosome junction in test (upper panel) and negative control samples (bottom panel) of the oligonucleotide integration assay (where the arrow indicates the expected peak); and (FIG. 5E) DNA sequences of oligonucleotide-chromosome junctions (SEQ ID NOs:294 and 295) at the Zm_L70c corn genomic target site confirming integrations of both full-length (integration 1; SEQ ID NO:103) and truncated oligonucleotides (integration 2; SEQ ID NO:104), the expected sequence_(template) is presented as SEQ ID NO:102.

FIG. 6: Illustration of a sgRNA including a spacer sequence complementary to a native corn genomic target site and an artificial loop (5′-CCAAAAGG-3′; SEQ ID NO:105) and its predicted secondary structure designed for Streptococcus thermophilus Cas9-mediated targeting (SEQ ID NO:110).

FIGS. 7A-7B: Illustrations of (FIG. 7A) selectable marker gene removal by multiplex CRISPR activity following targeted integration of the gene of interest (GOI); and (FIG. 7B) a CRISPR/Cas multiplex system to evaluate gene linkage of multiple QTL candidate genes. Where likelihood of odds (LOD) is a statistical measure for genetic linkage; an LOD of 3 means that it is 1000× more likely that a QTL exists in the interval than that there is no QTL.

FIG. 8. Graphical presentation of data showing percentage targeted integration rates (Y-axis) detected at 24 and 48 hours post-transformation of corn protoplasts using CRISPR constructs targeting a native chromosomal target (Zm7) in corn and a titration of the pmol of blunt-end, double-stranded DNA fragment added to the transfection mixture (X-axis). The negative controls were run without added Cas9 expression constructs.

FIG. 9. Graphical presentation of the integration rate (Y-axis) as a function of the amount (in pmol) of SpCas9 expression construct added to transfection mixture of corn protoplasts (X-axis).

FIGS. 10A-10C. Sequence confirmation for targeted integrations of blunt-end, double-strand DNA fragments into chromosomes of corn protoplasts transformed with CRISPR/Cas9 and sgRNA expression constructs. For all panels FIGS. 10A, 10B, 10C, the top sequence is the expected sequence of one junction of the target site and the blunt-end double-strand DNA fragment (underlined sequence) included in the experiment. FIG. 10A. Corn chromosome site Zm7 targeted by CRISPR/Cas9 constructs and with blunt-end double-strand DNA fragment formed by annealed DNA fragments represented by SEQ ID NO:115 and SEQ ID NO:116. FIG. 10B. Corn chromosome site L70c targeted by CRISPR/Cas9 constructs and with blunt-end double-strand DNA fragment without micro-homology sequences formed by annealed DNA fragments represented by SEQ ID NO:45 and SEQ ID NO:46. FIG. 10C. Corn chromosome site L70c targeted by CRISPR/Cas9 constructs and with blunt-end double-strand DNA fragment with 3 bp micro-homology sequences at each end of the DNA fragment formed by annealed DNA fragments represented by SEQ ID NO:121 and SEQ ID NO:122.

FIG. 11. Graphical presentation of the integration rate (Y-axis) as a function of the amount (in pmol) of TALEN expression constructs targeting corn chromosome site L70.4 which were added to transfection mixture of corn protoplasts (X-axis).

FIGS. 12A-12B. Schematic representation of NHEJ and HR-mediated targeted integration and PCR primer positions for high through-put screening. Targeted integration of a DNA fragment by non-homologous end-joining (NHEJ) is presented in FIG. 12A and targeted integration of a DNA fragment by homologous recombination (HR) is presented in FIG. 12B.

FIGS. 13A-13B. Schematic representation of the constructs used for homologous integration. The blunt-end DNA arrow indicates the 90 bp sequence corresponding to the 90 bp blunt-end, double-strand DNA fragment used for NHEJ assays, LHA refers to left-homology arm, RHA refers to right-homology arm, Zm7 refers to the target site Zm7 targeted by a CRISPR/Cas9+sgRNA. The length in bp of each of the homology arms is indicated. FIG. 13A. Schematic for HR-cassette construct for targeting the corn chromosome site Zm7 with LHA and RHA of 240 and 230 bp in length, respectively. FIG. 13B. Schematic for HR-cassette construct for targeting the corn chromosome site Zm7 with LHA and RHA of 240 and 1003 bp in length, respectively.

FIGS. 14A-14B. Schematic representation of the constructs used for homologous integration. In the figure, blunt-end DNA arrow indicates the 90 bp sequence corresponding to the 90 bp blunt-end, double-strand DNA fragment used for NHEJ assays, LHA refers to left-homology arm, RHA refers to right-homology arm, L70.4 refers to the target site L70.4 in the corn chromosome targeted by a TALEN pair. The length in bp of each of the homology arms is indicated. FIG. 14A. Schematic for HR-cassette construct for targeting the corn chromosome site L70.4 with both the LHA and RHA 230 bp in length. FIG. 14B. Schematic for HR-cassette construct for targeting the corn chromosome site L70.4 with LHA and RHA of 1027 bp and 230 bp in length, respectively.

FIGS. 15A-15B. FIG. 15A. Graphical presentation of data showing percent targeted integration rates in transfected corn protoplasts using StCas9 CRISPR constructs targeting native corn chromosomal target sites L70e, L70f, and L70g. The controls lacked a StCas9 expression cassette construct in the transfection mixture. FIG. 15B. Sequence alignment of expected integration of the blunt-end, double-strand DNA fragment at the L70f target site (SEQ ID NO:144) and one example of target site integration with indel of the DNA fragment sequence (SEQ ID NO:145).

FIGS. 16A-16B. FIG. 16A. Chromosomal integration rates using constructs with the corn chromosome 8 U6 promoter or one of three separate chimeric U6 promoters driving sgRNA expression in CRISPR/Cas9 system to target three different corn chromosomal target sites. Targeted integration was measured by ddPCR assay using MGB TaqMan probes. FIG. 16B. Chromosomal integration rates using constructs with the corn chromosome 8 U6 promoter or one of three separate chimeric U6 promoters driving sgRNA expression in CRISPR/Cas9 system to target three different corn chromosomal target sites. Targeted integration was measured by ddPCR assay using EvaGreen® intercalating dye.

FIGS. 17A-17C. FIG. 17A. Schematic of PCR screening strategy to detect CRISPR/Cas9 induced mutation by NHEJ at tomato invertase inhibitor target site 2 (TS2), resulting in mutation of restriction endonuclease site SmlI. FIG. 17B. Photograph of PCR amplicons run on an agarose gel showing undigested amplicons and SmlI digested amplicons to detect CRISPR/Cas9 induced mutation at tomato invertase inhibitor target site 2. FIG. 17C. Multiple sequence alignment of sequences of PCR amplicons from CRISPR/Cas9 induced mutation by NHEJ at the tomato invertase inhibitor target site 2.

FIGS. 18A-18B. FIG. 18A. Graphical representation of data showing normalized GUS mRNA levels from soybean cotyledon protoplast assays with recombinant expression constructs with U6, U3, and 7SL promoters. FIG. 18B. Graphical representation of data showing normalized GUS mRNA levels from corn leaf protoplast assays with recombinant expression constructs with U6, U3, 7SL, U2, or U5 promoters.

FIG. 19. Graphical representation of data from normalized GUS expression levels from corn leaf protoplast assays with, a recombinant expression constructs encoding 1) a GUS expression construct 2) a dead Cas9-TALE-AD expression construct, and 3) recombinant sgRNA expression constructs with 7SL, U6, U3, U2, or U5 promoters.

### DETAILED DESCRIPTION

The disclosure provides novel promoters from Zea mays and other plants, and methods for their use that include targeted gene modification of a plant genome using transgenic expression of a gene, or genes, involved in the Clustered Regularly Interspersed Short Palindromic Repeats (CRISPR) system found in many bacteria. For instance, the disclosure provides, in one embodiment, DNA constructs encoding at least one expression cassette including a U6 promoter disclosed herein and a sequence encoding a single-guide RNA (sgRNA). Methods for causing a CRISPR system to modify a target genome are also provided, as are the genomic complements of a plant modified by the use of such a system. The disclosure thus provides tools and methods that allow one to insert, remove, or modify genes, loci, linkage blocks, and chromosomes within a plant. Also disclosed are U3, U2, U5 and 7SL promoters and methods for their use that include targeted gene modification of a plant genome.

The disclosure provides, in another embodiment, DNA constructs encoding at least one expression cassette including a promoter disclosed herein and a sequence encoding a non-protein-coding small RNA (npcRNA). These constructs are useful for targeting nuclear expression of the npcRNA molecules.

The CRISPR system constitutes an adaptive immune system in prokaryotes that targets endonucleolytic cleavage of the DNA and RNA of invading phage (reviewed in Westra et al., Annu Rev Genet, 46:311-39, 2012). There are three known types of CRISPR systems, Type I, Type II, and Type III. The CRISPR systems rely on small RNAs for sequence-specific detection and targeting of foreign nucleic acids for destruction. The components of the bacterial CRISPR systems are CRISPR-associated (Cas) genes and CRISPR array(s) consisting of genome-target sequences (protospacers) interspersed with short palindromic repeats. Transcription of the protospacer/repeat elements into precursor CRISPR RNA (pre-crRNA) molecules is followed by enzymatic cleavage triggered by hybridization between a trans-acting CRISPR RNA (tracrRNA) molecule and a pre-crRNA palindromic repeat. The resulting crRNA:tracrRNA molecules, consisting of one copy of the spacer and one repeat, complex with a Cas nuclease. The CRISPR/Cas complex is then directed to DNA sequences (protospacer) complementary to the crRNA spacer sequence, where this RNA-Cas protein complex silences the target DNA through enzymatic cleavage of both strands (double-strand break; DSB).

The native bacterial type II CRISPR system requires four molecular components for targeted cleavage of exogenous DNAs: a Cas endonuclease (e.g., Cas9), the house-keeping RNaseIII, CRISPR RNA (crRNA) and trans-acting CRISPR RNA (tracrRNA). The latter two components form a dsRNA complex and bind to Cas9 resulting in an RNA-guided DNA endonuclease complex. For targeted genome modifications in eukaryotes, this system was simplified to two components: the Cas9 endonuclease and a chimeric crRNA-tracrRNA, called guide-RNA (gRNA) or, alternatively, single-guide RNA (sgRNA). Experiments initially conducted in eukaryotic systems determined that the RNaseIII component was not necessary to achieve targeted DNA cleavage. The minimal two component system of Cas9 with the sgRNA, as the only unique component, enables this CRISPR system of targeted genome modification to be more cost effective and flexible than other targeting platforms such as meganucleases, Zn-finger nucleases, or TALE-nucleases which require protein engineering for modification at each targeted DNA site. Additionally, the ease of design and production of sgRNAs provides the CRISPR system with several advantages for application of targeted genome modification. For example, the CRISPR/Cas complex components (Cas endonuclease, sgRNA, and, optionally, exogenous DNA for integration into the genome) designed for one or more genomic target sites can be multiplexed in one transformation, or the introduction of the CRISPR/Cas complex components can be spatially and/or temporally separated.

Expression Strategies for sgRNAs

The disclosure provides, in certain embodiments, novel combinations of promoters and a sequence encoding a sgRNA, to allow for specifically introducing a double-stranded DNA cleavage event into endogenous DNA (i.e., a genome). In one embodiment, a U6 promoter from corn is operably linked to a sgRNA-encoding gene, in order to constitutively express the sgRNA in transformed cells. This may be desirable, for example, when the resulting sgRNA transcripts are retained in the nucleus and will thus be optimally located within the cell to guide nuclear processes. This may also be desirable, for example, when the activity of the CRISPR is low or the frequency of finding and cleaving the target site is low. It may also be desirable when a promoter for a specific cell type, such as the germ line, is not known for a given species of interest. In another embodiment, a U3, U2, U5, or 7SL promoter is operably linked to a sgRNA-encoding gene, for expression of an sgRNA in transformed cells.

In another embodiment, a chimeric promoter comprising all or a portion of any of the U6 promoters provided herein can be used to express a sgRNA. Alternatively, a U3, U2, U5, or 7SL chimeric promoter comprising all or a portion of any of these promoters, may be utilized. For example, the 5′ portion of the U6 promoter from corn chromosome 1 (SEQ ID NO:1), including one MSP element, operably linked to the 3′ portion of the U6 promoter from corn chromosome 8 (SEQ ID NO:7), including a USE element and a TATA box (SEQ ID NO:17), cloned upstream of a sgRNA, may be used to induce CRISPR-mediated cleavage under different environmental conditions.

Multiple U6 promoters with differing sequence may be utilized to minimize problems in vector stability, which is typically associated with sequence repeats. Further, highly repetitive regions in chromosomes may lead to genetic instability and silencing. Therefore, use of multiple U6 (or other disclosed) promoters in the CRISPR/Cas system of targeted gene modification may facilitate vector stacking of multiple sgRNA cassettes in the same transformation construct, wherein the differing sgRNA transcript levels are to be optimized for efficient targeting of a single target site. Chimeric U6 promoters can result in new, functional versions with improved or otherwise modified expression levels, and four representative chimeric corn U6 promoters have been designed (SEQ ID NO:17, SEQ ID NO:18, SEQ ID NO:19, and SEQ ID NO:20).

The disclosed U6 promoters may also drive expression of other non-protein-coding RNA (npcRNA). Non-limiting examples of non-protein-coding small RNA include a microRNA (miRNA), a miRNA precursor, a small interfering RNA (siRNA), a small RNA (22-26 nt in length) and precursor encoding same, a heterochromatic siRNA (hc-siRNA), a Piwi-interacting RNA (piRNA), a hairpin double strand RNA (hairpin dsRNA), a trans-acting siRNA (ta-siRNA), and a naturally occurring antisense siRNA (nat-siRNA).

Promoters and transcriptional elements for additional small nuclear RNA (snRNA) genes, similar to U6 promoters and which may be transcribed by RNA polymerase II or RNA polymerase III, can also be identified, such as U3, U2, U5, and 7SL promoters. These alternate promoters can be useful in cassette design, especially where these additional elements may facilitate nuclear retention of the CRISPR system transcripts. Additional gene transcription elements that can be useful in CRISPR cassette design include intron-embedded elements and transcriptional elements of plant specific RNA polymerase IV and V promoters.

### Expression Strategies for Cas-Associated Genes

The disclosure provides novel promoters for use in sequence-specific or sequence-directed CRISPR-mediated cleavage for molecular breeding by providing transcription of, for example, a sgRNA including a spacer sequence used to target a protospacer sequence within a genomic target site for endonuclease cleavage by at least one Cas protein, wherein the genomic target site is native or transgenic. In addition, CRISPR systems can be customized to catalyze cleavage at one or more genomic target sites. In certain embodiments, such a custom CRISPR system would have properties making it amenable to genetic modification such that the system's Cas endonuclease protein(s) recognition, binding and/or catalytic activity could be manipulated.

One aspect of this disclosure is to introduce into a plant cell an expression vector comprising one or more cassettes encoding a U6 corn promoter, or other disclosed promoter such as an U3, U2, U5 or 7SL promoter, operably linked to a sgRNA, including a copy of a spacer sequence complementary to a protospacer sequence within a genomic target site, and an expression vector encoding a Cas-associated gene to modify the plant cell in such a way that the plant cell, or a plant comprised of such cells, will subsequently exhibit a beneficial trait. In one non-limiting example, the trait is a trait such as improved yield, resistance to biotic or abiotic stress, herbicide tolerance, or other improvements in agronomic performance. The ability to generate such a plant cell derived therefrom depends on introducing the CRISPR system using transformation vectors and cassettes described herein.

The expression vector encoding a Cas-associated gene may comprise a promoter. In certain embodiments, the promoter is a constitutive promoter, a tissue specific promoter, a developmentally regulated promoter, or a cell cycle regulated promoter. Certain contemplated promoters include ones that only express in the germline or reproductive cells, among others. Such developmentally regulated promoters have the advantage of limiting the expression of the CRISPR system to only those cells in which DNA is inherited in subsequent generations. Therefore, a CRISPR-mediated genetic modification (i.e., chromosomal or episomal dsDNA cleavage) is limited only to cells that are involved in transmitting their genome from one generation to the next. This might be useful if broader expression of the CRISPR system were genotoxic or had other unwanted effects. Examples of such promoters include the promoters of genes encoding DNA ligases, recombinases, replicases, and so on.

Endonucleases are enzymes that cleave the phosphodiester bond within a polynucleotide chain. Examples of endonucleases that cleave only at specific nucleotide sequences are well known in the art and can include, for instance, restriction endonucleases. However, the need for targeted genome engineering as an alternative to classical plant breeding requires highly customizable tools for genome editing. The CRISPR-associated type II prokaryotic adaptive immune system provides such an alternative. As such, the DNA constructs provided herein can recognize a specific nucleotide sequence of interest within a target host genome and allow for mutation or integration at that site. In a particular embodiment, the DNA constructs contain one or more corn U6 promoter, or chimeras thereof, that express high levels of a sequence encoding a sgRNA. A DNA construct that expresses a sgRNA that targets a Cas-associated gene product with endonuclease activity to a specific genomic sequence, such that the specific genomic sequence is cleaved and produces a double-stranded break which is repaired by a double strand break repair pathway, which may include, for example, non-homologous end-joining, homologous recombination, synthesis-dependent strand annealing (SDSA), single-strand annealing (SSA), or a combination thereof thereby disrupting the native locus, may be particularly useful.

In one embodiment, a CRISPR system comprises at least one Cas-associated gene encoding a CRISPR endonuclease and one sgRNA comprising a copy of a spacer sequence complementary to a protospacer sequence within an endogenous genomic target site.

In particular embodiments, a Cas-associated gene can include any type II CRISPR system endonuclease. Such a Cas-associated gene product would have properties making it amenable to genetic modification such that its nuclease activity and its recognition and binding of crRNA, tracrRNA, and/or sgRNA could be manipulated.

The present disclosure also provides for use of CRISPR-mediated double-stranded DNA cleavage to genetically alter expression and/or activity of a gene or gene product of interest in a tissue- or cell-type specific manner to improve productivity or provide another beneficial trait, wherein the nucleic acid of interest may be endogenous or transgenic in nature. Thus, in one embodiment, a CRISPR system is engineered to mediate disruption at specific sites in a gene of interest. Genes of interest include those for which altered expression level/protein activity is desired. These DNA cleavage events can be either in coding sequences or in regulatory elements within the gene.

This disclosure provides for the introduction of a type II CRISPR system into a cell. Exemplary type II Cas-associated genes include natural and engineered (i.e., modified, including codon-optimized) nucleotide sequences encoding polypeptides with nuclease activity such as Cas9 from Streptococcus pyogenes, Streptococcus thermophilus, or Bradyrhizobium sp.

The catalytically active CRISPR-associate gene (e.g., Cas9 endonuclease) can be introduced into, or produced by, a target cell. Various methods may be used to carry this out, as disclosed herein.

### Transient Expression of CRISPRs

In some embodiments, the sgRNA and/or Cas-associated gene is transiently introduced into a cell. In certain embodiments, the introduced sgRNA and/or Cas-associated gene is provided in sufficient quantity to modify the cell but does not persist after a contemplated period of time has passed or after one or more cell divisions. In such embodiments, no further steps are needed to remove or segregate the sgRNA and/or Cas-associated gene from the modified cell. In yet other embodiments of this disclosure, double-stranded DNA fragments are also transiently introduced into a cell along with sgRNA and/or Cas-associated gene. In such embodiments, the introduced double-stranded DNA fragments are provided in sufficient quantity to modify the cell but do not persist after a contemplated period of time has passed or after one or more cell divisions.

In another embodiment, mRNA encoding the Cas-associated gene is introduced into a cell. In such embodiments, the mRNA is translated to produce the type II CRISPR system endonuclease in sufficient quantity to modify the cell (in the presence of at least one sgRNA) but does not persist after a contemplated period of time has passed or after one or more cell divisions. In such embodiments, no further steps are needed to remove or segregate the Cas-associated gene from the modified cell.

In one embodiment of this disclosure, a catalytically active Cas-associated gene product is prepared in vitro prior to introduction to a cell, including a prokaryotic or eukaryotic cell. The method of preparing a Cas-associated gene product depends on its type and properties and would be known by one of skill in the art. For example, if the Cas-associated gene product is a large monomeric DNA nuclease, the active form of the Cas-associated gene product can be produced via bacterial expression, in vitro translation, via yeast cells, in insect cells, or by other protein production techniques described in the art. After expression, the Cas-associated gene product is isolated, refolded if needed, purified and optionally treated to remove any purification tags, such as a His-tag. Once crude, partially purified, or more completely purified Cas-associated gene products are obtained, the protein may be introduced to, for example, a plant cell via electroporation, by bombardment with Cas-associated gene product coated particles, by chemical transfection or by some other means of transport across a cell membrane. Methods for introducing nucleic acids into bacterial and animal cells are similarly well known in the art. The protein can also be delivered using nanoparticles, which can deliver a combination of active protein and nucleic acid. Once a sufficient quantity of the Cas-associated gene product is introduced so that an effective amount of in vivo nuclease activity is present, along with the appropriate sgRNA, the protospacer sequences within the episomal or genomic target sites are cleaved. It is also recognized that one skilled in the art might create a Cas-associated gene product that is inactive but is activated in vivo by native processing machinery; such a Cas-associated gene product is also contemplated by this disclosure.

In another embodiment, a construct that will transiently express a sgRNA and/or Cas-associated gene is created and introduced into a cell. In yet another embodiment, the vector will produce sufficient quantities of the sgRNAs and/or Cas-associated gene in order for the desired episomal or genomic target site or sites to be effectively modified by CRISPR-mediated cleavage. For instance, the disclosure contemplates preparation of a vector that can be bombarded, electroporated, chemically transfected or transported by some other means across the plant cell membrane. Such a vector could have several useful properties. For instance, in one embodiment, the vector can replicate in a bacterial host such that the vector can be produced and purified in sufficient quantities for transient expression. In another embodiment, the vector can encode a drug resistance gene to allow selection for the vector in a host, or the vector can also comprise an expression cassette to provide for the expression of the sgRNA and/or Cas-associated gene in a plant. In a further embodiment, the expression cassette could contain a promoter region, a 5′ untranslated region, an optional intron to aid expression, a multiple cloning site to allow facile introduction of a sequence encoding sgRNAs and/or Cas-associated gene, and a 3′ UTR. In particular embodiments, the promoters in the expression cassette would be U6 promoters from Zea mays In yet other embodiments, the promoters would be chimeric U6 promoters from Zea mays. In some embodiments, it can be beneficial to include unique restriction sites at one or at each end of the expression cassette to allow the production and isolation of a linear expression cassette, which can then be free of other vector elements. The untranslated leader regions, in certain embodiments, can be plant-derived untranslated regions. Use of an intron, which can be plant-derived, is contemplated when the expression cassette is being transformed or transfected into a monocot cell.

In other embodiments, one or more elements in the vector include a spacer complementary to a protospacer contained within an episomal or genomic target site. This facilitates CRISPR-mediated modification within the expression cassette, enabling removal and/or insertion of elements such as promoters and transgenes.

In another approach, a transient expression vector may be introduced into a cell using a bacterial or viral vector host. For example, Agrobacterium is one such bacterial vector that can be used to introduce a transient expression vector into a host cell. When using a bacterial, viral or other vector host system, the transient expression vector is contained within the host vector system. For example, if the Agrobacterium host system is used, the transient expression cassette would be flanked by one or more T-DNA borders and cloned into a binary vector. Many such vector systems have been identified in the art (reviewed in Hellens et al., 2000).

In embodiments whereby the sgRNA and/or Cas-associated gene is transiently introduced in sufficient quantities to modify a cell, a method of selecting the modified cell may be employed. In one such method, a second nucleic acid molecule containing a selectable marker is co-introduced with the transient sgRNA and/or Cas-associated gene. In this embodiment, the co-introduced marker may be part of a molecular strategy to introduce the marker at a target site. For example, the co-introduced marker may be used to disrupt a target gene by inserting between genomic target sites. In another embodiment, the co-introduced nucleic acid may be used to produce a visual marker protein such that transfected cells can be cell-sorted or isolated by some other means. In yet another embodiment, the co-introduced marker may randomly integrate or be directed via a second sgRNA:Cas-protein complex to integrate at a site independent of the primary genomic target site. In still yet another embodiment, the co-introduced molecule may be targeted to a specific locus via a double strand break repair pathway, which may include, for example, non-homologous end-joining, homologous recombination, synthesis-dependent strand annealing (SDSA), single-strand annealing (SSA), or a combination thereof, at the genomic target site(s). In the above embodiments, the co-introduced marker may be used to identify or select for cells that have likely been exposed to the sgRNA and/or Cas-associated gene and therefore are likely to have been modified by the CRISPR.

### Stable Expression of CRISPRs

In another embodiment, a CRISPR expression vector is stably transformed into a cell so as to cleave a DNA sequence at or near a genomic target site in the host genome with a sgRNA and Cas-associated gene product encoded within the vector. In this embodiment, the design of the transformation vector provides flexibility for when and under what conditions the sgRNA and/or Cas-associated gene is expressed. Furthermore, the transformation vector can be designed to comprise a selectable or visible marker that will provide a means to isolate or efficiently select cell lines that contain and/or have been modified by the CRISPR.

Cell transformation systems have been described in the art and descriptions include a variety of transformation vectors. For example, for plant transformations, two principal methods include Agrobacterium-mediated transformation and particle gun bombardment-mediated (i.e., biolistic) transformation. In both cases, the CRISPR is introduced via an expression cassette. The cassette may contain one or more of the following elements: a promoter element that can be used to express the sgRNA and/or Cas-associated gene; a 5′ untranslated region to enhance expression; an intron element to further enhance expression in certain cell types, such as monocot cells; a multiple-cloning site to provide convenient restriction sites for inserting the sgRNA and/or Cas-associated gene sequences and other desired elements; and a 3′ untranslated region to provide for efficient termination of the expressed transcript. In particular embodiments, the promoters in the expression cassette would be U6 promoters from Zea mays. In yet other embodiments, the promoters would be chimeric U6 promoters from Zea mays.

For particle bombardment or with protoplast transformation, the expression cassette can be an isolated linear fragment or may be part of a larger construct that might contain bacterial replication elements, bacterial selectable markers or other elements. The sgRNA and/or Cas-associated gene expression cassette(s) may be physically linked to a marker cassette or may be mixed with a second nucleic acid molecule encoding a marker cassette. The marker cassette is comprised of necessary elements to express a visual or selectable marker that allows for efficient selection of transformed cells. In the case of Agrobacterium-mediated transformation, the expression cassette may be adjacent to or between flanking T-DNA borders and contained within a binary vector. In another embodiment, the expression cassette may be outside of the T-DNA. The presence of the expression cassette in a cell may be manipulated by positive or negative selection regime(s). Furthermore, a selectable marker cassette may also be within or adjacent to the same T-DNA borders or may be somewhere else within a second T-DNA on the binary vector (e.g., a 2 T-DNA system).

In another embodiment, cells that have been modified by a CRISPR, either transiently or stably, are carried forward along with unmodified cells. The cells can be sub-divided into independent clonally derived lines or can be used to regenerate independently derived plants. Individual plants or clonal populations regenerated from such cells can be used to generate independently derived lines. At any of these stages a molecular assay can be employed to screen for cells, plants or lines that have been modified. Cells, plants or lines that have been modified continue to be propagated and unmodified cells, plants or lines are discarded. In these embodiments, the presence of an active CRISPR in a cell is essential to ensure the efficiency of the overall process.

### Transformation Methods

Methods for transforming or transfecting a cell are well known in the art. Methods for plant transformation using Agrobacterium or DNA coated particles are well known in the art and are incorporated herein. Suitable methods for transformation of host cells for use with the current disclosure are believed to include virtually any method by which DNA can be introduced into a cell, for example by Agrobacterium-mediated transformation (U.S. Pat. Nos. 5,563,055; 5,591,616; 5,693,512; 5,824,877; 5,981,840; and 6,384,301) and by acceleration of DNA coated particles (U.S. Pat. Nos. 5,015,580; 5,550,318; 5,538,880; 6,160,208; 6,399,861; and 6,403,865), etc. Through the application of techniques such as these, the cells of virtually any species may be stably transformed.

Various methods for selecting transformed cells have been described. For example, one might utilize a drug resistance marker such as a neomycin phosphotransferase protein to confer resistance to kanamycin or to use 5-enolpyruvyl shikimate phosphate synthase to confer tolerance to glyphosate. In another embodiment, a carotenoid synthase is used to create an orange pigment that can be visually identified. These three exemplary approaches can each be used effectively to isolate a cell or plant or tissue thereof that has been transformed and/or modified by a CRISPR.

When a nucleic acid sequence encoding a selectable or screenable marker is inserted into a genomic target site, the marker can be used to detect the presence or absence of a CRISPR or its activity. This may be useful once a cell has been modified by a CRISPR, and recovery of a genetically modified cell that no longer contains the CRISPR, or a regenerated plant from such a modified cell, is desired. In other embodiments, the marker may be intentionally designed to integrate at the genomic target site, such that it can be used to follow a modified cell independently of the CRISPR. The marker can be a gene that provides a visually detectable phenotype, such as in the seed, to allow rapid identification of seeds that carry or lack a CRISPR expression cassette.

This disclosure provides for a means to regenerate a plant from a cell with a repaired double-stranded break within a protospacer sequence at a genomic target site. The regenerant can then be used to propagate additional plants.

The disclosure additionally provides novel plant transformation vectors and expression cassettes which include novel U6 promoters, and U3, U2, U5 and 7SL promoters, and combinations thereof, with CRISPR-associated gene(s) and sgRNA expression cassettes. The disclosure further provides methods of obtaining a plant cell, a whole plant, and a seed or embryo that have been specifically modified using CRISPR-mediated cleavage. This disclosure also relates to a novel plant cell containing a CRISPR-associated Cas endonuclease expression construct and sgRNA expression cassettes.

### Targeting Using Blunt-End Oligonucleotides

In certain embodiments, the CRISPR/Cas9 system can be utilized for targeting insertion of a blunt-end double-stranded DNA fragment into a genomic target site of interest. CRISPR-mediated endonuclease activity can introduce a double stand break (DSB) in the protospacer of the selected genomic target site and DNA repair, such as microhomology-driven non-homologous end-joining DNA repair, results in insertion of the blunt-end double-stranded DNA fragment into the DSB. Blunt-end double-stranded DNA fragments can be designed with 1-10 bp of microhomology, on both the 5′ and 3′ ends of the DNA fragment, that correspond to the 5′ and 3′ flanking sequence at the cut site of the protospacer in the genomic target site.

### Use of Custom CRISPRs in Molecular Breeding

In some embodiments, genome knowledge is utilized for targeted genetic alteration of a genome. At least one sgRNA can be designed to target at least one region of a genome to disrupt that region from the genome. This aspect of the disclosure may be especially useful for genetic alterations. The resulting plant could have a modified phenotype or other property depending on the gene or genes that have been altered. Previously characterized mutant alleles or introduced transgenes can be targeted for CRISPR-mediated modification, enabling creation of improved mutants or transgenic lines.

In another embodiment, a gene targeted for deletion or disruption may be a transgene that was previously introduced into the target plant or cell. This has the advantage of allowing an improved version of a transgene to be introduced or by allowing disruption of a selectable marker encoding sequence. In yet another embodiment, a gene targeted for disruption via CRISPR is at least one transgene that was introduced on the same vector or expression cassette as (an)other transgene(s) of interest, and resides at the same locus as another transgene. It is understood by those skilled in the art that this type of CRISPR-mediated modification may result in deletion or insertion of additional sequences. Thus it may, in certain embodiments, be preferable to generate a plurality of plants or cells in which a deletion has occurred, and to screen such plants or cells using standard techniques to identify specific plants or cells that have minimal alterations in their genomes following CRISPR-mediated modification. Such screens may utilize genotypic and/or phenotypic information. In such embodiments, a specific transgene may be disrupted while leaving the remaining transgene(s) intact. This avoids having to create a new transgenic line containing the desired transgenes without the undesired transgene.

In another aspect, the present disclosure includes methods for inserting a DNA fragment of interest into a specific site of a plant's genome, wherein the DNA fragment of interest is from the genome of the plant or is heterologous with respect to the plant. This disclosure allows one to select or target a particular region of the genome for nucleic acid (i.e., transgene) stacking (i.e., mega-locus). A targeted region of the genome may thus display linkage of at least one transgene to a haplotype of interest associated with at least one phenotypic trait, and may also result in the development of a linkage block to facilitate transgene stacking and transgenic trait integration, and/or development of a linkage block while also allowing for conventional trait integration.

### Use of Custom CRISPRs in Trait Integration

Directed insertion, in at least one genomic protospacer site, of DNA fragments of interest, via CRISPR-mediated cleavage allows for targeted integration of multiple nucleic acids of interest (i.e., a trait stack) to be added to the genome of a plant in either the same site or different sites. Sites for targeted integration can be selected based on knowledge of the underlying breeding value, transgene performance in that location, underlying recombination rate in that location, existing transgenes in that linkage block, or other factors. Once the stacked plant is assembled, it can be used as a trait donor for crosses to germplasm being advanced in a breeding pipeline or be directly advanced in the breeding pipeline.

The present disclosure includes methods for inserting at least one nucleic acid of interest into at least one site, wherein the nucleic acid of interest is from the genome of a plant, such as a QTL or allele, or is transgenic in origin. A targeted region of the genome may thus display linkage of at least one transgene to a haplotype of interest associated with at least one phenotypic trait (as described in U.S. Patent Application Publication No. 2006/0282911), development of a linkage block to facilitate transgene stacking and transgenic trait integration, development of a linkage block to facilitate QTL or haplotype stacking and conventional trait integration, and so on.

In another embodiment of this disclosure, multiple unique sgRNAs can be used to modify multiple alleles at specific loci within one linkage block contained on one chromosome by making use of knowledge of genomic sequence information and the ability to design custom sgRNAs as described in the art. A sgRNA that is specific for, or can be directed to, a genomic target site that is upstream of the locus containing the non-target allele is designed or engineered as necessary. A second sgRNA that is specific for, or can be directed to, a genomic target site that is downstream of the target locus containing the non-target allele is also designed or engineered. The sgRNAs may be designed such that they complement genomic regions where there is no homology to the non-target locus containing the target allele. Both sgRNAs may be introduced into a cell using one of the methods described above.

The ability to execute targeted integration relies on the action of the sgRNA:Cas-protein complex and the endonuclease activity of the Cas-associated gene product. This advantage provides methods for engineering plants of interest, including a plant or cell, comprising at least one genomic modification.

A custom sgRNA can be utilized in a CRISPR system to generate at least one trait donor to create a custom genomic modification event that is then crossed into at least one second plant of interest, including a plant, wherein CRISPR delivery can be coupled with the sgRNA of interest to be used for genome editing. In other aspects one or more plants of interest are directly transformed with the CRISPR system and at least one double-stranded DNA fragment of interest for directed insertion. It is recognized that this method may be executed in various cell, tissue, and developmental types, including gametes of plants. It is further anticipated that one or more of the elements described herein may be combined with use of promoters specific to particular cells, tissues, plant parts and/or developmental stages, such as a meiosis-specific promoter.

In addition, the disclosure contemplates the targeting of a transgenic element already existing within a genome for deletion or disruption. This allows, for instance, an improved version of a transgene to be introduced, or allows selectable marker removal. In yet another embodiment, a gene targeted for disruption via CRISPR-mediated cleavage is at least one transgene that was introduced on the same vector or expression cassette as (an)other transgene(s) of interest, and resides at the same locus as another transgene.

In one aspect, the disclosure thus provides a method for modifying a locus of interest in a cell comprising (a) identifying at least one locus of interest within a DNA sequence; (b) creating a modified nucleotide sequence, in or proximal to the locus of interest, that includes a protospacer sequence within a genomic target site for a first sgRNA according to the disclosure; (c) introducing into at least one cell the sgRNA and Cas-associated gene, wherein the sgRNA and/or Cas-associated gene is expressed transiently or stably; (d) assaying the cell for a CRISPR-mediated modification in the DNA making up or flanking the locus of interest; and (e) identifying the cell or a progeny cell thereof as comprising a modification in said locus of interest.

Another aspect provides a method for modifying multiple loci of interest in a cell comprising (a) identifying multiple loci of interest within a genome; (b) identifying multiple genomic protospacer sites within each locus of interest; (c) introducing into at least one cell multiple sgRNA and at least one Cas-associated gene according to the disclosure, wherein the cell comprises the genomic protospacer sites and the sgRNA and Cas-associated gene is expressed transiently or stably and creates a modified locus, or loci, that includes at least one CRISPR-mediated cleavage event; (d) assaying the cell for CRISPR-mediated modifications in the DNA making up or flanking each locus of interest; and (e) identifying a cell or a progeny cell thereof which comprises a modified nucleotide sequence at said loci of interest.

The disclosure further contemplates sequential modification of a locus of interest, by two or more sgRNAs and Cas-associated gene(s) according to the disclosure. Genes or other sequences added by the action of such a first CRISPR-mediated genomic modification may be retained, further modified, or removed by the action of a second CRISPR-mediated genomic modification.

The present invention thus includes a method for modifying a locus of interest in a crop plant such as maize (corn; Zea mays), soybean (Glycine max), cotton (Gossypium hirsutum; Gossypium sp.), peanut (Arachis hypogaea), barley (Hordeum vulgare); oats (Avena sativa); orchard grass (Dactylis glomerata); rice (Oryza sativa, including indica and japonica varieties); sorghum (Sorghum bicolor); sugar cane (Saccharum sp.); tall fescue (Festuca arundinacea); turfgrass species (e.g. species: Agrostis stolonfera, Poa pratensis, Stenotaphrum secundatum); wheat (Triticum aestivum); alfalfa (Medicago sativa); members of the genus Brassica, including broccoli, cabbage, carrot, cauliflower, Chinese cabbage; cucumber, dry bean, eggplant, tobacco, fennel, garden beans, gourd, leek, lettuce, melon, okra, onion, pea, pepper, pumpkin, radish, spinach, squash, sweet corn, tomato, watermelon, ornamental plants, and other fruit, vegetable, tuber, oilseed, and root crops, wherein oilseed crops include soybean, canola, oil seed rape, oil palm, sunflower, olive, corn, cottonseed, peanut, flaxseed, safflower, and coconut.

The genome modification may comprise a modified linkage block, the linking of two or more QTLs, disrupting linkage of two or more QTLs, gene insertion, gene replacement, gene conversion, deleting or disrupting a gene, transgenic event selection, transgenic trait donor selection, transgene replacement, or targeted insertion of at least one nucleic acid of interest.

### Definitions

The definitions and methods provided define the present disclosure and guide those of ordinary skill in the art in the practice of the present disclosure. Unless otherwise noted, terms are to be understood according to conventional usage by those of ordinary skill in the relevant art. Definitions of common terms in molecular biology may also be found in Alberts et al., Molecular Biology of The Cell, 5th Edition, Garland Science Publishing, Inc.: New York, 2007; Rieger et al., Glossary of Genetics: Classical and Molecular, 5th edition, Springer-Verlag: New York, 1991; King et al, A Dictionary of Genetics, 6th ed., Oxford University Press: New York, 2247; and Lewin, Genes IX, Oxford University Press: New York, 2007. The nomenclature for DNA bases as set forth at 37 CFR § 1.822 is used.

As used herein, “CRISPR-associated genes” refers to nucleic acid sequences that encode polypeptide components of clustered regularly interspersed short palindromic repeats (CRISPR)-associated systems (Cas). Examples include, but are not limited to, Cas3 and Cas9, which encode endonucleases from the CRISPR type I and type II systems, respectively.

As used herein, “single-guide RNA (sgRNA)” refers to a crRNA:tracrRNA fused hybrid single-stranded RNA molecule encoded by a customizable DNA element that, generally, comprises a copy of a spacer sequence which is complementary to the protospacer sequence of the genomic target site, and a binding domain for an associated-Cas endonuclease of the CRISPR complex.

As used herein, “genomic target site” refers to a protospacer and a protospacer adjacent motif (PAM) located in a host genome selected for targeted mutation and/or double-strand break.

As used herein, “protospacer” refers to a short DNA sequence (12 to 40 bp) that can be targeted for mutation, and/or double-strand break, mediated by enzymatic cleavage with a CRISPR system endonuclease guided by complementary base-pairing with the spacer sequence in the crRNA or sgRNA.

As used herein, “protospacer adjacent motif (PAM)” includes a 3 to 8 bp sequence immediately adjacent to the protospacer sequence in the genomic target site.

As used herein, “microhomology” refers to the presence of the same short sequence (1 to 10 bp) of bases in different polynucleotide molecules.

As used herein, “codon-optimized” refers to a polynucleotide sequence that has been modified to exploit the codon usage bias of a particular plant. The modified polynucleotide sequence still encodes the same, or substantially similar polypeptide as the original sequence but uses codon nucleotide triplets that are found in greater frequency in a particular plant.

As used herein, “non-protein-coding RNA (npcRNA)” refers to a non-coding RNA (ncRNA) which is a precursor small non-protein coding RNA, or a fully processed non-protein coding RNA, which are functional RNA molecules that are not translated into a protein.

As used herein, the term “chimeric” refers to the product of the fusion of portions of two or more different polynucleotide molecules, or to a gene expression element produced through the manipulation of known elements or other polynucleotide molecules. Novel chimeric regulatory elements can be designed or engineered by a number of methods. In one embodiment of the present disclosure, a chimeric promoter may be produced by fusing the 5′ portion of a U6 promoter from corn chromosome 1, which includes at least one Monocot-Specific Promoter (MSP) element, to the 3′ portion of the U6 promoter from corn chromosome 8, which includes an Upstream Sequence Element (USE) and a TATA Box. The resultant chimeric promoter may have novel expression properties relative to the first or second promoters.

As used herein, “promoter” refers to a nucleic acid sequence located upstream or 5′ to a translational start codon of an open reading frame (or protein-coding region) of a gene and that is involved in recognition and binding of RNA polymerase I, II, or III and other proteins (trans-acting transcription factors) to initiate transcription. A “plant promoter” is a native or non-native promoter that is functional in plant cells. Constitutive promoters are functional in most or all tissues of a plant throughout plant development. Tissue-, organ- or cell-specific promoters are expressed only or predominantly in a particular tissue, organ, or cell type, respectively. Rather than being expressed “specifically” in a given tissue, plant part, or cell type, a promoter may display “enhanced” expression, i.e., a higher level of expression, in one cell type, tissue, or plant part of the plant compared to other parts of the plant. Temporally regulated promoters are functional only or predominantly during certain periods of plant development or at certain times of day, as in the case of genes associated with circadian rhythm, for example. Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

As used herein, an “expression cassette” refers to a polynucleotide sequence comprising at least a first polynucleotide sequence capable of initiating transcription of an operably linked second polynucleotide sequence and optionally a transcription termination sequence operably linked to the second polynucleotide sequence.

A palindromic sequence is a nucleic acid sequence that is the same whether read 5′ to 3′ on one strand or 3′ to 5′ on the complementary strand with which it forms a double helix. A nucleotide sequence is said to be a palindrome if it is equal to its reverse complement. A palindromic sequence can form a hairpin.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following examples are included to demonstrate embodiments of the disclosure. It should be appreciated by those of skill in the art that many changes can be made in the specific embodiments which are disclosed and still obtain a like or similar result without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### Example 1

### Identification of Promoters to Express sgRNA

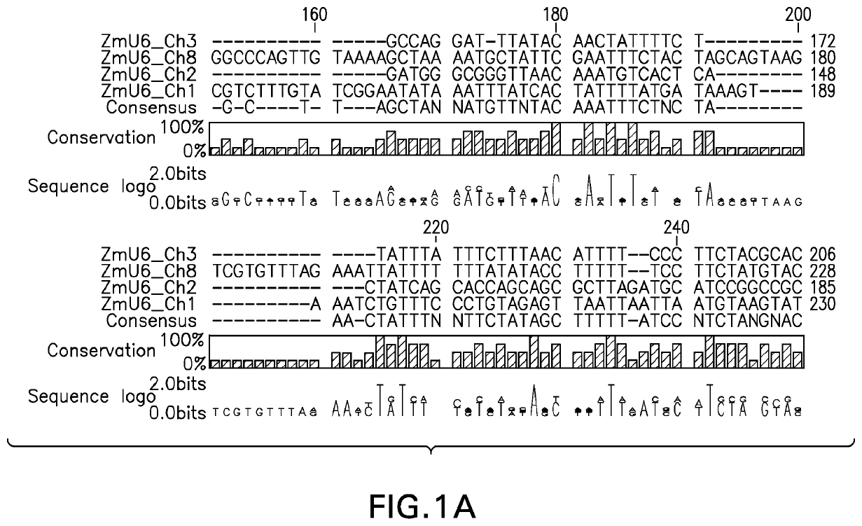

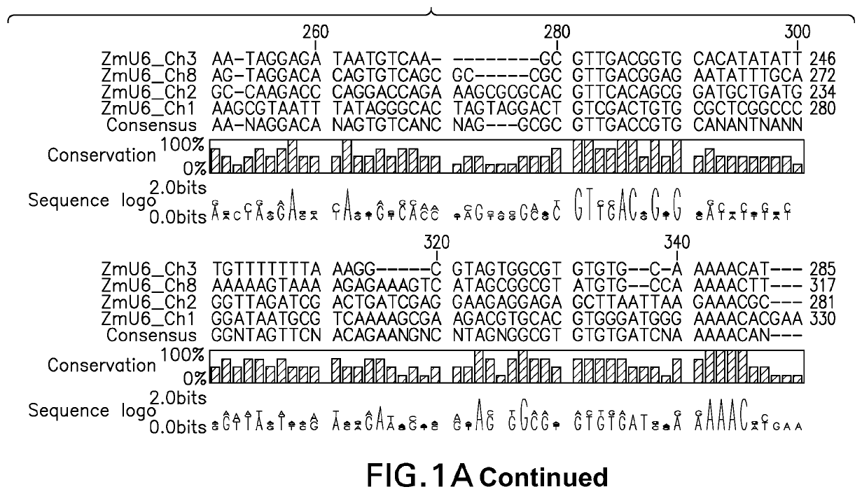

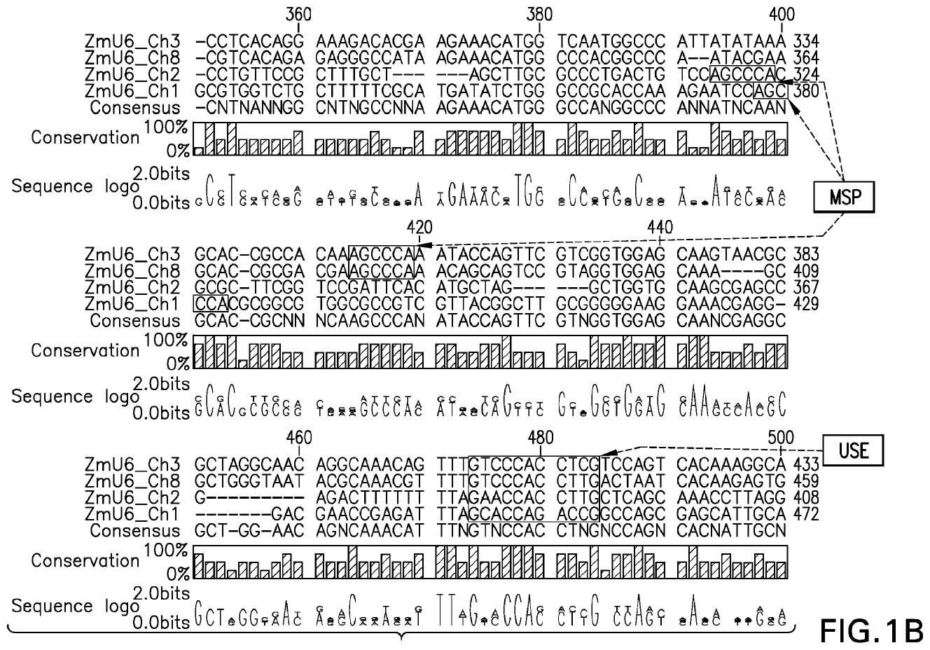

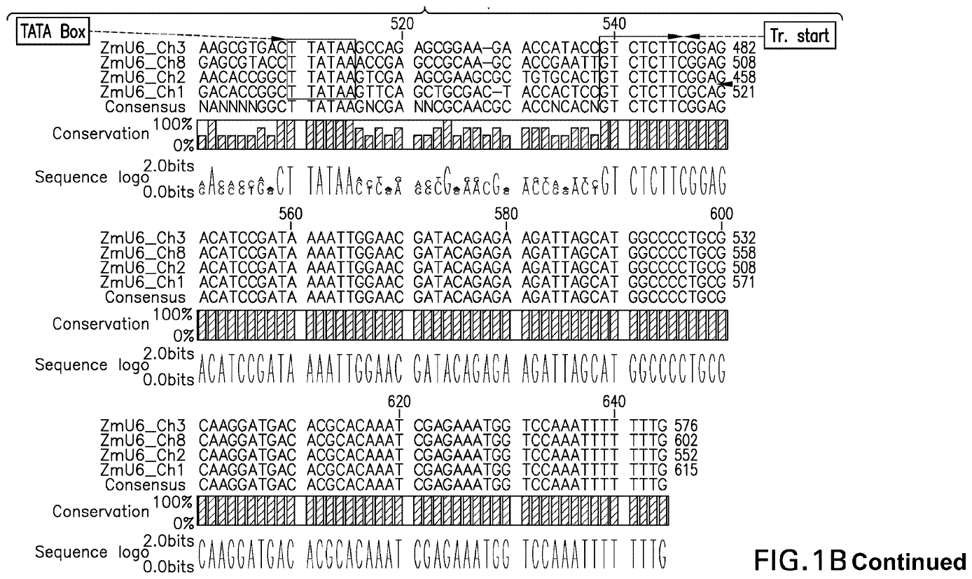

To enable genome engineering in corn, soy, and tomato using the CRISPR-based gene targeting system, novel U6 promoters native to these three genomes were identified. After BLAST searching for the highly conserved U6 gene in corn, soy, and tomato genomes, 200-600 bp of sequence upstream of these putative U6 genes was selected to test for promoter function (Table 1). Four U6 promoters were identified from the corn B73 genome, one each on chromosome 1 (SEQ ID NO:1), chromosome 2 (SEQ ID NO:3), chromosome 3 (SEQ ID NO:5), and chromosome 8 (SEQ ID NO:7). A multiple sequence alignment of these four corn U6 promoters and corresponding U6 genes was compiled as shown in FIGS. 1A and B. For each of these corn U6 promoters, conserved U6 promoter motifs (e.g., TATA Box, Upstream Sequence Element (USE), and Monocot-Specific Promoter (MSP) elements (Connelly, Mol. Cell Biol. 14:5910-5919, 1994) are present (FIG. 1B). A guanine nucleobase following the poly-T tracts was conserved among these four genes, and may have a significant role in transcription. The sequence consensus, and percent conservation are presented below the alignment (FIG. 1). Based on the multiple sequence alignment, the conserved motifs of these U6 promoters were within the 140 bp proximal to the transcription start site. Based on the proximity of these conserved U6 promoter motifs, 200 bp of the proximal upstream sequence from the transcription start site for each of the corn chromosome U6 promoters, chromosome 1 (SEQ ID NO:2), chromosome 2 (SEQ ID NO:4), chromosome 3 (SEQ ID NO:6), and chromosome 8 (SEQ ID NO:8) was selected for testing for efficient promoter activity in sgRNA expression cassettes.

In addition to the four corn U6 promoters, chimeric U6 promoters were designed. Four chimeric corn U6 promoters were designed using differing combinations of the corn U6 promoters from chromosome 1, 2, and 8, with each chimeric promoter being 397 bp in length. The breakpoints of the chimeras were determined so that the conserved elements (e.g., USE, MSP, and TATA box) of different chromosomal origins were mixed in the new chimeric U6 promoters but retained their relative spacing to the native corn U6 promoters. For example, the 5′ end of the U6 promoter including MSP and USE were derived from one chromosome, while the 3′ end including the TATA box and one or more MSP elements were derived from a second chromosome. Although the corn U6 promoter from chromosome 2 was not a very strong promoter in its native form, it included more than one MSP element. Consequently, chimeras that include mainly chromosome 1 and/or 8 sequence can also include one or more chromosome 2 MSP elements. Specifically, the 5′ portion of chimera 1 (SEQ ID NO:17) is derived from the U6 promoter from corn chromosome 1 (SEQ ID NO:1), including one MSP element, and the 3′ portion of this chimera is derived from the U6 promoter from corn chromosome 8 (SEQ ID NO:7), including a USE element and a TATA box. Similarly, the 5′ portion of chimera 2 (SEQ ID NO:18) is derived from the U6 promoter from corn chromosome 1 (SEQ ID NO:1), including one MSP element, and the 3′ portion of this chimera is derived from the U6 promoter from corn chromosome 8 (SEQ ID NO:7), including a second MSP element, a USE element, and a TATA box. The 5′ portion of chimera 3 (SEQ ID NO:19) is derived from the U6 promoter from corn chromosome 8 (SEQ ID NO:7), including one MSP element, and the 3′ portion of this chimera is derived from the U6 promoter from corn chromosome 1 (SEQ ID NO:1), including a second MSP element, a USE element, and a TATA box. Additionally, for chimera 3, there is a 3 bp deletion beginning at bp 100 of SEQ ID NO:7, and the 5′ end of the chimera begins with 5′-AAG-3′. Chimera 4 (SEQ ID NO:20) was derived from the U6 promoter from corn chromosome 8 (SEQ ID NO:7), including the MSP element, the USE element and the TATA box. However, this chimera also includes two additional MSP elements (for a total of 3 MSP elements) derived from the U6 promoter of corn chromosomes 1 and 2.

TABLE 1
U6 promoters from corn (Zea mays), tomato (Solanum lycopersicum),
and soybean (Glycine max), their chromosomal source and length.

| SEQ ID NO. | Source | Chromosome | Length (bp) |
|---|---|---|---|
| 1 | Zea mays | 1 | 397 |
| 2 | Zea mays | 1 | 200 |
| 3 | Zea mays | 2 | 397 |
| 4 | Zea mays | 2 | 200 |
| 5 | Zea mays | 3 | 397 |
| 6 | Zea mays | 3 | 200 |
| 7 | Zea mays | 8 | 397 |
| 8 | Zea mays | 8 | 200 |
| 9 | Solanum lycopersicum | 10 | 540 |
| 10 | Solanum lycopersicum | 1 | 600 |
| 11 | Solanum lycopersicum | 7 | 540 |
| 12 | Glycine max | 6 | 540 |
| 13 | Glycine max | 16 | 540 |
| 14 | Glycine max | 19 | 540 |
| 15 | Glycine max | 4 | 540 |
| 16 | Glycine max | 19 | 420 |
| 17 | Zea mays | Chimeric: 1 + 8 | 397 |
| 18 | Zea mays | Chimeric: 1 + 8 | 397 |
| 19 | Zea mays | Chimeric: 8 + 1 | 397 |
| 20 | Zea mays | Chimeric: 8 + 2 + 1 + 8 | 397 |

### Example 2

### Identification of Cas9 Genes to Enable Genome Engineering in Plants

The S. pyogenes Cas9 sequence (SEQ ID NO:28 is the polypeptide sequence of Cas9 with NLS, and SEQ ID NO:96 is the polypeptide sequence of Cas9 without NLS) was used for CRISPR-mediated site-directed targeting of a reporter construct in immature corn embryos. For expression, the codon-optimized nucleotide sequence of Cas9 was designed into an expression vector capable of expression in a plant. This Cas9 expression vector contained a 35S promoter driving expression of the Cas9 open reading frame, a NLS sequence incorporated into the 3′ end of the Cas9 coding region, and a Nos transcription termination sequence (SEQ ID NO:29).

A Cas9 protein (SEQ ID NO:26), and a monocot codon-optimized version of the nucleotide sequence encoding the same (SEQ ID NO:27), were identified from the plant-related bacteria Bradyrhizobium, and can be useful for increasing the robustness of CRISPR/Cas-mediated genome modification in plants. A Cas9 protein (SEQ ID NO:69) and a monocot codon-optimized version thereof (SEQ ID NO:68), were identified from Streptococcus thermophilus, and can be useful for increasing the robustness of CRISPR/Cas-mediated genome modification in plants. Additional Cas9 genes from plant-related bacteria (e.g., symbiotic or pathogenic bacteria) can also be identified.

### Example 3

### Single-Guide RNA Cassette Design

A set of single-guide RNA (sgRNA) expression cassettes were designed to target a protospacer in a corn genomic target site referred to as Zm7 (5′-GCCGGCCAGCATTTGAAACATGG-3′, SEQ ID NO:22). The different expression cassettes included one of the 397 bp U6 promoters from corn: chromosome 1 (SEQ ID NO:30), chromosome 2 (SEQ ID NO:32), chromosome 3 (SEQ ID NO:34), or chromosome 8 (SEQ ID NO:36); or one of the 200 bp U6 promoter from corn: chromosome 1 (SEQ ID NO:31), chromosome 2 (SEQ ID NO:33), chromosome 3 (SEQ ID NO:35), or chromosome 8 (SEQ ID NO:37). Each expression cassette also contained, i) the U6 poly-T terminator conserved in each of the four corn U6 genes; ii) a sgRNA including a copy of the spacer sequence 5′-GCCGGCCAGCATTTGAAACA-3′ (SEQ ID NO:23) corresponding to the protospacer of the Zm7 genomic target site (SEQ ID NO:22); and iii) the conserved 3′ domain of a sgRNA providing the Cas endonuclease binding domain, and ending with the U6 poly-T tract (SEQ ID NO:21).

Similarly, a set of sgRNA cassettes were designed with one of the four corn U6 397 bp promoters (SEQ ID NO:30, SEQ ID NO:32, SEQ ID NO:34, or SEQ ID NO:36; see Table 2), and the spacer sequence of the sgRNA complementary to the protospacer of the corn genomic target site referred to as Zm231 (SEQ ID NO:24). Table 3 lists the corresponding SEQ ID NOs for the DNA and RNA sequences of the sgRNAs containing the Zm7, Zm231, and Zm14 target sites. A negative control sgRNA cassette was designed with the corn U6 397 bp promoter from corn chromosome 8 (SEQ ID NO:36) and spacer sequence of the sgRNA complementary to the protospacer of the corn genomic target site referred to as Zm14 (SEQ ID NO:24). This negative control sgRNA cassette was designed with a spacer sequence of the sgRNA that is non-complementary to the protospacer sequence of the Zm231 corn genomic target site. Inclusion of a sgRNA comprising the spacer sequence complementary to the Zm4 corn genomic target site will not result in CRISPR/Cas-mediated cleavage of the protospacer sequence of the Zm231 corn target protospacer site. These Zm231 and Zm24 sgRNA cassettes are represented by SEQ ID NO:38, SEQ ID NO:39, SEQ ID NO:40, SEQ ID NO:41, and SEQ ID NO:42 (Table 2). Each of these sgRNA cassettes also contains at the 3′ end of the sgRNA sequence a U6 poly-T tract.

TABLE 2
Cassettes with the indicated corn (Zea mays)
U6 promoters and sgRNA containing spacers
complementary to the protospacer sequence
of the indicated corn genomic target sites.

| | U6 Promoter | | |
|---|---|---|---|
| SEQ ID | from | U6 Promoter | Genomic |
| NO. | Chromosome | Length (bp) | target site |
| 30 | 1 | 397 | Zm7 |
| 31 | 1 | 200 | Zm7 |
| 32 | 2 | 397 | Zm7 |
| 33 | 2 | 200 | Zm7 |
| 34 | 3 | 397 | Zm7 |
| 35 | 3 | 200 | Zm7 |
| 36 | 8 | 397 | Zm7 |
| 37 | 8 | 200 | Zm7 |
| 38 | 1 | 397 | Zm231 |
| 39 | 2 | 397 | Zm231 |
| 40 | 3 | 397 | Zm231 |
| 41 | 8 | 397 | Zm231 |
| 42 | 8 | 397 | Zm14 |

TABLE 3
DNA and RNA sequences of Streptococcus
pyogenes sgRNAs containing spacer sequences
complementary to the protospacer sequence of the
corn genomic target sites Zm7, Zm231, and Zm14.

| SEQ ID NO. | | |
|---|---|---|
| DNA | RNA | Genomic target site |
| 76 | 79 | Zm7 |
| 77 | 80 | Zm231 |
| 78 | 81 | Zm14 |

### Example 4

### CRISPR Activity in Corn—Modified GUS Reporter Assay

To determine the activity of CRISPR/Cas-mediated gene-targeting efficiency in corn, a system for the transient expression of a reporter gene in immature corn embryos was used. In addition to the sgRNA cassettes described above, the design incorporated an expression cassette containing the Cas9 endonuclease of Streptococcus pyogenes (SEQ ID NO:28) containing a nuclear localization signal (NLS) sequence and was codon-optimized for expression in corn.

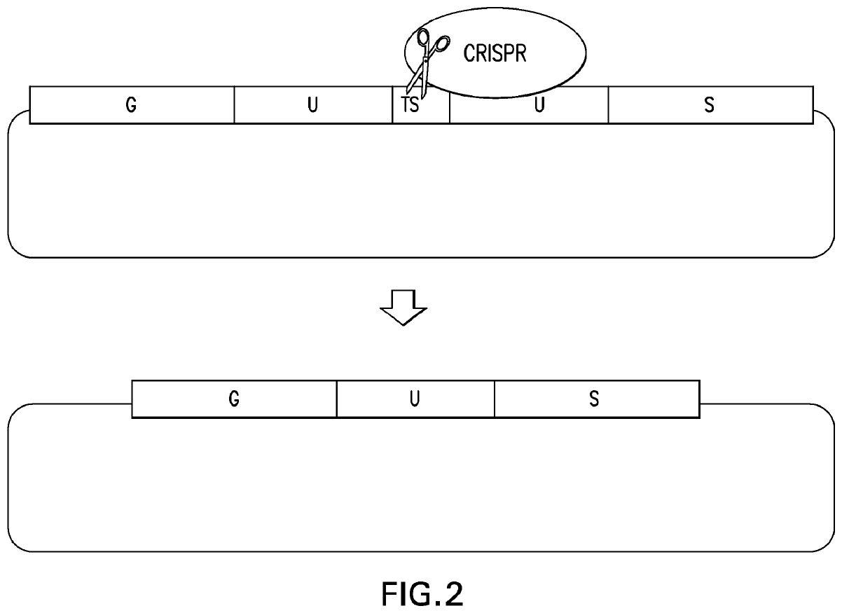

The reporter gene construct for these experiments was a cassette containing a modified 0-glucuronidase (GUS) coding sequence with a corn genomic target site (protospacer and PAM) for targeted CRISPR cleavage (e.g., the Zm7 (SEQ ID NO:22), Zm231 (SEQ ID NO:44), or Zm14 (SEQ ID NO:43)) engineered into the reporter gene and surrounded by an internal direct repeat of the GUS coding sequence (FIG. 2). When co-delivered with expression vectors for CRISPR components, if the CRISPR system cleaves the protospacer sequence, the endogenous plant single-strand annealing (SSA) pathway of homologous recombination DNA repair will reconstitute a functional GUS gene. These modified GUS reporter constructs were named GU-Zm7-US, GU-Zm231-US, or GU-Zm14-US, referring to the corn genomic target site inserted into the GUS gene, Zm7, Zm231, and Zm14, respectively. One of the modified GUS reporter gene cassettes was co-delivered with expression vectors for the other CRISPR components (e.g., one of the sgRNA cassettes) and the expression cassette encoding the Cas9 endonuclease (SEQ ID NO:28). Expression cassettes were mixed and co-coated on 0.6 μM gold particles using standard protocols. 3-day old pre-cultured immature corn embryos were then bombarded with these prepared gold particles. Embryos were maintained in culture for 3-5 days after bombardment and then processed for histochemical staining using X-Gluc (5-bromo-4-chloro-3-indolyl glucuronide) and standard laboratory protocols.

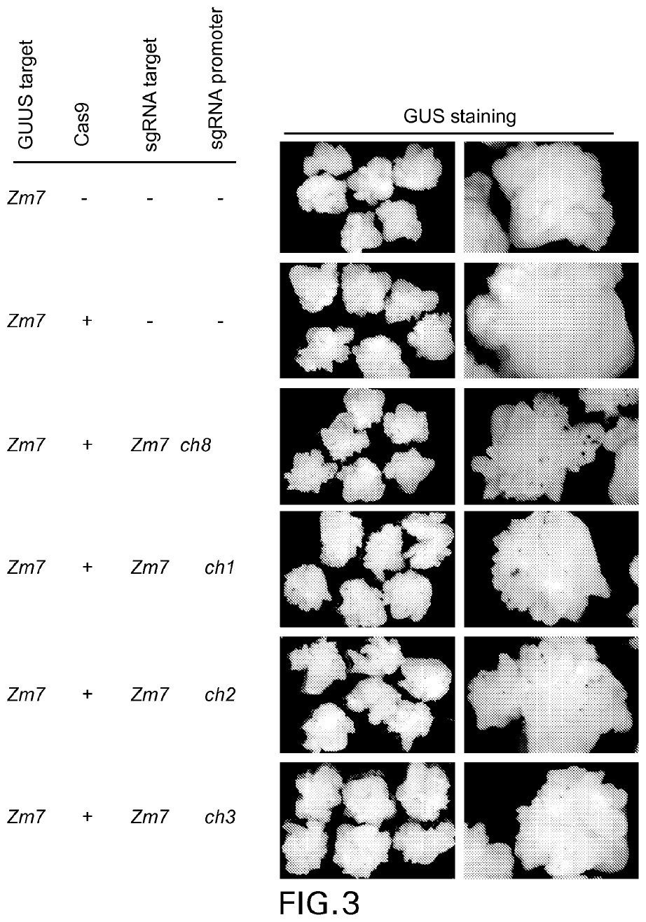

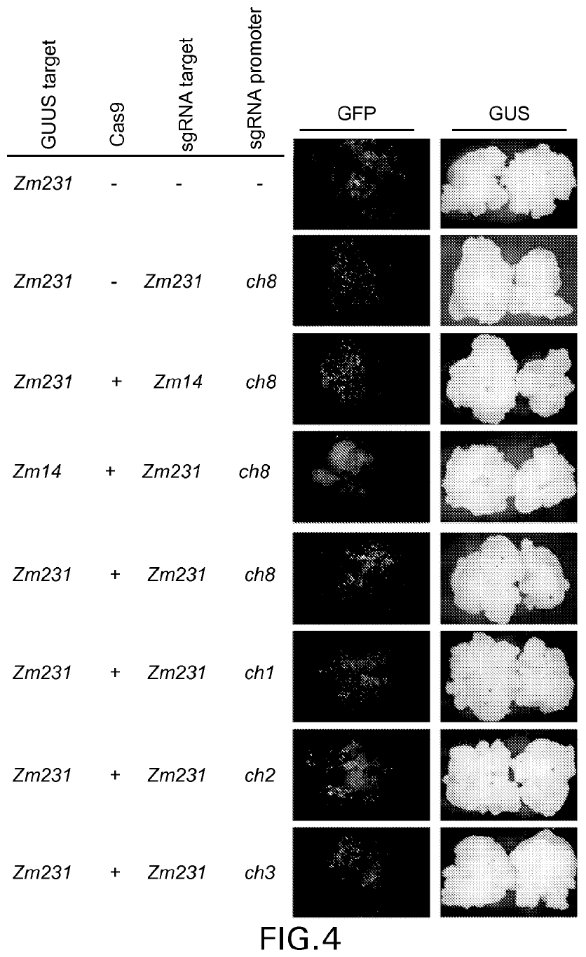

If CRISPR-mediated Cas9 endonuclease activity occurs at the protospacer site in the modified reporter gene construct, then GUS activity is detected as blue foci using histochemical staining and X-Gluc (FIGS. 3 and 4).

Separate expression cassettes were designed to contain one of four corn U6 promoters (from chromosomes 1, 2, 3, and 8) driving expression of a sgRNA containing a spacer sequence complementary to the protospacer of the corn Zm7 genomic target site (FIG. 3). To prepare samples for the expression assay, 0.6 M gold particles were coated with 0.6 pmol of one of the Zm7-sgRNA constructs and 0.3 pmol of each of the other constructs (Cas9 expression cassette and the Zm7-modified reporter construct (GU-Zm7-US)). Once the coated gold particles were prepared, ¼ of the mixture was used for bombardment of 3-day old immature corn embryos using standard protocols. More than 50 immature corn calli were bombarded for each set of constructs evaluated, and staining was done 5 days post-bombardment. Following staining, photographs of representative calli (overview of several calli and a close-up view of a single callus) were taken (FIG. 3). The modified reporter construct GU-Zm7-US was designed to contain the Zm7 genomic target site (SEQ ID NO:22), and the sgRNA was designed to contain a copy of the Zm7 spacer (SEQ ID NO:23). The Zm7-sgRNA spacer was incorporated into expression cassettes with one of the four 397 bp corn U6 promoters from chromosome 1 (SEQ ID NO:30), chromosome 2 (SEQ ID NO:32), chromosome 3 (SEQ ID NO:34), or chromosome 8 (SEQ ID NO:36). Negative controls used in the transformation included the modified reporter construct GU-Zm7-US with the Zm7 genomic target site and: (1) lacking both the Cas9 endonuclease expression cassette and the Zm7-sgRNA expression cassette; or (2) lacking just the Zm7-sgRNA expression cassette (FIG. 3). For both of these controls no blue sectors were detected, indicating no CRISPR-mediated cleavage of the modified reporter construct had occurred. The results from evaluation of the four different 397 bp corn U6 promoters in driving expression of the Zm7-sgRNA cassette showed that while all four 397 bp corn U6 promoters worked (i.e., blue sectors detected in the calli), the efficacy of the different promoters varied (as evidenced by the size and number of blue sectors in the calli). The U6 promoter from corn chromosome 8 showed the most efficacy, followed by the U6 promoter from chromosome 1. The U6 promoters from chromosomes 2 and 3 showed similar efficacy to each other (Chr 8>Chr 1>Chr2≈Chr3).

The specificity of the CRISPR/Cas9 system in this corn expression system was evaluated by testing mismatches between the protospacer sequence within the genomic target site in the modified GUUS reporter gene construct and the spacer sequence included in the varying sgRNA constructs (FIG. 4). As in the experiment described above, 0.6 M gold particles were coated with one or more constructs; 0.3 pmol of the individual modified GUUS reporter construct (GUUS target), 0.16 pmol of the Cas9 endonuclease expression cassette, 0.3 pmol of the individual sgRNA cassettes, and 0.03 pmol of a transformation control construct expressing green fluorescent protein (GFP) (FIG. 4). Once the coated gold particles were prepared, ¼ of the mixture was used for bombardment of 3-day old immature corn embryos using standard protocols. More than 50 immature corn calli were bombarded for each set of constructs evaluated. Tissue was maintained in culture for 3 days post-bombardment. Determination of GFP expression by fluorescence microscopy was done on day 1 and again on day 3 to validate uniform bombardment and transformation. After the fluorescence microscopy on day 3, the calli were processed for X-Gluc staining and fluorescent and light micrographs of representative calli were taken (FIG. 4). The fluorescent staining for all calli indicated good transformation.

Negative controls used in the transformation included the modified reporter construct GU-Zm231-US with the Zm231 genomic target site (1) lacking both the Cas9 endonuclease expression cassette and any sgRNA expression cassette; or (2) having a Zm231-sgRNA expression cassette with a corn U6 promoter from chromosome 8, but lacking the Cas9 endonuclease expression cassette (FIG. 4). Both of these controls showed no blue sectors detected with X-Gluc staining, indicating no CRISPR-mediated cleavage of the modified reporter construct had occurred (FIG. 4).

The specificity of the CRISPR/Cas9 system was also evaluated using controls including a mismatch between the protospacer site in the modified GUUS reporter construct and the sgRNA spacer sequence. Specifically, the mismatch was between the modified reporter construct GU-Zm231-US with the Zm231 genomic target site and (1) the sgRNA expression cassette with the Zm14 spacer and a corn U6 promoter from chromosome 8; or (2) the sgRNA expression cassette with the Zm231 spacer sequence and a corn U6 promoter from chromosome 8 (FIG. 4).

Finally, the 397 bp corn U6 promoters (chromosome 1, 2, 3, and 8) were each used to generate sgRNA expression cassettes with the Zm231 genomic target site. These were each co-transformed with the modified reporter construct GU-Zm231-US made with the Zm231 genomic target site. Results indicated that when the sgRNA spacer sequence and the genomic target site of the reporter construct were mismatched, there was very little GUS activity detected. By contrast, when the sgRNA spacer sequence and the genomic target site of the reporter construct were matched, many large blue foci were detected (FIG. 4). The U6 promoter from corn chromosome 8 may have higher efficacy (based on the assumption that efficacy correlates to blue foci which were more numerous, larger in size, and darker in staining intensity), followed by the U6 promoter from corn chromosome 1. The U6 promoters from corn chromosomes 2 and 3 showed similar efficacy to each other (Chr 8>Chr 1>Chr2≈Chr3).

The sgRNA driven by the U6 promoter from corn chromosome 8 consistently showed high activity. These findings suggest that different corn U6 promoters have differing activities, and further highlights the usefulness of the U6 promoter derived from corn chromosome 8 in the CRISPR/Cas system of targeted genome modification.

### Example 5

### Blunt-End Oligonucleotide Integration

The CRISPR/Cas9 system was evaluated for targeting efficacy of insertion of a blunt-end double-stranded DNA fragment into one of three genomic target sites, identified as Zm_L70a (SEQ ID NO:47), Zm_L70c (SEQ ID NO:59), and Zm_L70d (SEQ ID NO:61) within the corn genome. Each of these three genomic target sites is unique in the corn genome. If the CRISPR components are capable of endonuclease activity and introduce a double strand break (DSB) in the protospacer of the selected genomic target site, then the endogenous corn non-homologous end-joining DNA repair system will insert the blunt-end double-stranded DNA fragment into the DSB.

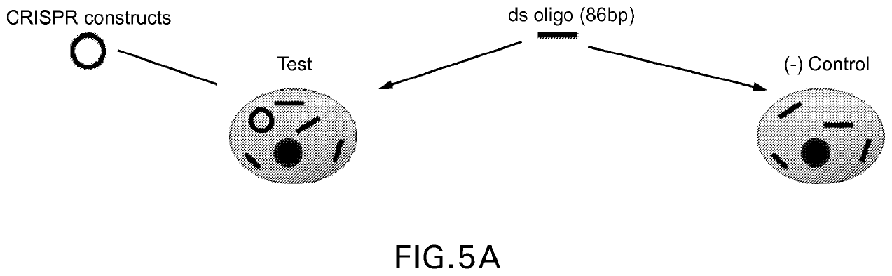

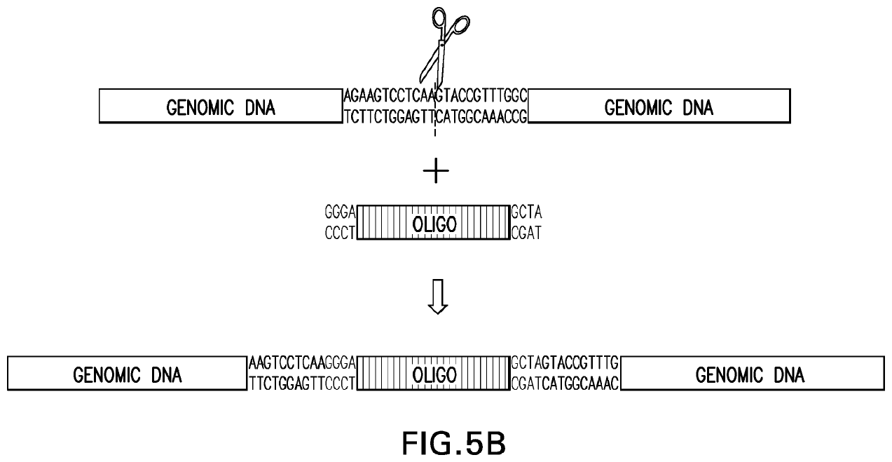

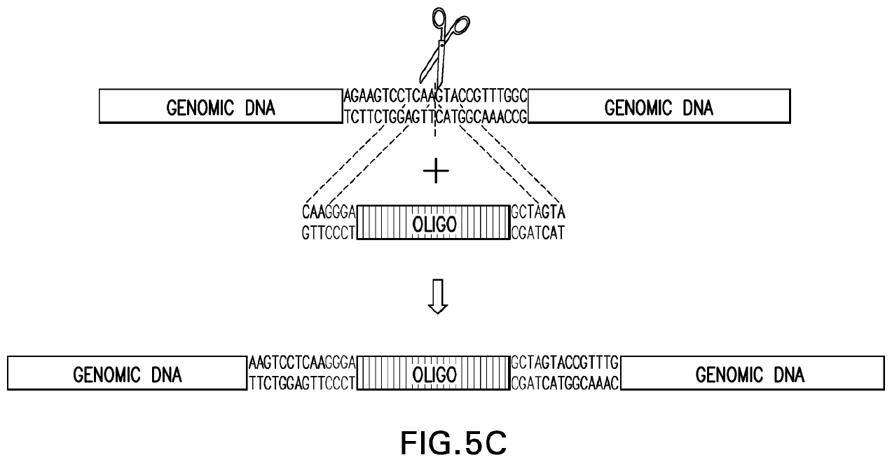

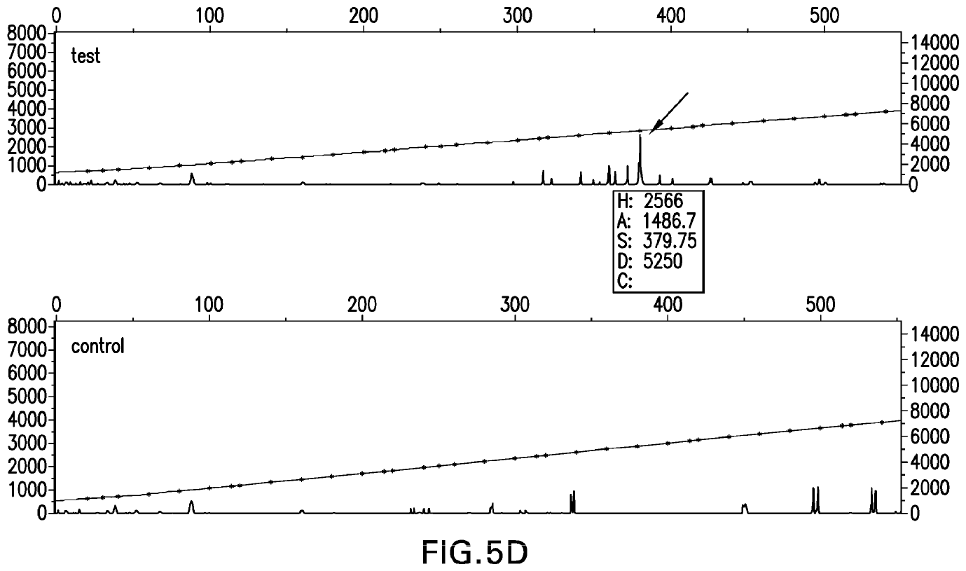

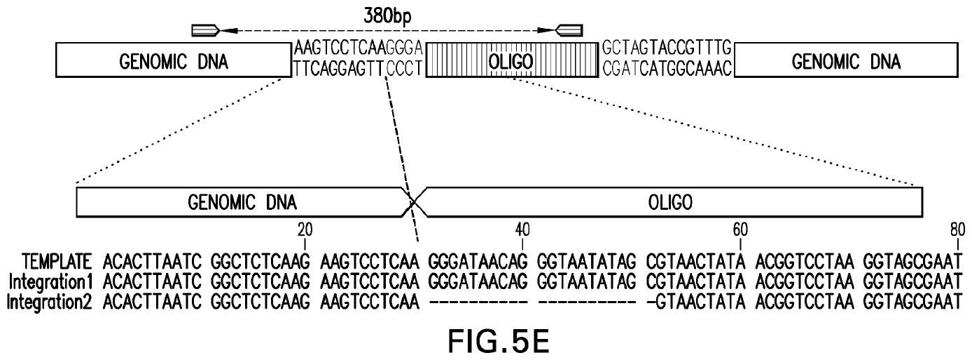

Complementary oligonucleotides were pre-annealed to form blunt-ended double-stranded DNA fragments, and these were co-transformed with CRISPR constructs into corn protoplasts (FIG. 5A). The oligonucleotide pairs were designed to either (1) not contain microhomology regions (see FIG. 5B), or (2) contain on each end (5′ and 3′) a 3 bp microhomology to the corresponding 5′ and 3′ flanking sequence at the cut site of the protospacer in the genomic target site (FIG. 5C). The microhomology sequences may promote blunt-end double-strand DNA fragment integrations through a mechanism of microhomology-driven non-homologous end-joining at the genomic target site. The two sequences of the oligonucleotide pair without microhomology sequence were SEQ ID NO:45 and SEQ ID NO:46. The three pairs of oligonucleotides, each containing microhomology to their respective genomic target site, were annealed in pairwise combinations of the following oligonucleotides: (1) SEQ ID NO:62 and SEQ ID NO:63 (microhomology to Zm_L70a); (2) SEQ ID NO:64 and SEQ ID NO:65 (microhomology to Zm_L70c); and (3) SEQ ID NO:66 and SEQ ID NO:67 (microhomology to Zm_L70d) to form blunt-end double-strand DNA fragments.

For these blunt-end double-strand DNA fragment integration assays, the CRISPR constructs used included the Cas9 endonuclease expression cassette described above, and one of three sgRNA expression cassettes. The three sgRNA expression cassettes were each driven by the 397 bp version of the U6 promoter from corn chromosome 8 (SEQ ID NO:7) and contained the spacer sequence corresponding to the genomic target sites: Zm_L70a (SEQ ID NO:48), Zm_L70c (SEQ ID NO:58), and Zm_L70d (SEQ ID NO:60). Differing combinations of the CRISPR components and oligonucleotides for these assays were mixed as follows: 0.6 pmol of the Cas9 expression cassette, 1.6 pmol of one of the sgRNA expression cassettes, and 35 pmol of the pre-annealed, oligonucleotide pair, and, using a standard PEG-mediated protocol, transformed into aliquots of corn leaf protoplast suspensions containing about 320,000 cells. Two days later, corn protoplasts were harvested and analyzed for insertion of the blunt-end double-strand DNA fragment into the particular L70 genomic target site targeted by the unique sgRNA selected in each case (Table 4). The negative control was the omission of the Cas9 expression cassette during the corn protoplast transformation.

To detect the insertion of the blunt-end double strand DNA fragment into the corn chromosome, DNA was extracted and high-throughput thermal amplification (PCR) was done with multiple pairs of primers (Table 5). As the blunt-end double strand DNA fragment may insert into the CRISPR cleaved chromosomal DNA in either orientation, primers were designed to one strand of the blunt-end double strand DNA fragment and to both flanking genomic regions, with each primer pair spanning the junction of the insertion site. The PCR amplicons were separated on a fragment analysis platform (ABI3730 DNA analyzer) from Life Technologies (Grand Island, NY). This platform, which is more sensitive than gel-based electrophoresis methods and has single-bp resolution, confirmed whether the amplicons originated from the template of interest and whether they were specific to the experimental treatment conditions.

TABLE 4
DNA and RNA sequences of Streptococcus
pyogenes sgRNA containing spacer sequences
complementary to the protospacer sequence of
the corn genomic target sites L70a, L70c, L70d.

| SEQ ID NO. | | |
|---|---|---|
| DNA | RNA | Genomic target site |
| 82 | 85 | Zm_L70a |
| 83 | 86 | Zm_L70c |
| 84 | 87 | Zm_L70d |

One representative fragment analysis profile is shown in FIG. 5D (Experiment T3, Table 5). Amplification of DNA extracted from corn protoplasts transformed with Cas9, sgRNA containing spacer sequences complementary to the protospacer sequence of the Zm_L70c corn genomic target site (SEQ ID NO:83), and the blunt-end double-stranded DNA fragment without microhomology, using primers at the Zm_L70c genomic target site (SEQ ID NO:49, primer specific for the inserted blunt-end double-strand DNA fragment, and SEQ ID NO:55, primer specific for flanking genomic DNA) revealed a major peak of the expected size and several additional peaks of similar sizes (arrow) (FIG. 5D, top panel). By contrast, no amplification products were seen from DNA extracted from the negative control transformations (FIG. 5D, bottom panel). This PCR profile was consistent with double-stranded breaks repaired erroneously by non-homologous end-joining, resulting in introduction of short indels at the site of repair.

To confirm that the blunt-end double-strand DNA fragment was incorporated at the genomic target site, the PCR amplicons were cloned and sequenced (Table 5). Negative controls lacking Cas9 proteins did not produce PCR products. Seven of the ten experiments showed the expected pattern: a positive PCR product of the expected size for the test samples, and no PCR product for control samples. The seven experiments showing a positive PCR product included experiments demonstrating integrations occurring for both blunt-end double-strand DNA fragments with and without microhomology. Experiments T1 and T7 failed to detect targeted integrations in either test or control samples. PCR products from six of the experiments were cloned and sequenced, confirming the expected DNA fragment-chromosome junctions for blunt-end double-strand DNA fragment integration. Sequencing results showed the presence of both full-length and truncated DNA fragments (indels) present at the site of blunt-end double-strand DNA fragment integration (see, e.g., FIG. 5E, Experiment T1). Sequences were consistent with the fragment analysis (FIG. 5D) and demonstrated that CRISPR/Cas9 can target native, sequence-specific, chromosomal loci for cleavage in corn protoplasts. These results also demonstrated successful blunt-end double-strand DNA fragment integration with and without regions of microhomology.

TABLE 5
Blunt-end oligonucleotide insertion assay.

| | | Genomic | | | | Expected | | |
|---|---|---|---|---|---|---|---|---|
| | | protospacer/ | Micro- | | Primer pairs | amplicon | Expected | Sequenced |
| Experiment | Treatments | target site | homology | Orientation | (SEQ ID NOs) | size (bp) | amplicon | amplicon |
| T1 | test | L70a | − | + | 50/49 | 408 | − | − |
| | (−) control | L70a | − | + | 50/49 | N/A | − | − |
| T2 | test | L70a | − | − | 51/49 | 324 | + | + |
| | (−) control | L70a | − | − | 51/49 | N/A | − | − |
| T3 | test | L70c | − | + | 55/49 | 384 | + | + |
| | (−) control | L70c | − | + | 55/49 | N/A | − | − |
| T4 | test | L70c | − | − | 54/49 | 411 | + | + |
| | (−) control | L70c | − | − | 54/49 | N/A | − | − |
| T5 | test | L70c | + | + | 55/49 | 384 | + | + |
| | (−) control | L70c | + | + | 55/49 | N/A | − | − |
| T6 | test | L70c | + | − | 54/49 | 411 | + | − |
| | (−) control | L70c | + | − | 54/49 | N/A | − | − |
| T7 | test | L70d | − | + | 56/49 | 359 | − | − |
| | (−) control | L70d | − | + | 56/49 | N/A | − | − |
| T8 | test | L70d | − | − | 57/49 | 356 | + | + |
| | (−) control | L70d | − | − | 57/49 | N/A | − | − |
| T9 | test | L70d | + | + | 56/49 | 359 | + | + |
| | (−) control | L70d | + | + | 56/49 | N/A | − | − |
| T10 | test | L70d | + | − | 57/49 | 356 | + | − |
| | (−) control | L70d | + | − | 57/49 | N/A | * | − |

Where * = sample contaminated.

### Example 6

### Targeted Genome Modification with CRISPR/Cas9 Complex Genes from Streptococcus thermophilus

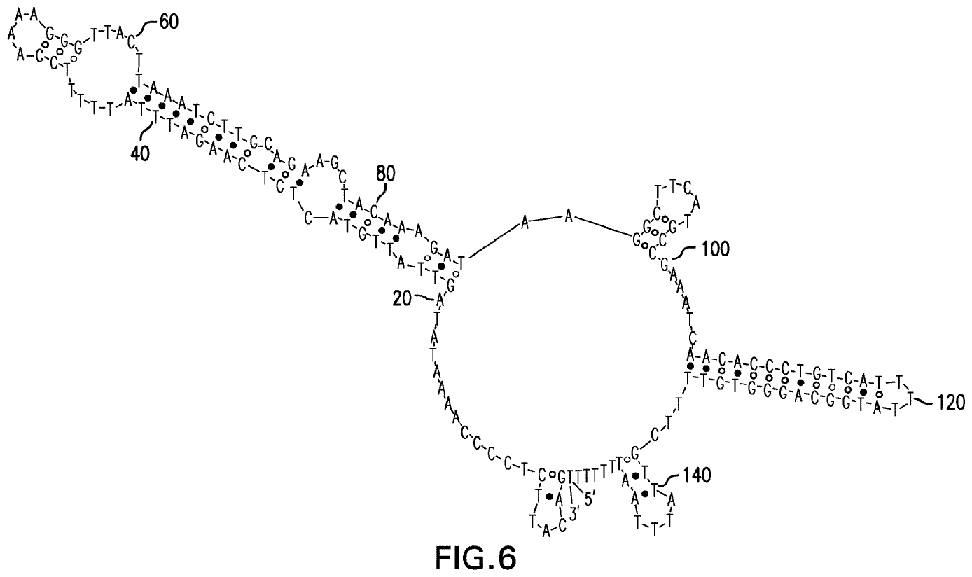

It may be desirable to accomplish CRISPR-mediated genome modification of some plants (e.g., crop plants) with CRISPR complex genes derived from Streptococcus thermophilus instead of S. pyogenes. The inventors have developed an expression cassette encoding a codon-optimized nucleotide sequence with two nuclear localization signals (NLS) (SEQ ID NO:136) of the Cas9 protein from S. thermophilus (SEQ ID NO:69). The StCas9 was designed to encode both an N-terminal and a C-terminal nuclear-localization signal (NLS) (SEQ ID NO:120) at amino acid position 2-11 and 1133-1142 (SEQ ID NO:135). Additionally, the DNA expression cassette (SEQ ID NO:136) included an intron at nucleotide position 507-695. A series of unique S. thermophilus single-guide RNAs (sgRNA) have been designed. The S. thermophilus sgRNA was designed to link the native S. thermophilus crRNA and tracrRNA with a stem loop (5′-CCAAAAGG-3′; SEQ ID NO:105), and to contain the spacer sequence complementary to the protospacer of the corn genomic target sites selected from Zm_L70e (SEQ ID NO:72), Zm_L70f (SEQ ID:73), Zm_L70g (SEQ ID NO:74), or Zm_L70h (SEQ ID NO:75). The seven nucleotides at the 3′ end of each of these genomic target sites represent the S. thermophilus-specific protospacer adjacent motif (PAM, 5′-NNAGAAW-3′; SEQ ID NO:106). FIG. 6 shows the predicted secondary structure of this S. thermophilus sgRNA (SEQ ID NO:70) with a copy of the spacer sequence (SEQ ID NO:71) complementary to the protospacer sequence of the corn Zm_L70 h genomic target site (SEQ ID NO:75) and stem-loop linker (5′-CCAAAAGG-3′; SEQ ID NO:105). Table 6 lists the corresponding SEQ ID NOs for the DNA and RNA sequences encoding S. thermophilus sgRNAs containing spacer sequences complementary to the protospacer sequence of the corn genomic target sites Zm_L70e, Zm_L70f, Zm_L70g, and Zm_L70h.

TABLE 6
DNA and RNA sequences of Streptococcus therrnophilus
sgRNA containing spacer sequences complementary
to the protospacer sequence of the corn genomic target
sites Zm_L70e, Zm_L70f, Zm_L70g, and Zm_L70h.

| SEQ ID NO. | | |
|---|---|---|
| DNA | RNA | Genomic target site |
| 107 | 111 | Zm_L70e |
| 108 | 112 | Zm_L70f |
| 109 | 113 | Zm_L70g |
| 110 | 114 | Zm_L70h |

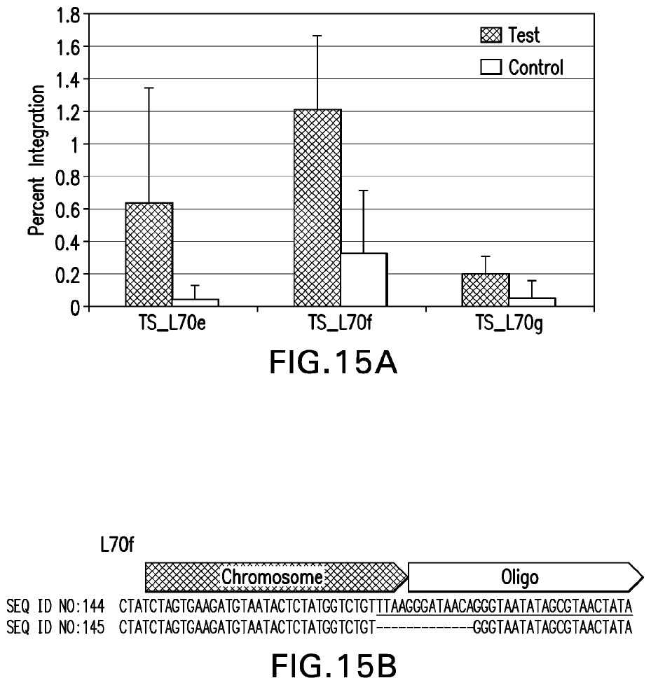

The assay for S. thermophilus Cas9 mediated genome modification was essentially as described in example 5. Specifically, 320,000 corn protoplasts were transfected with 0.8 pmol S. thermophilus Cas9 (SEQ ID NO:136) expression construct, and 1.6 pmol of one of the sgRNA expression constructs driven by the 397 bp version of the U6 promoter from corn chromosome 8 (SEQ ID NO:7) containing the spacer sequence corresponding to the genomic target sites: sgRNA construct for site L70e (SEQ ID NO:107), sgRNA construct for site L70f (SEQ ID NO:108, and sgRNA construct for site L70g (SEQ ID NO:109), and 50 pmol of a pre-annealed blunt-end double-strand DNA fragment encoded by SEQ ID NO:115 and SEQ ID NO:116. To test for transformation efficiency, 2.5 ug of a construct encoding green fluorescent protein (GFP) was included. At the time of harvesting, an aliquot of the transfected protoplasts was collected to calculate transfection frequency on the PE Operetta® Imaging System (PerkinElmer, Waltham, MA) which calculates the ratio of GFP positive cells per total cells. Omission of the StCas9 expression cassette during the corn protoplast transformation served as the negative control. Protoplasts were harvested 48 hours post transfection and analyzed for insertion of the blunt-end double-strand DNA fragment into the L70e, or L70f, or L70g genomic target site by quantitative, high-throughput PCR analysis using a BioRad QX200™ Droplet Digital™ PCR (ddPCR™) system (BioRad, Hercules, CA) and TaqMan® probes. To determine the percent targeted integration rate, one set of TaqMan primers and probes was used with the ddPCR system to detect the template copy number of a junction of the inserted blunt-end double-strand DNA fragment at the chromosomal target site. The junction specific primers and probe for corn chromosomal sites L70e, L70f, L70g, and L70 h are indicated in Table 7. To normalize the amount of DNA in the transfected protoplast aliquot, the ddPCR system was used with a second set of TaqMan primers and a probe (primers encoded by SEQ ID NO:132 and SEQ ID NO:134; probe encoded by SEQ ID NO:133) to determine the template copy number of a site unique in the corn genome and outside of the target site. The calculation for the percent targeted integration rate was the target site specific template copy number divided by the corn genome specific template copy number divided by the transformation frequency as determined by GFP-positive vs. total cell counts using the PE Operetta® Imaging System (PerkinElmer, Waltham, MA). The data points presented in the graph were determined by averaging four biological replicates. The results are presented in FIG. 15 and show that the percent integration rate for each of the sites L70e, L70f, and L70g was higher than the corresponding control.

PCR amplicons corresponding to targeted junctions from the protoplast experiments were sequenced to confirm the integration of the blunt-end double-strand DNA fragments into the selected target sites. FIG. 15B shows an alignment of the expected integration of the blunt-end, double-strand DNA fragment at the L70f target site (SEQ ID NO:144) and one example of target site integration (SEQ ID NO:145) with deletion of some of the sequence of the DNA fragment. Although these sequencing results show indels, the results confirm that the DNA fragment was integrated at the L70f target site.

TABLE 7
SEQ ID NOs for primers and probes for PCR
amplification of junction at corn chromosomal
target sites with inserted DNA fragment.

| | SEQ ID NO: | SEQ ID | SEQ ID NO: of |
|---|---|---|---|
| | of Genomic | NO: of | Inserted DNA |
| Site | specific primer | Probe | specific primer |
| L70e | 139 | 138 | 137 |
| L70f | 140 | 138 | 137 |
| L70g | 141 | 138 | 137 |
| L70h | 142 | 138 | 137 |

### Example 7

### Targeting Multiple Unique Genomic Sites by sgRNA Multiplexing

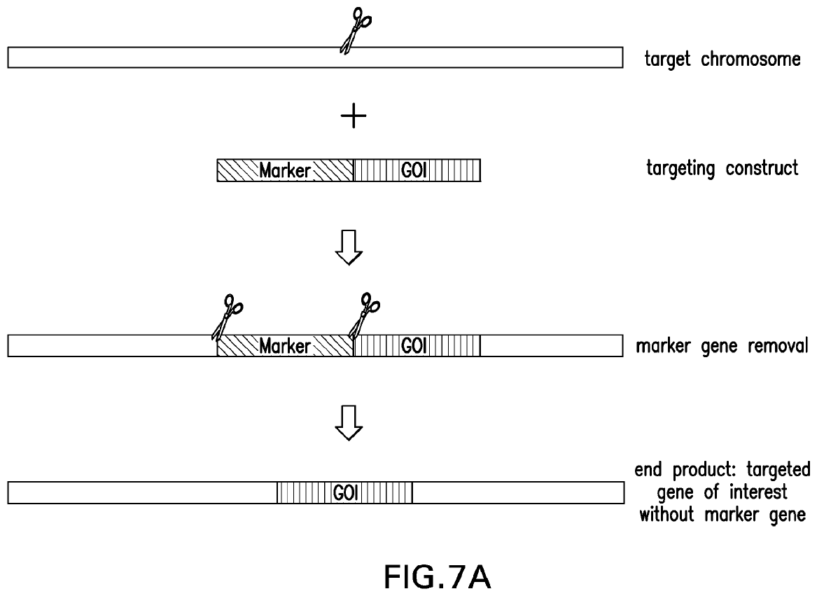

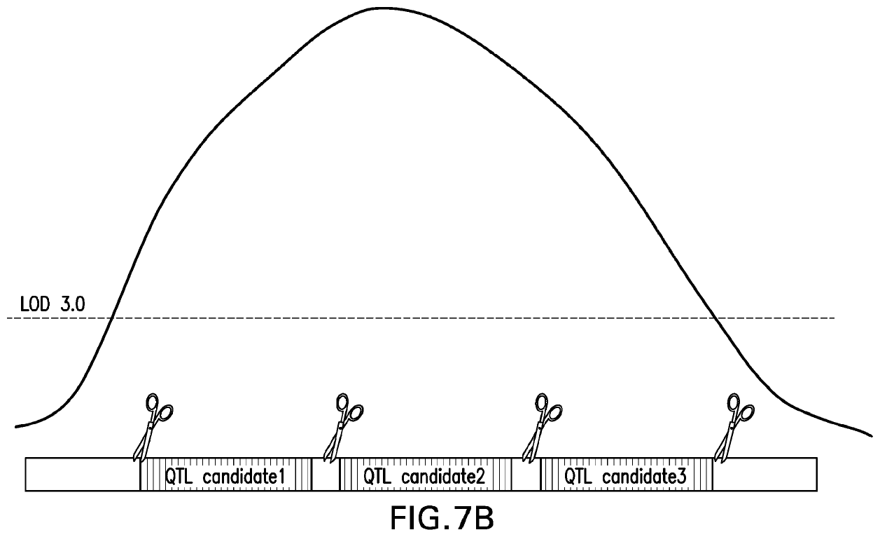

A key advantage of the CRISPR system, as compared to other genome engineering platforms, is that multiple sgRNAs directed to separate and unique genomic target sites can be delivered as individual components to effect targeting. Alternatively, multiple sgRNAs directed to separate and unique genomic target sites can be multiplexed (i.e., stacked) in a single expression vector to effect targeting. An example of an application that can require multiple targeted endonucleolytic cleavages includes marker-gene removal from a transgenic event (FIG. 7A). The CRISPR system can be used to remove the selectable marker from the transgenic insert, leaving behind the gene of interest.

Another example of an application in which such a CRISPR/Cas system can be useful is when there is a requirement for multiple targeted endonucleolytic cleavages, such as when the identification of causal genes behind a quantitative trait is hampered by lack of meiotic recombinations in the QTL regions that would separate the gene candidates from each other. This can be circumvented by transformation with several CRISPR constructs targeting the genes of interests simultaneously. These constructs would either knock out the gene candidates by frame shift mutations or remove them by deletion. Such transformations can also lead to random combinations of intact and mutant loci that would allow for identification of casual genes (FIG. 7B).

### Example 8

### Integration Rates as a Function of Blunt-End DNA Fragment Concentration and Time

The corn protoplast system essentially as described in Example 5 was used to determine the optimal concentration of blunt-end double-strand DNA fragment to be included in the assay mixture to achieve the highest percentage targeting integration rate. For these as says the expression construct encoding the S. pyogenes Cas9 was modified to include an intron from position 469-657 in the coding region (SEQ ID NO:119). Additionally, the protein sequence (SEQ ID NO:118) contained two NLS sequences (SEQ ID NO:120), one at the amino-terminal end (amino acids 2 to 11 of SEQ ID NO:118) and one at the carboxy-terminal end (amino acids 1379 to 1388 of SEQ ID NO:118).

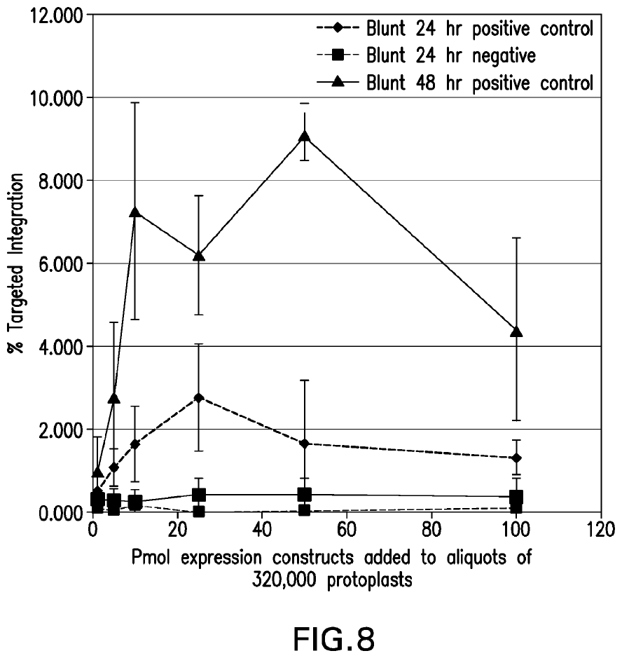

For the assay, 320,000 corn protoplasts were transfected with 0.8 pmol S. pyogenes Cas9 (SEQ ID NO:119) expression construct, and 1.6 pmol of sgRNA expression construct driven by the 397 bp version of the U6 promoter from corn chromosome 8 (SEQ ID NO:7) containing the spacer sequence corresponding to the genomic target sites: Zm7 (SEQ ID NO:23), and a pre-annealed blunt-end double-strand DNA fragment (SEQ ID NO:115 and SEQ ID NO:116) at 1, 5, 10, 25, 50, and 100 pmol. For transformation efficiency, 2.5 ug of a construct encoding green fluorescent protein (GFP) was included and the number of GFP positive protoplasts per 320,000 corn protoplasts was determined. Omission of the Cas9 expression cassette during the corn protoplast transformation served as the negative control. Protoplasts were harvested at 24 hours and 48 hours post transfection and analyzed for insertion of the blunt-end double-strand DNA fragment into the Zm7 genomic target site by quantitative, high-throughput PCR analysis using a BioRad QX200™ Droplet Digital™ PCR (ddPCR™) system (BioRad, Hercules, CA) and Tagman® probes. To determine the percent targeted integration rate, one set of Tagman primers (represented by SEQ ID NO:137 and SEQ ID NO:143) and a probe (represented by SEQ ID NO:138) was used with the ddPCR system to detect the template copy number of a junction of the inserted blunt-end double-strand DNA fragment at the chromosomal Zm7 target site. To normalize the amount of DNA in the transfected protoplast aliquot, the ddPCR system was used with a second set of Taqman primers and a probe (primers encoded by SEQ ID NO:132 and SEQ ID NO:134; probe encoded by SEQ ID NO:133) to determine the template copy number of a site unique in the corn genome and outside of the target site. The calculation for the percent targeted integration rate was the target site specific template copy number divided by the corn genome specific template copy number divided by the transformation frequency as determined by GFP-positive vs. total cell counts using the PE Operetta Imaging System (PerkinElmer, Waltham, MA). The data points presented in the graph were determined by averaging four biological replicates. The results are presented in FIG. 8 and show that the peak for percentage targeted integration rate was obtained with 50 pmol of the blunt-end, double-strand DNA fragment and incubation for 48 hours.

### Example 9

### Integration Rates as a Function of Cas9 Endonuclease Concentration

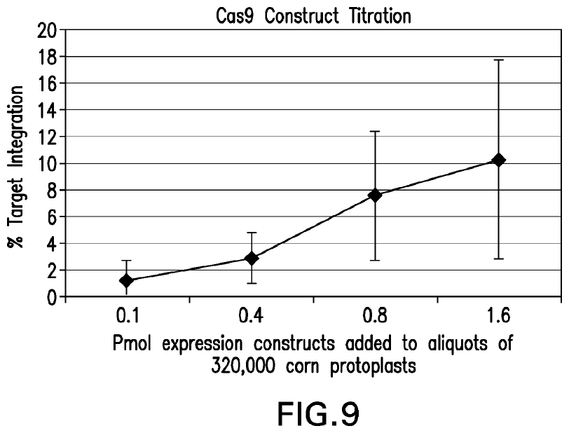

The corn protoplast system essentially as described in Example 8 was used to establish the optimal concentration of expression constructs encoding S. pyogenes Cas9 included in the protoplast transfection mixture to achieve the highest percentage targeted integration rate with the blunt-end double-strand DNA fragments. For these assays the expression construct encoding the modified S. pyogenes Cas9 was as described in Example 8. For the assay, 320,000 corn protoplasts were transfected with 0.1 pmol or 0.4 pmol or 0.8 pmol or 1.6 pmol of the S. pyogenes Cas9 (SEQ ID NO:119) expression construct, and 1.6 pmol of sgRNA expression construct driven by the 397 bp version of the U6 promoter from corn chromosome 8 (SEQ ID NO:7) containing the spacer sequence corresponding to the genomic target site Zm7 (SEQ ID NO:23), 50 pmol of pre-annealed blunt-end double-strand DNA fragment (SEQ ID NO:115 and SEQ ID NO:116), and a construct encoding GFP. The corn protoplasts were harvested 48 hours post-transfection and the percentage targeted integration was assessed as described in Example 8 using the ddPCR system and Taqman probes. The results of the analysis of the Cas9 expression construct titration are presented in FIG. 9 showing a linear increase in percentage targeted integration rate over the full-range of pmol of expression construct concentration tested.

### Example 10

### Sequence Confirmation of Insertion of Blunt-End Double-Strand DNA Fragments

PCR amplicons corresponding to targeted junctions from the protoplast experiments detailed in Example 5 and Example 8 were sequenced to confirm the integration of the blunt-end double-strand DNA fragments into the selected target site, Zm7 or L70c.

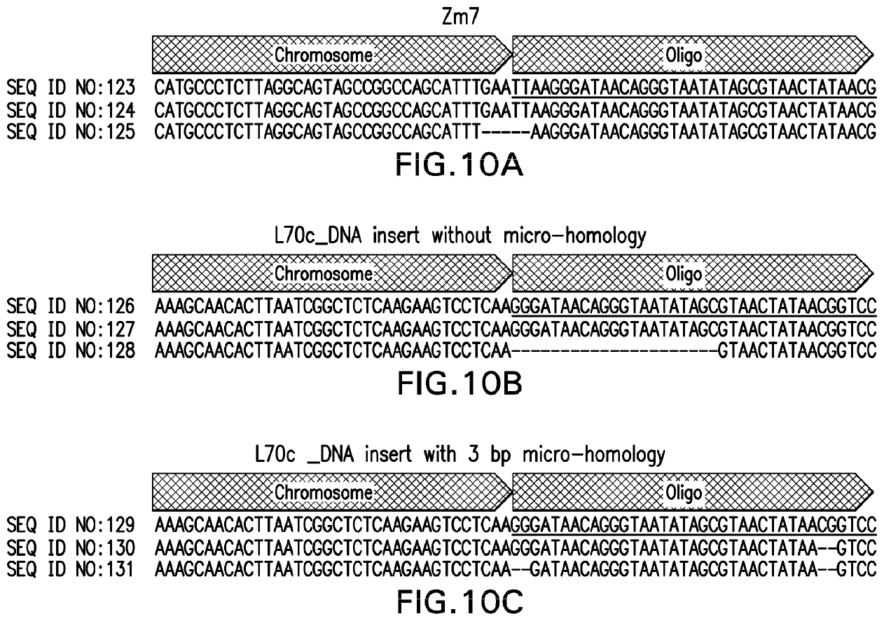

For the corn chromosome site Zm7 targeted by CRISPR/Cas9 constructs and with blunt-end double-strand DNA fragment formed by annealed oligonucleotides encoded by SEQ ID NO:115 and SEQ ID NO:116 (see Example 8), PCR amplicons were agarose-gel purified and sequenced. The expected sequence is presented as SEQ ID NO:123, as shown in FIG. 10A. The results from the sequencing show at least one event with a base-pair perfect insertion of the blunt-end double-strand DNA fragment into the target site (SEQ ID NO:124). The results also show events with short deletions in either the chromosome or the DNA insert side of the junction, as indicated with SEQ ID NO:125 (see FIG. 10A).

For the corn chromosome site L70c targeted by CRISPR/Cas9 constructs and with blunt-end double-strand DNA fragment without micro-homology sequences formed by annealed oligonucleotides encoded by SEQ ID NO:45 and SEQ ID NO:46 (see Example 5), PCR amplicons were agarose-gel purified and sequenced. The expected sequence is presented as SEQ ID NO:126, as shown in FIG. 10B. The results from the sequencing show at least one event that was detected with a base-pair perfect insertion of the blunt-end double-strand DNA fragment into the target site (SEQ ID NO:127). The results also show an example of events with short deletions in either the chromosome or the DNA insert side of the junction, as indicated with SEQ ID NO:128 (see FIG. 10B).

For the corn chromosome site L70c targeted by CRISPR/Cas9 constructs and with blunt-end double-strand DNA fragment with 3 bp micro-homology sequences at each end of the DNA fragment formed by annealed oligonucleotides encoded by SEQ ID NO:121 and SEQ ID NO:122 (see Example 5), PCR amplicons were agarose-gel purified and sequenced. The expected sequence is presented as SEQ ID NO:129, as shown in FIG. 10C. The results from the sequencing show at least one event that was detected with a base-pair perfect insertion at the junction of the blunt-end double-strand DNA fragment into the target site (SEQ ID NO:130). The results also show an example of events with short deletions in either the chromosome or the DNA insert side of the junction (SEQ ID NO:131) and/or in the DNA insert itself (SEQ ID NO:130 and SEQ ID NO:131), as indicated (see FIG. 10C).

These results indicate that blunt-end double-strand DNA fragments are incorporated into a double-strand break (DSB) at a target site created by a CRISPR/Cas9 system. The DNA fragments are incorporated by non-homologous end joining (NHEJ), an error-prone DNA repair mechanism that heals most somatic double-strand breaks in nature. Consistent with the endogenous NHEJ repair mechanism, the results show that blunt-end double-strand DNA fragments were incorporated with short deletions at the DSB created with CRISPR/Cas9 components, as illustrated by comparing SEQ ID NO:123 and SEQ ID NO:125 (FIG. 10A), and by comparing SEQ ID NO:126 and SEQ ID NO:128 (FIG. 10B), and by comparing SEQ ID NO:129 and SEQ ID NO:131 (FIG. 10C) (with this last pair there was also a 2 bp deletion internal to the inserted DNA fragment). Blunt-end double-strand DNA fragments were incorporated in a base-pair perfect manner at the DSB created with CRISPR/Cas9 components, as illustrated by comparing SEQ ID NO:123 and SEQ ID NO:124 (FIG. 10A), and by comparing SEQ ID NO:126 and SEQ ID NO:127 (FIG. 10B), and by comparing SEQ ID NO:129 and SEQ ID NO:130 (FIG. 10C) (though in this last pair there was a 2 bp deletion internal to the inserted DNA fragment).

### Example 11

### Integration Rates as a Function of TALEN Endonuclease Concentration

The corn protoplast system essentially as described in Example 8 was used to establish the optimal concentration of expression constructs encoding a pair of TALEN endonucleases needed in the transfection mixture to achieve the highest percentage targeting integration rate of blunt-end double-strand DNA fragments.

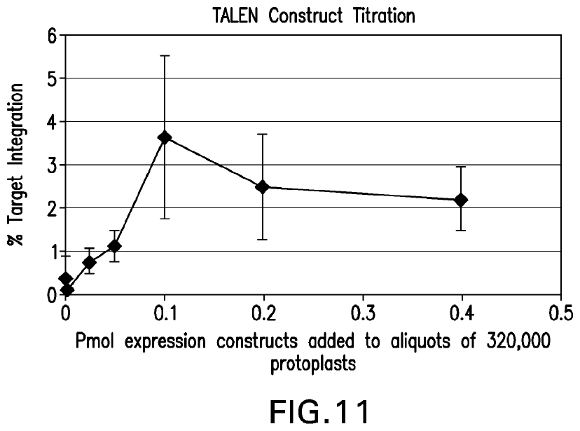

For these assays a pair of expression constructs with TALEN encoding cassettes was tested. The targeting site in the corn chromosome for the TALEN pair was L70.4. For the TALEN assay 0, 0.01, 0.02, 0.05, 0.1, 0.2 and 0.4 pmol of each of the constructs containing the TALEN encoding cassettes was used in the corn protoplast transformation. Also included was 50 pmol of pre-annealed blunt-end double-strand DNA fragment (SEQ ID NO:115 and SEQ ID NO:116) and 2.5 ug of the GFP encoding construct. The corn protoplasts were harvested 48 hours post-transfection and the percentage targeted integration was assessed by high-throughput PCR analysis essentially as described in previous examples. The results of the analysis of the TALEN expression construct titration are presented in FIG. 11 showing that the percentage targeted integration rate plateaus at about 0.1 pmol of each of the TALEN expression constructs included in the transfection reaction.

### Example 12

### Targeted Integration by Homologous Recombination—CRISPR/Cas9

Genome modification by targeted integration of a desired introduced DNA sequence will occur at sites of double strand breaks (DSB) in a chromosome. The integration of the DNA sequence is mediated by mechanisms of non-homologous end-joining (NHEJ) or homologous recombination using DNA repair mechanisms of the host cell. DSBs at specific sites in the host cell genome can be achieved using an endonuclease such as an engineered meganuclease, an engineered TALEN or a CRISPR/Cas9 system.

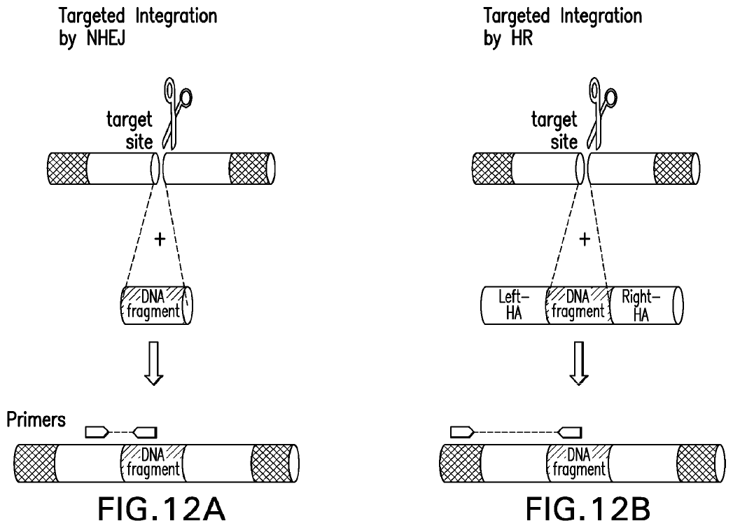

A schematic representation of a high through-put (HTP) testing method of NHEJ and HR-mediated targeted integration is presented in FIG. 12. Targeted integration of a DNA fragment by non-homologous end-joining (NHEJ) is presented in FIG. 12A and targeted integration of a DNA fragment by homologous recombination (HR) is presented in FIG. 12B. For HR, a recombinant DNA construct containing a cassette with the DNA fragment flanked with left- and right-homology arms (Left-HA and Right-HA, respectively) is introduced into the host cell. Following either NHEJ or HR targeted integration, HTP PCR analysis with primers (indicated by the short pair of arrows in FIGS. 12A and 12B) designed to detect a targeted event where one primer is internal to the inserted DNA fragment and a second primer is located in the flanking chromosomal region.

The corn protoplast system as described in the above examples was used to determine homologous recombination (HR) mediated targeted integration rates. The target site Zm7 was targeted by a CRISPR/Cas9 nuclease and the sgRNA for targeting the corn Zm7 site, as described in Example 8. In addition to the constructs encoding the CRISPR/Cas9 and sgRNA cassettes, a construct containing a cassette for homologous recombination cassette was included at either 4 ug concentration or 6 ug concentration. As described above, a construct encoding GFP was also transfected and the percentage of GFP positive cells was used in the calculation of the targeted integration rate. The controls did not contain the construct encoding the SpCas9 endonuclease.

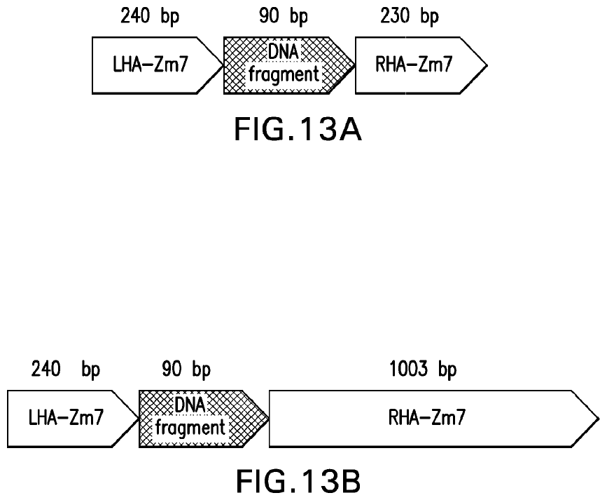

The recombinant DNA constructs containing cassettes for homologous recombination were designed to have the 90 bp sequence corresponding to the 90 bp blunt-end, double-strand DNA fragment used for NHEJ assays (encoded by sequences SEQ ID NO:115 and SEQ ID NO:116) flanked by left and right homology arms (HA). The left-HA is designed based on the sequence flanking the 5′-side of the site for the double-strand break (DSB) for targeted integration. The right-HA is designed as the sequencing flanking the 3′-side of the site for the double-strand break (DSB) for targeted integration. For the Zm7 site the left-HA was 240 bp in length, and two separate right-HA sequences were included, one of 230 bp and one of 1003 bp in length (see FIGS. 13A and 13B, respectively).

Protoplasts were transfected and harvested 48 hours later and analyzed for integration by high through-put PCR with one primer designed for the region of the DNA fragment sequence (encoded by the sequences SEQ ID NO:115 and SEQ ID NO:116) and one primer in the chromosomal region flanking the left homology arm. The size of the expected PCR amplicon with successful HR using the Zm7 targeting constructs (FIGS. 13A and 13B) was 411 bp. In conventional quantitative PCR (qPCR), amplicons longer than about 160 bp cannot be quantitatively measured, and thus, are not recommended to be used. The current experiment clearly demonstrated that significantly longer PCR amplicons can also be used in the ddPCR system, which opens up a host of new opportunities in quantitative biology.

The HR-mediated recombination rate for the corn chromosomal site Zm7 are presented in Table 8 and FIG. 15. When the left-HA and the right-HA were 240 bp and 230 bp, respectively, and the construct with the homology arm cassette was at a concentration of 4 ug or 6 ug, there was not a statistically significant difference in the percentage integration rate between the test sample and the control. When the left-HA was 240 bp and the right-HA was 1003 bp (indicated by SL in Table 8), and the construct with the homology arm cassette was at a concentration of 4 ug there was not a statistically significant difference in the percentage integration rate between the test sample and the control. In contrast, when the left-HA was 240 bp and the right-HA was 1003 bp (indicated by SL in Table 8), and the construct with the homology arm cassette was at a concentration of 6 ug there was a statistically significant (p<0.05) difference in the percentage integration rate between the test sample and the control. This result shows that targeted integration can be achieved by the mechanism of HR at sites of DSB which are targeted by CRISPR/Cas9 system in a corn genome.

TABLE 8
HR-mediated integration rates in corn protoplasts with DSB
mediated by a CRISPR/Cas9 system at the chromosomal site Zm7.

| | Mean | | Std Dev | |
|---|---|---|---|---|
| | Test | Control | Test | Control |
| Zm7 + SS + 4 ug | 0.88346 | 0.15936 | 0.83999 | 0.17658 |
| Zm7 + SS + 6 ugl | 1.20057 | 0.15936 | 0.92889 | 0.17658 |
| Zm7 + SL + 4 ug | 1.297183 | 0.98692 | 0.791837 | 0.86133 |
| Zm7 + SL + 6 ug** | 2.32094 | 0.98692 | 1.35951 | 0.86133 |

**Test was statistically higher (p < 0.05) than the corresponding control based on a student’s t-test.

### Example 13

### Targeted Integration by Homologous Recombination—TALEN

The corn protoplast system as described in the above examples was used to determine homologous recombination (HR) mediated targeted integration rates. The target site L70.4 was targeted by a pair of recombinant DNA constructs encoding a TALEN pair directed to target the corn L70.4 site, as described in Example 11. In addition to the constructs encoding the TALEN cassettes, a construct containing a cassette for homologous recombination cassette was included at either 4 ug concentration or 6 ug concentration. As described above, a construct encoding GFP was also transfected and the percentage of GFP positive cells was used in the calculation of the targeted integration rate. The controls did not contain the constructs encoding the TALENs.

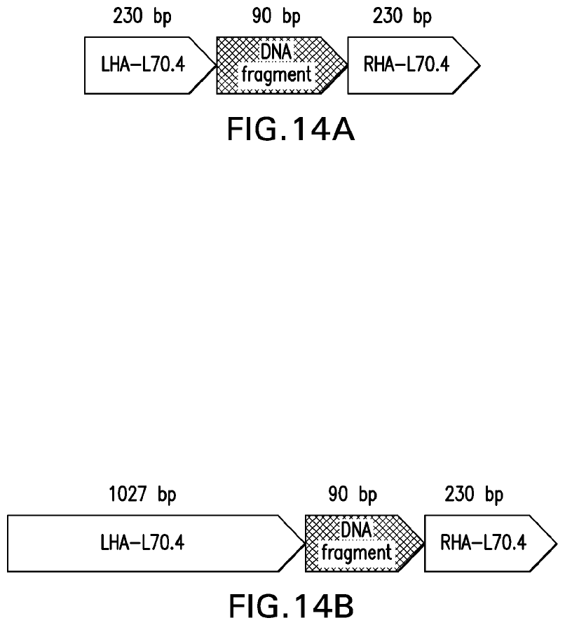

The recombinant DNA constructs containing cassettes for homologous recombination were designed to have the 90 bp sequence corresponding to the 90 bp blunt-end, double-strand DNA fragment used for NHEJ assays (encoded by sequences SEQ ID NO:115 and SEQ ID NO:116) flanked by left and right homology arms (HA). The left-HA is designed based on the sequence flanking the 5′-side of the site for the double-strand break (DSB) for targeted integration. The right-HA is designed as the sequencing flanking the 3′-side of the site for the double-strand break (DSB) for targeted integration. For the L70.4 site the right-HA was 230 bp in length, and two separate left-HA sequences were included, one of 230 bp and one of 1027 bp in length (see FIGS. 14A and 14B, respectively).

Protoplasts were transfected and harvested 48 hours later and analyzed for integration by quantitative, high through-put PCR using the ddPCR system and Taqman probes with one primer designed for the region of the DNA fragment sequence (encoded by the sequences SEQ ID NO:115and SEQ ID NO:116) and one primer in the chromosomal region flanking the left homologous arm. The size of the expected PCR amplicon with successful HR using the L70.4 targeting construct of FIG. 14A was 383 bp. The size of the expected PCR amplicon with successful HR using the L70.4 targeting construct of FIG. 14B was 1208 bp.

The HR-mediated recombination rate for the corn chromosomal site L70.4 with two separate template DNA constructs is presented in Table 9. When the left-HA and the right-HA were both 230 bp (indicated by SS in Table 9), and the construct with the homology arm cassette was at a concentration of 4 ug there was a statistically significant (p<0.05) difference in the percentage integration rate between the test sample and the control. When the left-HA and the right-HA were both 230 bp (indicated by SS in Table 9), and the construct with the homology arm cassette was at a concentration of 6 ug there was not a statistically significant difference in the percentage integration rate between the test sample and the control. When the left-HA was 1027 bp and the right-HA was 230 bp (indicated by LS in Table 9), and the construct with the homology arm cassette was at a concentration of 4 ug or 6 ug there was not a statistically significant difference in the percentage integration rate between the test sample and the control. This result shows that targeted integration can be achieved by the mechanism of HR at sites of DSB which are targeted by TALENs directed to a specific site in a corn genome.

TABLE 9
HR-mediated Integration Rates in corn protoplasts with DSB
mediated by TALENs at the chromosomal site L70.4.

| | Mean | | Std Dev | |
|---|---|---|---|---|
| | Test | Control | Test | Control |
| L70.4 + SS + 4 ug** | 1.54833 | 0.12181 | 1.48997 | 0.14504 |
| L70.4 + SS + 6 ug | 0.28395 | 0.12181 | 0.20174 | 0.14504 |
| L70.4 + LS + 4 ug | 0.163347 | 0.38048 | 0.282926 | 0.67502 |
| L70.4 + LS + 6 ug | 0.51467 | 0.38048 | 0.23052 | 0.67502 |

**Test was statistically higher (p < 0.05) than the corresponding control based on a student’s t-test.

### Example 14

### Targeting in Corn Genome with Chimeric U6 Promoters

Chimeric U6 promoters were determined to be effective at driving expression of sgRNA constructs and resulting in targeted integration of double-strand, blunt-end DNA fragments at preselected sites in corn chromosomes. These experiments were conducted using the quantitative chromosome cutting assay in corn protoplast assay as described in example 5 and example 6. The U6 promoters incorporated into the sgRNA constructs were: a) the 397 bp corn chromosome 8 U6 promoter encoded by SEQ ID NO:7, b) the 397 bp ch1:ch8 chimeric U6 promoter encoded by SEQ ID NO:18, b) the 397 bp ch8:ch1 chimeric U6 promoter encoded by SEQ ID NO:19, and c) the 397 bp ch8:ch2:ch1:ch8 chimeric U6 promoter encoded by SEQ ID NO:20. The corn chromosomal target sites were L70a, L70c, and L70d, as described in example 5. The CRISPR/Cas9 system employed an expression cassette with the S. pyogenes Cas9 modified to contain two NLS sequences and an intron and encoded by SEQ ID NO:119. The double-strand, blunt-end DNA fragment was encoded by SEQ ID NO:115 and SEQ ID NO:116.

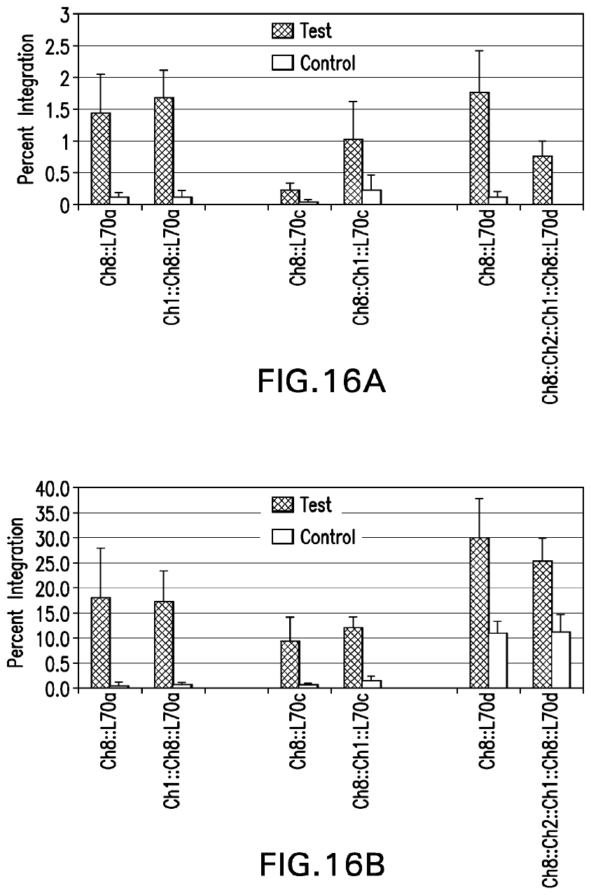

In one assay, 48 hours post transfection of the corn protoplasts with the CRISPR/Cas9 system components, the quantitative assay was done with TaqMan probes. The results (see FIG. 16A) indicate that the targeted integration rate at target site L70a with the sgRNA construct containing the ch8 U6 promoter or the sgRNA construct containing the chimeric ch1:ch8 U6 promoter resulted in about the equivalent percent target integration rate. The targeted integration rate at target site L70c, the sgRNA construct containing the chimeric ch8:ch1 U6 promoter resulted in about double the target integration rate compared to sgRNA construct containing the ch8 U6 promoter. The targeted integration rate at target site L70d, the sgRNA construct containing the ch8 U6 promoter had higher targeted integration rate compared to the sgRNA construct containing the chimeric ch8:ch2:ch1:ch8 U6 promoter.

In another assay, 48 hours post transfection of the corn protoplasts with the CRISPR/Cas9 system components, the quantitative assay was done with EvaGreen® (BioRad, Hercules, CA) intercalating dye. The results (see FIG. 16B) indicate that the targeted integration rate with the sgRNA construct containing the ch8 U6 promoter was nearly the same as the targeted integration rate at target site L70a with the sgRNA construct containing the chimeric ch1:ch8 U6 promoter, and at target site L70c with the sgRNA construct containing the chimeric ch8:ch1 U6 promoter, and at target site L70d with the sgRNA construct containing the chimeric ch8:ch2:ch1:ch8 U6 promoter. These data indicate that the targeted integration rate detected by the EvaGreen intercalating dye was about ten-fold higher compared to the targeted integration rates detected using MGB TaqMan probes. This discrepancy is mostly due to differences in the chemistries of the assays. The TaqMan assay uses just two primers and an internal probe, of which one of the primers and the probe are located on the inserted DNA fragment sequence. Unfortunately, the double-strand, blunt-end DNA fragment used in the transfection often undergo degradation by endogenous exonucleases in the protoplasts, and this results in DNA fragment integrations with truncated sites where the TaqMan probe binds. These truncated integration events are not detectable by the TaqMan assay. On the other hand, the binding site for the TaqMan primer located within the inserted DNA fragment sequence is located more internally in the inserted DNA fragment and remains intact even in most truncated inserted DNA fragments. Since the assay with the intercalating Evagreen dye does not require the internal probe, and only the TaqMan primers, this assay is not affected by oligo degradations and thus can detect many more integrations than the TaqMan assay. Otherwise, the two methods of measuring the percent targeted integration showed similar patterns at the three chromosomal target sites and the three different chimeric U6 promoters driving sgRNA expression.

These results show that targeted integration rate at corn chromosomal site L70c when the sgRNA construct contains the Ch8::Ch1 chimeric promoter was slightly, to significantly higher compared to targeted integration rate when the sgRNA construct contains the ch8 U6 promoter (FIGS. 16A and 16B). These results also show that the targeted integration rate at corn chromosomal site L70a when the sgRNA construct contains the Ch1::Ch8 chimeric promoter is about equivalent compared to targeted integration rate when the sgRNA construct contains the ch8 U6 promoter (FIGS. 16A and 16B). Finally, these results show that the targeted integration rate at corn chromosomal site L70d when the sgRNA construct contains the ch8:ch2:ch1:ch8 chimeric promoter was lower compared to the targeted integration rate when the sgRNA construct contains the ch8 U6 promoter (FIGS. 16A and 16B). In conclusion, at least two of the three chimeric promoters were as good as, or better than, the best non-chimeric promoter in corn. These will have utility in multiplex targeting experiments, where the diversity of expression elements is indispensable.

### Example 15

### Targeted Mutation in Tomato Invertase Inhibitor

The CRISPR/Cas9 system was used to knock out the apoplastic invertase inhibitor gene of tomato (INVINHI1) by introducing targeted frameshift point mutations following imperfect repair of the targeted double-strand breaks by NHEJ. In an earlier study, knock-down of this gene by RNAi showed elevated fruit sugar content and increased seed weight (Jin et al. Plant Cell 21:2072-2089, 2009). Reducing or eliminating the invertase inhibitor activity by either targeted mutagenesis or RNA interference is useful to improve yield and/or quality traits in other crop species too (Braun et al. J Exp Bot 65: 1713-1735, 2014).

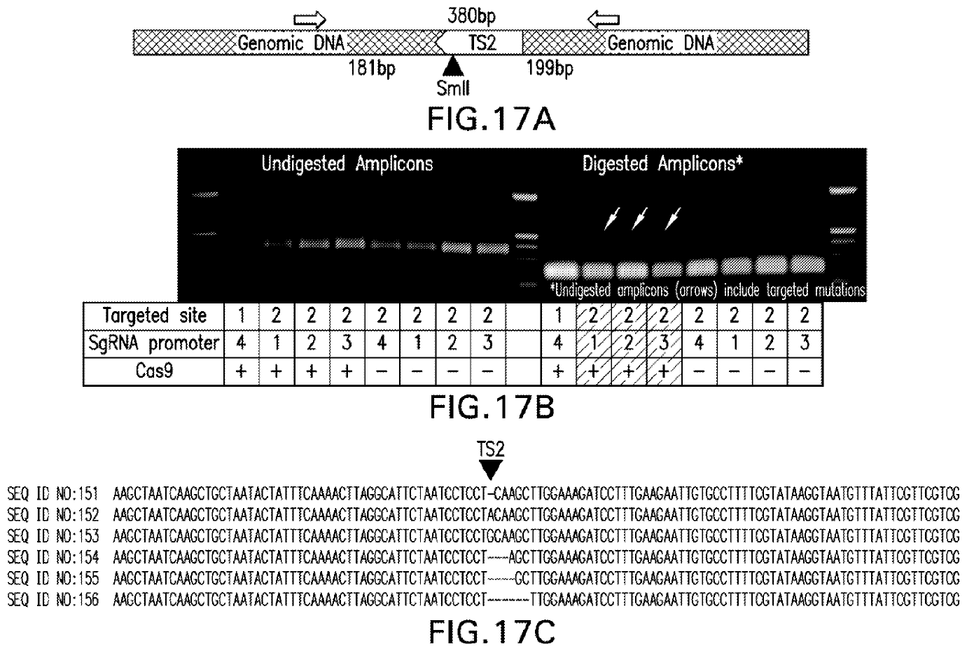

For these experiments tomato protoplasts were transfected with an expression construct containing a cassette encoding the SpCas9 with one NLS at the C-terminus (SEQ ID NO:28), and one expression construct encoding an sgRNA cassette where expression was driven by one of 4 separate tomato U6 promoters: promoter 1 encoded by SEQ ID NO:146 (which is a fragment of SEQ ID NO:10), promoter 2 encoded by SEQ ID NO:147 (which is a fragment of SEQ ID NO:11), promoter 3 encoded by SEQ ID NO:148 (which is a fragment of SEQ ID NO:9), or promoter 4 encoded by SEQ ID NO:149. The sgRNA were targeted to an invertase inhibitor site (site 1) without a SmlI site or to a site (labeled site 2) in the invertase inhibitor gene with a SmlI restriction endonuclease site. The site 2 sgRNA is encoded by SEQ ID NO:150. The CRISPR/Cas9 cleavage site within target site 2 contains a SmlI restriction endonuclease site. Upon CRISPR/Cas9 induced double-strand break at target site 2, the NHEJ repair will result in indels at this site, thus effectively removing the SmlI restriction endonuclease site. This mutation of the SmlI site was leveraged during the screening for targeted events by amplifying a 380 bp amplicon (SEQ ID NO:159) and subjecting the PCR amplicon to digestion with SmlI. If the SmlI site was not mutated, then the amplicon would be digested into two fragments of 181 bp and 199 bp. If the SmlI site was mutated, then the PCR amplicon would not be digested. This PCR scheme is illustrated in FIG. 17A.

Tomato protoplasts were transfected with the CRISPR/Cas9 system targeting the tomato invertase inhibitor and harvested 48 hours later and genomic DNA extracted. Negative control for the CRISPR/Cas9 system was omission of the expression construct encoding the Cas9 endonuclease. A negative control for the target site was use of a sgRNA to target site 1, and it is not expected that the SmlI site will be mutated with this sgRNA. PCR amplification was done with primers SEQ ID NO:157 and SEQ ID NO:158 and the resulting PCR amplicons were either undigested or digested with SmlI. The reactions were run on agarose gels and the results are shown in FIG. 17B. The negative controls of sgRNA to target site 1 and the omission of Cas9 endonuclease resulted only in PCR amplicons with the SmlI site intact. When the sgRNA was for target site 2, the SmlI site was mutated when the sgRNA cassette contained tomato U6 promoter 1, or tomato U6 promoter 2, or tomato U6 promoter 3, as evidenced by the full-length PCR amplicons (see FIG. 17B, arrows showing amplicons without a SmlI site). The sgRNA construct targeting site 2 and with U6 promoter 4 apparently did not show targeting.

To confirm that the PCR amplicons without a SmlI site were indeed due to CRISPR/Cas9 induced NHEJ mutation, these apparent mutated amplicons were gel-purified and pooled, and then they were sequenced. The multiple sequence alignment in FIG. 17C shows that these PCR amplicons without a SmlI site were from the target site 2 of the tomato invertase inhibitor and contained indels, consistent with CRISPR/Cas9 induced mutation. Specifically, in the multiple sequence alignment, SEQ ID NO:151 represents a region of the PCR amplicon (SEQ ID NO:159) without a mutation. SEQ ID NOs:152 and 153 illustrate indels where there was a 1 bp insertion at the cleavage site. SEQ ID NO:154 illustrates an indel with a 3 bp deletion at the cleavage site. SEQ ID NO:155 illustrates an indel with a 4 bp deletion at the cleavage site. SEQ ID NO:156 illustrates an indel with a 6 bp deletion at the cleavage site. In conclusion, these results indicate that the CRISPR/Cas9 system using tomato U6 promoter 1 (SEQ ID NO:146), or tomato U6 promoter 2 (SEQ ID NO:147), or tomato U6 promoter 3 (SEQ ID NO:148) to drive sgRNA induces mutation at the tomato invertase inhibitor gene target site 2.

### Example 16

### Promoters to Drive sgRNA Expression

To identify and select additional promoters which would be useful to drive expression of sgRNAs from expression cassettes introduced into dicots and monocots, RNA polymerase II (Pol II) and RNA polymerase III (Pol III) promoters (SEQ ID NOs:160-201 and SEQ ID NOs:247 283) were identified by comparing the sequence encoding U6, U3, U5, U2 and 7SL small nuclear RNA (snRNA) against soy and corn genomes using BLAST (see Table 10). From regions of this bioinformatic alignment, 200 or more nucleotides immediately upstream of the 5′ end of the coding region of the respective snRNA was used for testing as putative promoters for driving expression of sgRNA from expression cassettes introduced into plant cells.

TABLE 10
SEQ ID NO of putative promoter sequence upstream of
the snRNA genes and the source (tomato or soy or corn).

| Promoter | | | Promoter + GUS + | |
|---|---|---|---|---|
| SEQ ID | | Promoter | Terminator | |
| NO: | snRNA | Source | SEQ ID NO | Terminator |
| 148 | Promoter 3 | tomato | 202 | poly(T)7 |
| 160 | SoyU6a | soy | 203 | poly(T)7 |
| 161 | SoyU6c | soy | 204 | poly(T)7 |
| 162 | SoyU6d | soy | 205 | poly(T)7 |
| 163 | SoyU6e | soy | 206 | poly(T)7 |
| 164 | SoyU6f | soy | 207 | poly(T)7 |
| 165 | SoyU6g | soy | 208 | poly(T)7 |
| 166 | SoyU6i | soy | 209 | poly(T)7 |
| 167 | U3a | soy | 210 | poly(T)7 |
| 168 | U3b | soy | 211 | poly(T)7 |
| 169 | U3c | soy | 212 | poly(T)7 |
| 170 | U3d | soy | 213 | poly(T)7 |
| 171 | U3e | soy | 214 | poly(T)7 |
| 172 | 7SL_CR13 | soy | 215 | poly(T)7 |
| 173 | 7SL_CR14 | soy | 216 | poly(T)7 |
| 174 | 7SL_CR10 | soy | 217 | poly(T)7 |
| 175 | 7SLCR01 | corn | 218 | poly(T)7 |
| 176 | 7SLCR07 | corn | 219 | poly(T)7 |
| 177 | 7SLCR09 | corn | 220 | poly(T)7 |
| 178 | U3CR02 | corn | 221 | poly(T)7 |
| 179 | U3CR10 | corn | 222 | poly(T)7 |
| 180 | U3CR08 | corn | 223 | poly(T)7 |
| 181 | U3CR08b | corn | 224 | poly(T)7 |
| 182 | U3CR05 | corn | 225 | poly(T)7 |
| 183 | U2snRNA_P | corn | 226 | SEQ ID |
| | | | | NO 237 |
| 184 | U2snRNA_I | corn | 227 | SEQ ID |
| | | | | NO 237 |
| 185 | U2snRNA_B | corn | 228 | SEQ ID |
| | | | | NO 237 |
| 186 | U2snRNA_G | corn | 229 | SEQ ID |
| | | | | NO 237 |
| 187 | U2snRNA_A | corn | 230 | SEQ ID |
| | | | | NO 237 |
| 188 | U5snRNA_A | corn | 231 | SEQ ID |
| | | | | NO 237 |
| 189 | U5snRNA_C | corn | 232 | SEQ ID |
| | | | | NO 237 |
| 190 | U5snRNA_D | corn | 233 | SEQ ID |
| | | | | NO 237 |
| 191 | U5snRNA_E | corn | 234 | SEQ ID |
| | | | | NO 237 |
| 192 | U2snRNA_C | corn | — | — |
| 193 | U2snRNA_D | corn | — | — |
| 194 | U2snRNA_E | corn | — | — |
| 195 | U2snRNA_F | corn | — | — |
| 196 | U2snRNA_H | corn | — | — |
| 197 | U2snRNA_K | corn | — | — |
| 198 | U2snRNA_L | corn | — | — |
| 199 | U2snRNA_M | corn | — | — |
| 200 | U6Chr08 | corn | 235 | poly(T)7 |
| 201 | U6Chr01 | corn | 236 | poly(T)7 |
| 247 | U2CR01a | Soy | — | — |
| 248 | U2CR01b | Soy | — | — |
| 249 | U2CR02 | Soy | — | — |
| 250 | U2CR03 | Soy | — | — |
| 251 | U2CR04 | Soy | — | — |
| 252 | U2CR05a | Soy | — | — |
| 253 | U2CR05b | Soy | — | — |
| 254 | U2CR06a | Soy | — | — |
| 255 | U2CR06b | Soy | — | — |
| 256 | U2CR06v | Soy | — | — |
| 257 | U2CR07 | Soy | — | — |
| 258 | U2CR08a | Soy | — | — |
| 259 | U2CR08b | Soy | — | — |
| 260 | U2CR08c | Soy | — | — |
| 261 | U2CR10a | Soy | — | — |
| 262 | U2CR10b | Soy | — | — |
| 263 | U2CR10c | Soy | — | — |
| 264 | U2CR13 | Soy | — | — |
| 265 | U2CR14 | Soy | — | — |
| 266 | U2CR15 | Soy | — | — |
| 267 | U2CR17a | Soy | — | — |
| 268 | U2CR17b | Soy | — | — |
| 269 | U2CR17c | Soy | — | — |
| 270 | U2CR17d | Soy | — | — |
| 271 | U2CR17e | Soy | — | — |
| 272 | U2CR17f | Soy | — | — |
| 273 | U2CR19a | Soy | — | — |
| 274 | U2CR19b | Soy | — | — |
| 275 | U2CR20 | Soy | — | — |
| 276 | U5CR07 | Soy | — | — |
| 277 | U5CR10 | Soy | — | — |
| 278 | U5CR10 | Soy | — | — |
| 279 | U5CR15 | Soy | — | — |
| 280 | U5CR19 | Soy | — | — |
| 281 | U5CR20a | Soy | — | — |
| 282 | U5CR20b | Soy | — | — |
| 283 | SoyU6b | Soy | — | — |

### Example 17

### Normalized RNA Transcript Level Assay

To assess the efficacy of the promoters listed in Table 10 to drive expression of sgRNAs, a series of constructs were generated which contained a cassette encoding one of the putative promoters (SEQ ID NO: 154, and SEQ ID NOs:160-201) operably linked to a 221 bp fragment of a beta-glucuronidase (GUS) open reading frame and either a poly(T)7 terminator for Pol III promoters (7SL, U6, and U3) or the sequence 5′-ACAATTCAAAACAAGTTTTAT-3′ (SEQ ID NO:237) for the pol II U2 and U5 promoters (Table 10). The recombinant constructs (0.5 μmol) containing the promoter-GUS fragment fusions were transfected into soy cotyledon protoplasts (SEQ ID NO:202-217 or corn leaf protoplasts (SEQ ID NO: 218-236) along with 300 ng of a plasmid serving as a transformation control encoding Renilla Luciferase (RLUC) expressed using the CaMV promoter. The transfected protoplasts were harvested 18 hours after transfection and the RNA levels were measured via TaqMan assays using a probe and primers complementary to the GUS fragment. Internal controls used to normalized the TaqMan assay included (1) an 18S primer pair/probe set to control for RNA concentration and (2) RLUC luminescence as a transformation control.

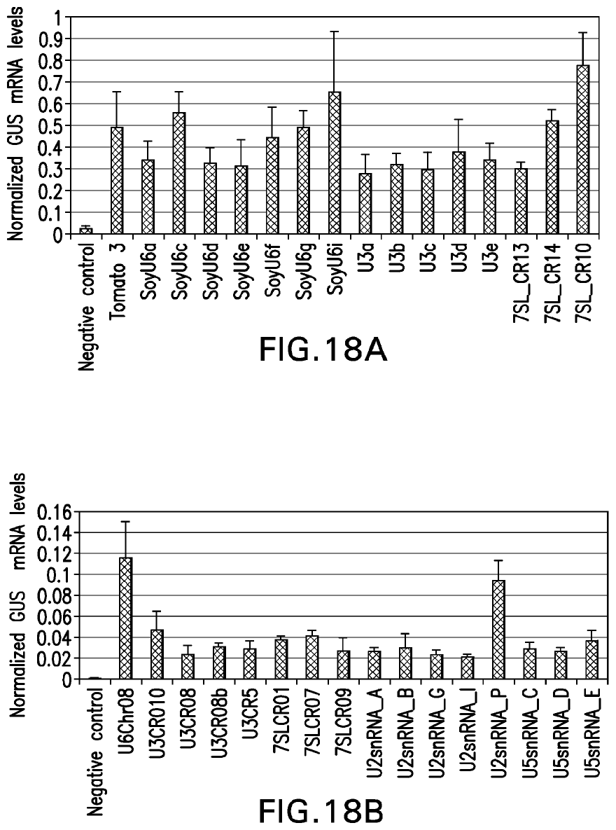

In soy cotyledon protoplasts, all promoters tested resulted in significantly higher normalized levels of GUS mRNA than the control (no GUS construct) (One-way ANOVA student t-test p value<0.05) (FIG. 18A). The lowest level of normalized GUS mRNA was with construct (SEQ ID NO:210) containing the U3a promoter (SEQ ID NO:167). The highest level of normalized GUS mRNA was with construct (SEQ ID NO:210) containing the 7SL_CR10 promoter (SEQ ID NO:174). The level of normalized GUS mRNA with all promoters tested with this assay ranged from 11-31 times higher expression levels that the no DNA negative control. No one class of promoters (U6, U3, or 7SL) performed better than the other, although the U3 promoters were generally in the lower range of expression observed in the experiment. U3 promoters have been successfully used by Liang et al. (J. Genetics and Genomics 41:63-68, 2014) to drive sgRNAs in corn. Thus, although these data indicate that the U3 promoters may be lower than U6 or 7SL, they are still viable candidates to drive sgRNA expression in soy. These data suggest that any of the U6, U3, or 7SL promoters identified here would be good candidates for making recombinant expression constructs to drive expression of sgRNA in plant cells.

In corn leaf protoplast, all promoters tested resulted in significantly higher normalized levels of GUS mRNA compared to the control (One-way ANOVA student t-test p value<0.05) with values ranging from 26 fold to 141 fold higher expression than the negative control (FIG. 18B). The U6Chr08 promoter construct (SEQ ID NO:235) resulted in the highest normalized levels of GUS mRNA expression, and U2snRNA_I promoter construct (SEQ ID NO:227) resulted in the lowest, with approximately a 5.5-fold difference in normalized levels of GUS mRNA expression between them. The U2snRNA_P promoter construct (SEQ ID NO:226) also stood out as having high normalized levels of GUS mRNA expression. All the remaining promoters were within the same relative range having less than 2 fold difference between them (FIG. 18B). These data suggest that any of the U6, U3, 7S1, U2, or U5, promoters identified here would be good candidates for making recombinant expression constructs to drive expression of sgRNA in plant cells.

### Example 18

### GUS Expression Assay for sgRNA Expression

To determine how the difference in sgRNA expression levels impact Cas9 activity, an assay was used that relied on activating transcription from a minimal promoter upstream of the GUS open reading frame in a reporter construct transfected into corn leaf protoplasts. For this assay, a Cas9 nuclease from S. thermophilus was mutated at amino acid positions D9A and H599A of the native protein sequence, effectively creating a Cas9 without endonuclease cleavage activity (also referred to as a ‘dead Cas9’). Additionally, this dead Cas9 was modified to encode one NLS domain (SEQ ID NO:120) at amino acid positions 2-11 of SEQ ID NO:239 and an activation domain from a TALE protein from amino acid positions 1135-1471 of SEQ ID NO:239. The polynucleotide sequence of the dead Cas9, represented by SEQ ID NO:238, included an intron at positions 507-695. A reporter construct was constructed where the uidA (GUS) reporter gene was driven by a minimal CaMV promoter with three adjacent sgRNA binding sites (SEQ ID NO:240) at nucleotide positions 80-98, 117-135, and 154-172 of the sequence SEQ ID NO:246. Also constructed were a set of sgRNA (based on the sgRNA of Cong et al. 2013 Science 339:819) expression constructs that consisted of the one of the promoters from each class of snRNA genes, namely U6, 7SL, U2, U5, and U3 (Table 11) and which would target the dead Cas9-TALE-AD to one or more of the sgRNA binding sites of the GUS reporter construct. The U6 and 7SL promoters normally initiate transcription on a G, and the U2, U5 and U3 promoters normally initiate transcription on an A. To ensure proper transcription initiation of the sgRNA, for constructs with either a U6 or 7SL promoter, a G was inserted between the promoter and spacer sequence. For constructs with a U2, U5 or U3 promoter, an A was inserted between the promoter and spacer sequence. When the dead Cas9-TALE-AD and sgRNA complex binds the GUS reporter construct, the TALE activation domain functions as a transcription factor activating the minimal CaMV promoter resulting in higher expression of the GUS transcript, and ultimately higher levels of GUS protein expression.

TABLE 11
SEQ ID NO corresponding to sgRNA expression constructs.

| Promotor + sgRNA | | Promotor |
|---|---|---|
| SEQ ID NO: | Promotor | SEQ ID NO: |
| 241 | U6Chr08 | 200 |
| 242 | 7SLCR07 | 176 |
| 243 | U2snRNA_I | 184 |
| 244 | U5snRNA_E | 191 |
| 245 | U3CR08b | 181 |

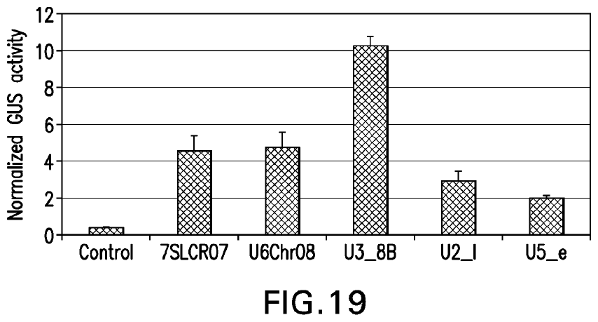

For the assay, corn leaf protoplasts were transfected with 0.8 pmol of dead Cas9-TALE-AD expression cassette, 0.5 pmol of the GUS expression cassette, 1.6 pmol of one of the sgRNA expression cassettes, 650 ng of Luciferase expression cassette, and 300 ng of Renilla Luciferase (RLUC) expression cassette. The transfected protoplasts were harvested 18 hours later and GUS activity was measured using the 4-methylumbelliferyl-beta-D-glucuronide (MUG, Sigma, St. Louis, MO) fluorimetric assay, and luciferase and RLUC activity was measured and used as control to normalize relative to transfection controls. The activity of GUS is a readout of the how often the dead Cas9-TALE-AD binds to the reporter plasmid. Each class of snRNA promoter driving sgRNA gave higher normalized GUS activity compared to the control (FIG. 19). The U3CR08b (U3_8B in FIG. 19) promoter resulted in the highest normalized GUS activity of about 10× over control. The two promoters 7SLCR07 and U6Chr08 both gave about the same normalized GUS activity of about 4× over control. The two promoters U2snRNA_I (Us_I in FIG. 19) and U5snRNA_E (U5_e in FIG. 19) were each at or slightly above 2× over control for normalized GUS activity. These results indicate that the 7SL, U6, U3, U2, and U5 snRNA promoters may be good to excellent candidates for use in sgRNA expression constructs for CRISPR/Cas9 system useful in genome modification.

The differences in normalized GUS expression observed using the dead Cas9-TALE-AD assay do not mirror the normalized GUS mRNA levels shown in the corn leaf protoplast assay detailed in Example 17.

1. A recombinant DNA construct comprising a U6 promoter operably linked to a sequence encoding a single-guide RNA (sgRNA) or a sequence specifying a non-coding RNA, wherein the sequence of said U6 promoter comprises SEQ ID NO:3, or a fragment thereof, wherein the fragment is at least 140 bp in length, and wherein the U6 promoter or fragment thereof and the sgRNA or non-coding RNA come from different sources.

2.-8. (canceled)

9. The recombinant DNA construct of claim 1, further comprising a transcription termination sequence.

10. The recombinant DNA construct of claim 1, further comprising a sequence encoding a promoter operably linked to a sequence encoding a clustered, regularly interspaced, short palindromic repeats (CRISPR)-associated Cas endonuclease gene product.

11. The recombinant DNA construct of claim 10, wherein the Cas endonuclease gene product is further operably linked to a nuclear localization sequence (NLS).

12. The recombinant DNA construct of claim 10, wherein the sequence encoding said Cas endonuclease is selected from the group consisting of SEQ ID NO:27, SEQ ID NO:68, SEQ ID NO:97, SEQ ID NO:119, and SEQ ID NO:136.

13. (canceled)

14. The recombinant DNA construct of claim 1, wherein the non-coding RNA is selected from the group consisting of: a microRNA (miRNA), a miRNA precursor, a small interfering RNA (siRNA), a small RNA (22-26 nt in length) and precursor encoding same, a heterochromatic siRNA (hc-siRNA), a Piwi-interacting RNA (piRNA), a hairpin double strand RNA (hairpin dsRNA), a trans-acting siRNA (ta-siRNA), and a naturally occurring antisense siRNA (nat-siRNA).

15.-19. (canceled)

20. A cell comprising the recombinant DNA construct of claim 1.

21. The cell of claim 20, wherein the cell is a plant cell.

22. A method of introducing a double-strand break in the genome of a cell, comprising introducing in said cell:
a) at least one recombinant DNA construct of claim 1; and
b) a second recombinant DNA construct comprising a sequence encoding a promoter operably linked to a sequence encoding a clustered, regularly interspaced, short palindromic repeats (CRISPR)-associated Cas endonuclease gene product operably linked to a nuclear localization sequence (NLS).

23.-24. (canceled)

25. The method of claim 22, wherein the sequence encoding said Cas endonuclease is selected from the group consisting of SEQ ID NO:27, SEQ ID NO:68, SEQ ID NO:97, SEQ ID NO:119, and SEQ ID NO:136.

26. A method of introducing a double-strand break in the genome of a cell, comprising introducing to said cell at least one recombinant DNA construct of claim 10.

27.-28. (canceled)

29. The method of claim 26, wherein the sequence encoding the Cas endonuclease is selected from the group consisting of SEQ ID NO:27, SEQ ID NO:68, SEQ ID NO:97, SEQ ID NO:119, and SEQ ID NO:136.

30.-59. (canceled)

60. A method of genome modification comprising:
a) introducing a double-strand break in the genome of a plant cell by the method according to claim 22; and
b) introducing into said plant cell a recombinant blunt-end double-strand DNA fragment,
wherein said recombinant blunt-end double-strand DNA fragment is incorporated into said double strand break by endogenous DNA repair.

61. A method of genome modification comprising:
a) introducing a double-strand break in the genome of a plant cell by the method according to claim 26; and
b) introducing into said plant cell a recombinant blunt-end double-strand DNA fragment,
wherein said recombinant blunt-end double-strand DNA fragment is incorporated into said double strand break by endogenous DNA repair.

62.-97. (canceled)
